SEQUENCE LISTING (80 sequences)

SEQ ID NO: 1 - DNA, 17601 bp, Unknown
  Fish coronavirus ORF-1 nucleotide sequence
  misc_feature (2490)..(2490): n is any base
  misc_feature (2491)..(2491): n is any base
  misc_feature (2492)..(2492): n is any base
  misc_feature (2493)..(2493): n is any base
  misc_feature (2494)..(2494): n is any base
  misc_feature (2495)..(2495): n is any base
  misc_feature (2496)..(2496): n is any base
  misc_feature (2497)..(2497): n is any base
  misc_feature (2498)..(2498): n is any base
  misc_feature (2499)..(2499): n is any base
  misc_feature (2500)..(2500): n is any base
  misc_feature (2501)..(2501): n is any base
  misc_feature (2502)..(2502): n is any base
  misc_feature (2503)..(2503): n is any base
  misc_feature (2523)..(2523): n is any base
  misc_feature (2524)..(2524): n is any base
  misc_feature (9771)..(9771): n is any base
atgaaaaaaattgaagatgctttaggaacacttaaacctgcctttaaggcacacatagactctctacctcctttcatgggaaaactagccacaatgctagctgagacaagacgaggaaaaactccccctcttttgatctatgtcatatcaacaattttggaaacgaatattacagttcattatgtcagccacacaataacttcataccattcttcaaacgcacacactcacactcatgaattcgactcagaagattacactcctgacaatcagattttaactaaagtcaaccgacatggtaatgaccttaatcattcatatatccatggcgccaccaatatgtacaaccctgtctaccagagacacccaccaaacatgtgctacatgcttacgggactgtacatgttgtcaggcttacaggaattgtatgccatggctgaagacaacttgacaacatgccaaatcaaccttctcagatgtctgtttgatttgaatcaagatgaatttgatgtagattacacatttgtaatatacactcctagcaaatctcaggagtgtgccttcaaatatcttcaggagatagtccaccactgtgaactcaccatttttagacatacaacaacaagtgtcttcagttgcaacaaatgcaaccatgtggaaactgtcatttctagttgttctttgaatcttgtatatataactgattctattgaaaaagcatttcaacccactgtagaagctaataccgactacatgtgtgaaaactgcggtctacgcgaccataaacttaaaacaacagtaacaaatccagacctccgtttggcacaactaaactatccaacggattcaaaatacactatctttcttgatgaacaagctccttttgtcttccattccattgcaaaacacgttggaactgctaattccggacattggagtgcattgaatgtaaattcagatatgttgtcagactctaacgagcgacaacattattatacaacacctagtattgttttgcttgcatttcttcctgaggaagagctacagaacattagaaattcatcacctcttcaggaccatcaaccagatgatgttgaagacgttgaatctcccctaccaggttctatattttatactacagatgacattttttctactaagagtctatccatagctcactgtgtagcccgcgactttcacatgtccggcggcatagcaaaaattttctcagataaattcggctctaaaactttcttgaaatcacaaaaccccgttataggcggtttttccattttacttagagagtgtcgtgacatgtactacctcgtcacaaaagagaaaacttcagataagccgacataccaagaccttaaaaactccttgggttctatgacagagaatttggttcgcaagaatcataatactctttcaattccatatataggctgtgggattgatggtttacaatgggcaaccgttgaaaaacaagtcaaagaaattgtctgcgctcgaggtattgatgtaacagtccaccacctcgaaaatgaagttaaacacactccagaacaacaaacagcttctgacaattcagtcaaattagttcaaaaactctttacagaaacacctcaagctatacctatagttgttccttcagatgactcagacagcgacatagatgagtcagccgatgtctttttaccaggacctgaatctgaatctgattcaaaatcagaatctgggtcagattacgactttaaatcggcttctgaaccagaagatgaattggagcccactccaatctctgaacttgaactaacaccagcgtcaagtttaactgtagagtccgatgacaatccagacacaagccaggaaacattaccagaatcaaactctgaggaaaccaaacctgagcaaacacctgacacaacatcaaaggtgtcctccgattcgaagcttgatccacaatcagaattagaagaagaactggccaacaaaccagaatcggcttctgaaccacaatctgagactgaatcgagttctgaatctgaagaggagcttgagccacaatcagaatcagaggaagaaccggccaacaaaccggaatctccttctgaatcacaatctgaaaatggatcgagttctgaacccgaagaggagtctgaaaaaccatcggagtctgctgaaacagcaacagaggatagcccggaaacaacaccggaaacaaccttagagttaaccacacaactcaaacctgcttcagaatctgacgacaaaccggacacaccagcaccatcaccttcaccaattcaaccagagaaaaacttggacaccacccctgaacaaacttcacaaccaaccacacaactggaattgacgttagaaacacaggaacaaccagacactacaccagaagtgccatctgtctcagaagataaaccagacacacttgaagaatcttctgaatcaacaccagaactctcagaattggannnnnnnnnnnnnnccaccagtaccaagacctanngaaccagacctccaacacctagaccaagatctgcacgtggagctaggactagatcctgtgctggcacacctataccagttatttttgatattatagataatacaagccagcctcaagttccccttgacttcccagaagcactgcaagaactgaacaaacctagtgaagtaatcccagcggctagtgaaaaacctgtggaaaaacaaataatccatagttttgtaagtgtcgaaacaccttgcaaacccaaagccactaaagttacgaattatgtagctgcacaatctaatgcaattctaaattgtattaaggctttcattcctagcaacccgctttcactattcaatagaaaaccagcttttagaaaaataatattcactgaagacacttcagaaccagatagcgatgatgatgattgtgaatacactccaccaacatcaccattccctgaacttctggcattggtagatgaagacattgaagtagaacaaactcaatctgtaattccaaaaacagactctgcttcaattgtggaggatcttaaaaaacaagaatcctctactttgtcattggacaccaacacatcgaaacctacaagctctccgcgaagacagcctagggaagtagaaagtgttgatgaatccagtgatgactcatctaaaccaaaaacaatttcaacattagacaaacctgctatgaatagtgacacgaaacctacagactcttcgcgaaaagagcctctggaagtaccagttactacatctttaagcacccctgctaaaaaccaagataaaaaatcttcaaaatctgcaaaagtaataaaagactattctttgacccctaacacagtcaaacagcaagtctattcactctacggtgaatcagtagatgcagttaaataccttgttcaaacatacccagacagggctaaacaaacagctggtattgcttatttccttataactacttatttaatatggaccatcggtctcataggagtaccaatggcctttaaaataccaatgtttttatgtcttttataccaagttaatggattaaatatagcaccatttgttactaaccaaaagttacaatatgttgcatttccactttggtataagctctatgaagtaatatcagtccgttttgtggcgaatatagcacaatttattgttaaaacaccacctatagatgttttaaacaagctaattcgttctaataaagacaagccagtcaaattaacaccaaataaacatactttgatgttaattcatgacttagctttagagtctgtcgacggaaaagaaaaccgctattataatactgatgttacaactttcacaaaaaggcatagcacttctaatatttcatatgttctaaagtctactttaatcaaatatgtcatggaccattgttatgtaaatattgcagtttttactttagttagatacttaactttattagtgtttattcaacatttctctaatccttatgttcttgaagcaaatagccaatcacataccgttttacagtatctgttttcacacttgagaccatttggaaggcctttgtgcccaaccctcaatgactacatgacgacagcaacaccacgcgatgcacatgtacaagcaggttctcacttcagtgaattttgtgttcctattcattatacaacaccaatcattaaaagcacaatggcagaaccatcactttttctactttttaacccagtcttatggcctttggttatggttgtatatttttatcctccaatgatgtttatagcaaatgcagtcgcttattcatgccttcctttagtggtcttgttacaatggctttatgccatgtggttttcttgtacatgctatggcaccaaaagatgtgccaagcatttgcataaaaatgaagtggttaaaccaatggaatccacttcaactaagaaccgcatgacatttactccatcaacgaccttttgtagtaaacataacttcttctgtccagatgcaccacatataatgactcttgcaatggctaggcaacttacaaactactacaatttgacagatacagtaatacctgacatccaggaatactcccacgagaaccctactgtacaatttattcactttgatccacttaaacacggtgccgacacaattttggaaccaattacaagcgctagcgccagttcaattgttgcatggtactctctcctctttaatcaaaagtttgtcctttcacattatagctacagaaccccagtagccgtagttgacaaaccagaggaaacagatggtgatgatacaaaatcattagcatctgacacttctgataactttgagtctattagaaagaccaaccataagaatcagagcaaacaacagtttaggccaaacggtcaccaaagaccaagtaagactttcaaacgccattcaagaataatgacatctgaacagaagaacagcttaattgaaacttttaagggtttaacaaatggcacagcagccatcccacagcctttaatcatttttatttgggttatccttatggtaataccaacactctttttagtcgccagttccagcagaacagctgcaacaatgcctttaaaccgctactcaggcgtcaaccccactggaattatgtttcaccaagcacctccttacatccattcggaaccaccaaaggaaacttactacaaactcagttatccttatccgtcagcaacagttgtgagaaccttgaaaggccatctctattaccatagcgatgataccgttcaacaaaattgtaccatgcaatattcacttatagctgcttctacaaagcacgtgtgtggcaaggtagtttacactataccagcccatgtctcaattggctcacttaaactgttgcttgtccacccggatcaaacaaatttaccatttgaactaccagtttcagatgaagtccgtctttgctacctcacaaccttgaacgcaccaagatgcatgccctctcaactagccatgtcaaataaccaatttgccgctgtaagccttgttttgttaataacattagtttctttaattaaagtttatataatgttttttactgtttttaaacactacacaacaactgtttttatacttgtagctgtgactactatcacaatgttggtgtccttcttagctcctccacttctcatagtcgttcttctttcactagcatggctatggtacggcaatacaattgtattgtgccatatcatgcttttgatagtcttagtcgtctcatggaaagtggctgctgtctgtttcatcttcgccttattgtactttggaaaatgtgctatgcttagcaagaacattaaatacgtacagggtggagttaaattttcaggaacctttgaagaaatagctcagtcaaccttcttcattaactacggagtagcttgtcagcttctggaacatactggacagacaattgaggatataatgcaacttagaaccgcgggtggagccccagcaaggcttgcgcgctcaatatacgattgcttttccacaaatgcctctgtcttgtacagtcccaggtcattttcaccacagtcacttataacaaaatatttatacccaggttcgatccctgtcggcagagcccctgtcttattaggcaaaatctccggcatgacttgcttagggcgtgaacagtccacctgtttccaatcatcagctacaaccattactacgtgtacccatgctgtaaatactgctggaacattcatgtctcaaattaaatgtgttatagataataaaatatatacagttcaacccgagaatataaccataaccggaatgaaagctacatttgaagttgaaggactacctccattcaccaacgatgtaacagtggccccaaagccgctgaagcattacatggatggaaagagacaccttgttctctacactaaaagtgagagcatagtctactcttcaataatgtggccgactgaaaacggtttattctcgtcatcagtttctgacccaggagattcaggtgcaccctacttttcagacaatgtcatagtaggaatacaccaaggtcgcaacgaagcaaccaacaatcctgccattttagcaagtggtatggatggtgagtctccctgtgtaggttacgatgaccaatcatatggccttccacttcaagaatatttcactcacattgtcttatcaaataagccaagtgactttggtgctccatctaacgtggcgccaaataaatactacaacaaaaaatcatttgaacaattagctgacgaagataagacttatttaaatagtttatcatatcccctgtcctcatctaattattgttactttaatagcttcaaaacccaatcaagcacaacaatgctcgacaacgctgaagttattaaatatgtagttttgcttctcatgatcttggattatttcttttcaatcatttgcgaagatgctttaaacccagcatcttacgctatgttagttatcgttttggttcaggcttttattacaaaaattacagttttcagaacaggtatctatatccaggcagccgtttttcaagcatttattgtacctatagtcagtcaaattacattgatactggctgcagatactgcaagaagttttttaacgttccacttttttgtacttgctgttttgacatatttcgttctttgccgtattgctgtagatttttggcgttccatgtttttgctatttttgacaagcgtctttgcaaccatcatatggactacaaagaatgacttcaatattttacatgaaactggcgtggttctaacacccacggcagaattagctcttatagtagcttttacttatataatttatgcttcatgtatgttaacacctgtaccactgtatactatttgtgttttcttttcatttttatcaaatgctccactctaccttgccgtcctctcattcggcattctagtttctttcaaaacaaaccaagattttggacgtctagtggataaagtgttttctttaaatatgctctatgaataccatgcttaccaaaactatgttattcaaaactcaggtcaacacccaggattttacaggtcactctttgctttttttatcaatttgaccacccaaccaaaaacaacatacaaatgtttcaaaccccagacagcaagtggttacagagtaatatatcaaactcccactacagagttcaataaatctctgcaacatgccagtatcacaaaagatgacaactccaaccatataattatgtttgctgacggctcatctgataatctcaattgggcaaaagaaatggtcgcaaccattcatctaaccaacccaaatttgcagccactcatcattggatactaccacaactccatggacgtcataaccaagggaacttacatgcaacatgaattcataaaaatgccagctgttatcttaactcaagatcctctaactgaaccaatcagtcatttagcagcagcagcatttacttcaatttctggaaaacctcaggcacagaaaaacaacgttgtttcaaactccaaagcgcgcataaacacagccgttcacgacgctgtcgaaagcgtttattcaggagaaacatacgttgcccccaaacctatagtctcaggaaaaactgttgtagagaaaccattctctacaaccgaaaccaccatgtacataatgcgtggtttacccggttctggaaaatctttcaaagttagtcaattagttgctaaagatccaaatttagtcgtagcttccgcagaccactttagatattcaaatgacaaaactggaaaagccgtatacacctacattccagaagcaactagttctgtacatttacaatgtcagaatagagcccgcaaagctctagaaaacggccaatctgtgtgcattgataatacaaatctaacactcttagaaatgagaccttacgtcttattagcccgttcttttaactataacattgaattcatacactcagactctccctgggccttaaaccttgacctgttacatgctaaaggtgtacataatgttcctagagcaaagctcgtaatcatgtatgatagattctttgaccgtgataatcaaatcgatgcagacagtcttatacagtatgttattgaagcaattgatccaaaacttgttgctccaatcatgaaccgtttccctgccgactgtgatcttatccttcaatctgccctaacaccagaccttgaagtattaaagcaaaactacgacagagcaaacgcaacataccaagatgtttctttagatgatcctccggctttaaaggcagcacgtcgtgctatgaatatagctaaatctgaatatgaggcaggcgaagcaggccagcgtcgcattgagaaatttttagaaagacaggatgtagcagcactcaaccaaacgctcacaactgtcaatcaatctaaattcatagcagcgatccgttccatctacctaagcaccattagcaatttgagactaaaaacccgtcatatgggtgaaggatcatatgcagttacatcaggtactaatactaccgataaagttttagttaacacgccacaacgtatgactagaattgaagatggcatttataagcttgttgcaaacggttttgaaatcacaatgtgcgacggcagcaacttagccggtgttacttttgaacaggatataaatcctagcatgtacccttttgtttttacattaatgtcaaatatagctgtacctgttttaacccgccaagcaaatgttggctatcttgatatgtcaaataaattcatctgtaaagatggcactgttcaatttcaaggtgtcatctatgcctaccacactccatcaaatgagagtgctgacttcaaagtaggcaataccagttggaccctccagaaaaacatcaatttgactgcttttattcctgcaattcataaaactgcaaccttcgcagcacaatcagtgttcttaggaggactacccatggaagagcaccaagccttttccgacacacccacagcctcaaacaaatttaaagtttttgtttcatccacagtctgcgcctcaaccgtgtgcaaagtaaatcataaaacttatgtacagataccagatgacattcaagatccttttacatatatgcatcacagcgtttgttcacacaacaaatttttatcaaaccatgaaaccagatgtcaaatctgtcctttaaactgttanagcgcaaatccgtgtgtgtctacggcttgcgctctatttgataatggcacattacctcggtcaacacattatattaatgttagcaccacttcaaatgttggcttgtttaaggcagtaaagaagtctactcgtcaactaaacattgacggttttccttacatgctaaagcaggttaaagacgactcagaacttgtaagttctcttaaaataggtctacctaatatcctcccacatcacatggtggaaactaagtcaaaaacataccttcttaggggccccacaacggcttactcacttggcgatttatgctacgcactctttaatggcgactttgattatattcgcgaaaatataaactctgatttcgttttggaccgtgaagccggaatgcctgatacagaaacacgtacgtggctgttcagcattttaaactttgcagtacctagagtgtgtgctataattgaccagatgatttctgagaacgtcttctataaactgactttggataacttagatctatacggatcactctatgattttgacgactatcctactgaaggctttaacaggcctgatgatgtgatacgtatgttaaaggagatatggtccttctgtagacgtccactacctgccgaccttcttaaataccatgaagacatcggtgcagcagccactcaagaaatattgctgcatgcacccttcattgataaagtttgtgctctaaatgacagattagctgttgttgataatagagcaagtcaatactttttctgtgaagaagaaggtgtctttacccatatttacaatccagtctacggaactttagcattcgataacaagttgatccaatcaaaggatccttcatgtacattacagcgcctcattactatacaaggccctttgtctacgaatgctagtcccgtgatctctatttctgattccactcatattgccaacaatattaatccatctaaccaaaagacaacaccgttgtactacgatttggaacttgcgcaagaattcattgacgcaggtttaaatattgatggcgtttccaactacttcttctatggaccgtctagagcgggtgtagtgtctgatttcttactatatgaattccaaggaactcaatggtttgacaataacatgctgcgctctctttattctttcatattgaagaattcagagtgttacagaacaacagatcaactggactttagaggtggaaaaccccgtaaatcctcaatgggacatggtgttactggctttaagcaagacgtcgtgtacgctgctttaggccctgatatgattgaaaccttgtatgaaacggcaaaacaaacaccattgccgttttgtacaaaaataactgccaagtatgcattaacagcaaagcctagagctcgtacagttgcagcatgctcctttgtagcctcaactatttttaggtacgctcacaagcctctaactaataatatggtctcaaaagcacagcagggtttgggttattgtttaattggaatttctaaattccacggtcgatttaataaatttgttaagtctagggtaggcactgtcgaagactttaatgttttcggtagtgactacactaaatgtgaccgtacatttcccttagctttgcgtgctctttcagctgcccttattttcgatcttggcggccatgacccagacaactgtctttttattaacgagcttaatgcatacatgctagacattgtttcagtcgaagactcctttgcaaataaaccaggaggtacttcatcaggagatgccactacagcatactccaacactctgtataactttgcagtccactatattatcatgtggaaaacattcttgacagtcaatgacccttctaccaaggtcatacgcagtgcagctcatcacgccctaacaagtggtgacttctctatgtacaatgacatgatacaagacatgttggatgtagactatacactcaacttcctctctgacgattcatacatctgttcaaaaccaagcgcttttccgatctttacgctcgagaactatccttctaaactgcagtctatactccacacagcagtagatagcaaaaaatcctgggaagcaaagggtgagattaaagaattctgttcctctcacatagtcaacgttgacggcgactaccactttaaaccagaaaaggatagaatattggcttcattgctgatattatcgaaaatcgctgacatggacatcttctttatgaggttcgttgcgttattggctgaatccgccgtatatatacgcatcgatcctacattttggctggccctgtttggtgttttcgaaaaccgcgtaacagcgtttaaatctgaaacattgctctcacctgttcctgaacaactcatgaaggtggctttttatgaatcgcttgtctttgccgacgtggatgctacagccttatatggtttccttgatggttttaaaatgcaaagtcaaactctccacccagacggtgttgagggttttgacaagcaaagtgaccgagtaaaacactgttttgcttgtgacaatatatcagttggacactgttcgatttgtcccgttccccttcctttgtgctctttttgcttctatgagcatgctctgctcaatgaacattatgaagcttctggaattgcgtgtgaatgcggagacgctgacattagacaacttcacttaaaaataaccaatcaaccatcctcgcacaattttatctgtgctgaatgtcccactgtagctatgaagctgccaatcttcaactctttccaaggaaaagtactgcttccaatgttccgtatgaatacgccattgccttcctcagtctctgtaattgttgatgtacgttccaacccaaaagcacctaagatgctgtgggacgacgtccagaatttcagagaaaattgtactaggatagcatacgaatccgtttcgtgtgctgaactagctagggaggtggtttactatccatatgaagtgattgaatccaaagcaggtcaagcacgacttagaatacagaactttaaatgttcaccaactacttatgttcagttctacaaagtccgtcaaaatggaaagtattgtctagtagccaaagcaactctaacgccggcttttgaaaaccaaacagacattttctccgtttttcaaccaaacaacttttcaccttggaatacatcatcagtgtttgcagtagaacaatacgctgcaatataccctcccataccaaaggaaccagtcaatgctacgttcgtcttaggacctccaggctgtggtaaaacatactacatagccaaaacgtacttttcacaggcttctgagacatgtccggtcgtatactgcgcacctactcacagattagttttagatatggacgcagaatatagtggtgtagtttcaaaatctctctacaataatagagtgtacaaaaatccagcctacaaaacaggcgaaccattcaaattatgtttcaccacacacaacacgatgccagttcaaaagaaagcgatcctcattatagatgaagtgtctttaattacaccccactctctattttcgatcattggtaaagggttctatgagatagtactcgtaggagacccttttcagctctcggctgtttttccaggttttgttgtcaatcacacatatgacgggttttacatccgccggctagtaaataaggtcaaacacctaacagtttgttaccgttgtccacaagaaatcttggacatattttctaagccctatcatgatgttgggattgacctcacaaccggaaataccaatccaggaaaggcatccatttatacactaaattggcttcaagcagatgtaggtactaaaaatccggacaaactcagacaactctttgcgcaatatccaggctttaaaattatcaccaactacagatgtgttgttgatgcagctaaaagttacggtattaacgtcgaaaccatcgactcatcccaaggaaccaccggagataggcatctggtggtaatttgcggcagtaccaacttttctaaacttttaaacaggtttatagtagcagcctctcgttcaacaactgaactagttatagtcatgttgccagagctttacaactatttaacagagacgtttaacttcaaaccgttacaattgcaaaatgtgcatgtaccgatcgcagtatcttctacagcattctgcgatatagaattttatcactttcaaaagaagttctatgttggtgaaataagcgtaagcacaagtaccactatgacatgtcagttgggttgttatattaatggctcctacatgctcccacctgtgcttgaaaactctgaagaccgtctctacgttccttctagatggagacgtatgataagaaaataccctactgaatctatgcacatttccttactggacagacttctgaggcacattttattaacaactactggagaaattcatttcgtaatgttctctgcagacaatgatctcattgcactggatccgtactttataccacccactctatgtgagtgtggtagtgcgggtctagtggaagtagacatcactgttttctgccgcaattgtttgcctaaagatggtaaagccactcgtttggtaaaaccgtctacactagatgtccagactgaaaaactcagacttgcaaaagttcatgctaaggtttgtaaaatcaagcatggcagtgctcacaacgctgatgttgatgctattatgactcaatgtatatatgctaatagcttaacattcacaccaacaacccaactagttgttaacactgatgagttcaccttttacatgctacctaggccgtcaaaccgtcatttgagaatcattcataagaacgacaaacgtttctatgctatcactcatgaagaagaagatctcttctttactaacatctcagcagtggtagacccaattcctgcaaaattcaacattgcacactctacaagcttcctcaccatcaaaagtggttgcgcaggtaataagacttgtaccagatgctattatttacacttagcatacacggaatttgtttctcaacacaagtatgaaccattcacttgtgtgtcttttaagatacggtttgacttttcacaattcactgactcagtagatactttcctccgacaaggcttaataacctttcatccggagatgaattcactgcaaaaatcacttttattagcagtggataaggtctattgtgataacttcacctcaaacggtagaaggtttagactttacgacaacaatttggttaaatccataatcaaaggttcagtggctcaaaactccatcatcatgccactcgactcagttttacacgggttgaacattgatttcacagtcggatgtgccgtagataacttttcctgcaaagaagcagcgagtgttaggtactcagaagtagtactttccatcaccaagttgcccccaggcacttgccagttatactacgtcatatcttacggcctgaactctcccaagaccacttatgctggtcaccaattgttcgacggctttgagactgttattgttgttaatcgtaaagataaacccccttacgtcctcacacagtatattaatgatgttgtagttgcaatgccagagtccctcttttcaacaggtcgattctacagagaaaaaccatatcccgtccttatgaacgaggatttaagtggcttaaaccatcacattttctctggtgactatacagacgaatctcttacattaggaggtgtccatcatatagtaactttaaacacctatgaccacaagctcaactatatccaaacgaaagctacatgtgccgcctcagtttcaactggcggacgtggtcataaaattactacactgtttgacgttcatgcaaatcaacttgctgatgaaattaccagagttacatctgttgttacaacacagtctaaagttattaatttgacaatagattatcagcaagttccttgtatgtactggtcttcaccgaccggcataagaaccttctaccctcaggctgttagactggacgcaaagtttataccatactacgtagaatatcccaatattctaccggcagttgttgaagaccaggtgtacgatttgtctaattacaatcaaccacctttaggccaaaactgccctgtaaactttcacaagtacgtccagctaactcactttattttagatcatgtgaaaatccccgaaaagggtttgatatatcatatcggtgcagcaggtactaagcaatgttcacctggagacttaatattggaacaatttttcaataaatccatcatatactcaagtgaccttcttccttaccaatcacctgctgtgcaggttgcattggatgtaaggttttcggcttcactcatcatttcagactgctattcgaaagaaccgcagcctgatttgttgagtaagttgattaacaaactagtgtatggtggaactctcatttttaagaccaccgagactttcacatgtgacccagccttttatgttgctcattttaactgtataaagttttttactgccgctgttaatcactcatcatcagaagtttatattgcgttcatcggaaaactccctaaaccaaacaacaactttttagcctcagactatttccagagattaactcaacatagaaataaagtagttaaacagccttacgctcacacatgggacacatcttttacgtacccatacccctcaaatgttcttcaagttagtcgtaaaaaccttttatatctatttgaaaccagaggagctgcagtaggtactttgatttttgaagaaccatcaaaacctgctgtaaagatacctacaaagtgtcaaaccacacaaccctcgtgtgtcattgaggttggtaaccaatacgattgttgcattcaagacatcattaccctcctcaatggaaaatccttcacagtgaaggtgcccaactcagaatccttactgcgcgatatctgcacacttgcgcttagccagagttattccatcaatattcgcggaaaaacactctacacccttagttccctacttagaattaggcaacaatccttactgttttacggagagaaggtcaaaaaccctcgaccccgtaatgtcttgaacaaatataccaactacctcaaggcaaaagtgattaggcattacaccaagcctcaatcaacagttttggacattggtacaggaaaaggacaagatttgagaaaatactcgttagcaggggttaaatccctcacttgtgtcgagcctagtcccgagtctgtgactgaactttcaataatagctagtccccttgatatggagacacacacagttatgagttctgcccagaaattcgagacctcgctgacgtttgacttggctttctctttctttgccttgcactatgcattggatgacgtttgtatgtctgaaacactcaacaatgttttttgcaaacttaacagtaattcacagttgatcttagtagttccaaatgctggcaggatgcaatccataccttcccttggtttaacagtcactcatctagatgatgataaagtttggtttaaatactcagactatatagactgcgaagaaccgttagtagacaaagaaaaactacttacgtgtttagctacatatggaacaattgttactgactcaccattctatgacggtgcaaacaaaatcctagaccaaaaatgctcatccatgtatagagcatcgacagcccatctaaatcccgatgaaattcaatatattaatatgtatgatttaattgttgtcattaagaat

SEQ ID NO: 2 - DNA, 4173 bp, Unknown
  Fish coronavirus ORF-2 nucleotide sequence
atgttcgcgctcgttctaaccctcacaatagcttcggctattgcccaagatttccccgcatatgacccgtgtcctacttgctcaacccccggtaataaaataccggctccgagcacagttgcccagtattcaacaaactacggtgcgaacttctttaccgtagtctttgatggtattatcttcaaccaatttagggagagttattaccaccaatgtagaccaacacctgaatactgcccagatgcaatcaattgcgccttaaacagaacaggcgcatcctgcaaacctttcgcaactggcccgaattcacaatgtcagaacagtttcgagggcaacatcgacatatgtgcaacatgtagccctctaaaacaagaaactccattcatctgctacaatagatacgggataattatatacccgacagcagatatcgttctctccgctaggtttaagataggctctttttcacccaaggcttgtgataactacctaaacgacttaaattgtgattcaaaaacggcaaggtcatatgtcatttcccgaccgcagtctttttcactgcaatatcctaactcattaggcccctatcagctgaaacgattttctcttgcaaaggagatcgttgacttacgtgctggcgttttaacctcactcccaaaccgaggttataagggtagaacaacatactcttatcccgtcactgcactctcactcttggctcgttccaaagtggctgaagccgacaaattcttttatatcgaggctaaaattctactgtacgcttggtcacagaaacctcaaatccgctttctaggtgcatactgtcccacagacgtgtcatgccctgattcaactgccctcggctgttgtttttccggaagtggatctgagttttactacgcctttcgccagtggtactacgcaagcctgggtatggaagacctagtcgactttgataattcaacagtcttaagtctctcgcctgatactcctcaaattacacccgttgtgtcttattttctagaaaaagttttacctttgtttaaatcacatgtacccggacgtgttttttactgccattcacttatgtctaacggtgtatgtacttttgaccatgttgttgtaaatattaatgccgaggccgtcttttttgacctcgaagtagacataggcagcataattgctgacgcatatcgcgttgaaaggcctaatactttatgttatgatacaaactgtactcttgccacaagcaggaccactgagtataattacgctgcttatgttgtttatatactcttcaatttgtattctagtaatcgcattgcgatagatttcaacacacactcaatcttgcaaggattactacaacacaatagcaactaccagactgctaatttagactatctgtttgttggagcactttttacaggtacttttaaacatattacaagcaatcaagcttacccagtacctttaacttatccaattgttaagacatatgtagggccgtcaaaccaatactcaatgtcaaataaactgttttcatatactcacaatttgacggctcaagcccattcaggcatatgtaactctttttactgttataaaccacgttttgtaccaattgatgtttttattcatagtgctttaacccctgacagcttgatggaaacagaatcttttgtttgtgtctctttgcgttcaccatctgcaggatcaacatccgcaggtagtttttatttgcaatgtctcaattcttccatcgatttgcatccaggttcatttgtacccgtttcctcaagtccagagtcttccagccgcgtaacagctgagctggcttttaatactagaaatggtatattttctccttgtcttaacggtacatgtgtactcgcacctactgacccaattgtttttatgcgtcagggtgcctggtttacaaaatctttacactttgatgtttcaccatgcaaacctatgcattttccagacatagatatacagcccccaacatacaatgtctcctctatcaagatggacgacaatgctgtattggttcaagaccttacttcgggtttagtaattgaccacaatttaggctccatactcagaccgaaaggtagagctttggaagtttcgtattatgctcactccattttacgttaccttgaaccggattcttgtctacctgacaactttcttaactttgtcacttgtttagactatatctgttcagactcgtcaccttgccgtgctgccgcaagccagtactgtcaggcaggcatttattttgagtctgcatttaataagtctaggtattctttgcttaacgcttacacgctttttaacacaagtcttcaaaccttattgcctgagacttttcttgagatagaagatgatgaaccccatagcagatcaaagagatcaattgatactacaagcaatattcgccctagtcaattgcttgttaatggacgtattccgtctacaagttcagcttttgctgttaacgtcgctcgtggtcgaggaacgattatgcctcgtcctggaactggtggcatgggttcgtccttttctgctgtttctaggtcgggtagtatttcttccttatcctcggttggctcctcaacacctttgatctctaattggagaacatcttcatctcaactcaaaactctcaacctcaacattaacactaaaattcctaagatttcaacaaagtcaggttttgccagtattacatctttgtttgcttcaggtttaggagtcgtcgatctaggtctatctattttcaacatgatagaacagcgtagagttgctgagatcactcagatgcaaattagccaactggctgactctatagtgtatcttgctgatgtgacatttgaagctatcaagaatttggaactctcggttaactccttgggtacgttcttatcggaattttccactcagatgtcgatcaccataagccaaatacaatcatcatttgaagagcagcaagatgctacaaatgatgcgttgtactacactaacgctgctgcgtcataccaagcctccatggcgtatgtcatttcagagttaaacgcaatatctctgtctgtcactagatcctacgactcttacaccagttgcatcacttctggcattaatgggctcattacaccatcatgcttgccagcccaccagttgttacagttactcgacaccgttatcaattccacagcaggaacaggatgccgtcccatctacggcagagaagaagtggtgaaatactacactttacctctaatcaatcaaggttattcctttaacgggtcgattttcttcgtctttaacattcccatcacttgccagggaattgccggagatgtatatgaagtagaaccacctatacttgtagatgtaccatcaaagactgctttacgcatgattacaccatcaaacgtagtcgcaacacaagcaggattagctgaattagatttgcgtcattgcgaaaggtaccataacgagttcctatgcgattcttcagcattcctttctacaccttcaaaatacatagactgtttaacaaacgcaactgactgttctttgcaattcatcacacaacacgttccagatccttgcgtttacacatcgccagcttctttatattgttattattcacccatatgtgatcaatgtcacatagtagccggttgtaatgaatctcagcagtacaacttcacttctgctgatggcggcgtagtcttttattccatacaagacagagactgtggccacttcccccacatcactgttactacgcctgcagccatacaagaagacttcactgtcggaccgtatttaccatcgctgccaattcacaccgcctacgtcaatgttacctggaatgtaacactaccaggaaattggacctgggaaaatatcaccctaacagccaattggacccaacacttcattgagatgaaaaaaaacatcacaatgatggctgaagaaatagataaccttaccaacttcggtaaggttttagttggccagctaaatagctttttatcatctttgtttaacataccattaggtttgatgacgttttgcttttctgtagccgctttaggcctgtccattattgctttacttgtgttatgttttccacagaagccacataaatta

SEQ ID NO: 3 - DNA, 780 bp, Unknown
  Fish coronavirus ORF-3 nucleotide sequence
atgatgtttaccctagtagtgctttttaccctcctcggcctttccatggcctccacagagctgaatttcgatcctactctacccctcccctctcctataaatgccctcgtcgacattttcggaaacaacagcttgtttctcaaagagtccctgctcggcaaatccaccggagccgtctacgcatacttgtacagcagtgccatctctctcctgctgctactttgggtaactgtatggagtattgctacttcacactttaacgtaactcgcattccaaccatcgcggttctcactaatgcgagtatgtttttgctgttggcatcggctactgttacaacctggtttctcccaactgtgacgaacgtcttcttttatacactcactgcgctgttcaccttcttttcctttgtgttcttactgtggttggtttactatatgtttactaccattagggcatatcgaagggtcggttcatggcgcgttgtgtttaacggaaaatattctctacttgctggaactcaggctgtttgcctttgcagacccgccatacatctggttctaaccaaaacgaacacagatacatactggtgtctagatggaacccccatctacaatgttgacttactacaattagttggccccaaaggattatatccttacaaaagaatgactacaatcactgcaccaaaaggcacaaaaacatctgctgccgtttacacccttcaaaaagaagaagtttgtgctctctcagaaatcacagtacataatgatactgatttt

SEQ ID NO: 4 - DNA, 603 bp, Unknown
  Fish coronavirus ORF-4 nucleotide sequence
atgtcttacccggtttactacgaacagcgtcgttattccccccgccaattcaacaatggcggagggtataatcctacacctcaacctagagtagttcgtactaatcctggtaaccaagcttacaacccccggcgtaaccgaaacgccactccgaaccaacaacaaatggttccttaccagcctcagtatcaagcacctcctcagccaagggtggtctatgtagatcgccctcaagaacctgtagtaatttacagagctcctccacaaggaaaaaaacaatcaggcaaacgccacacagcagaagaacgctggtatcaaggcgaaaaacctgtgcagaagaaacaggcacccaaaggaaaatcaaagaaagcagcaacacctgctaatcctaaaaagcagcctacacaatctgacaaagttcccatcgcctacccagacaatcatcccttccatgacctcgcaccagctgacatccgcgctttcaaaaagcagctgatccaaaatctggaccttggacatggtgaaatgaatcaactgcggctttcaatcgatctgttgcccatcaagaaaccagcaccaacaccagcggtgccagctcctctg

SEQ ID NO: 5 - DNA, 339 bp, Unknown
  Fish coronavirus ORF-5 nucleotide sequence
atgtttacccttgtgcttattatcctgcttagtttttctatggcttttaatgcttttacatttctgctgttattattttttacttttaagtgcattataacccgcactttagtcgtagttcccattgactacccagaaaatcatcctttcaatggcctctcaccagaggaaatcatcagctacaaatcacagctgatccaaaatctcgatcttggacatggtgaagtaattaaacatcgattctcaattgatttacttcccctcaaaacaacaagcactcctaccaccagtgctattttatggaaaaggttcaaaacctcccataaagaaaacaaccac

SEQ ID NO: 6 - DNA, 24613 bp, unknown
  Fish coronavirus genome nucleotide sequence
  misc_feature (2748)..(2748): n is any base
  misc_feature (2749)..(2749): n is any base
  misc_feature (2750)..(2750): n is any base
  misc_feature (2751)..(2751): n is any base
  misc_feature (2752)..(2752): n is any base
  misc_feature (2753)..(2753): n is any base
  misc_feature (2754)..(2754): n is any base
  misc_feature (2755)..(2755): n is any base
  misc_feature (2756)..(2756): n is any base
  misc_feature (2757)..(2757): n is any base
  misc_feature (2758)..(2758): n is any base
  misc_feature (2759)..(2759): n is any base
  misc_feature (2760)..(2760): n is any base
  misc_feature (2761)..(2761): n is any base
  misc_feature (2781)..(2781): n is any base
  misc_feature (2782)..(2782): n is any base
  misc_feature (10029)..(10029): n is any base
gcagtccaccaacacaacgtggctctctgcttacctgtaagggcacgcccttatgctgaaataatcattcaggaacacctttttgtttcaagaaatagtaggagttttaaaccatccttctatcttgttccaacaaaggagccgcaatcaaacactctaacccttcatgggggtgattttgatgcaaagcgacacttgggtaagaaaccagtaaagtgcaatattcttgacattgttgacaaaaaccacacaatagcaatgaaaaaaattgaagatgctttaggaacacttaaacctgcctttaaggcacacatagactctctacctcctttcatgggaaaactagccacaatgctagctgagacaagacgaggaaaaactccccctcttttgatctatgtcatatcaacaattttggaaacgaatattacagttcattatgtcagccacacaataacttcataccattcttcaaacgcacacactcacactcatgaattcgactcagaagattacactcctgacaatcagattttaactaaagtcaaccgacatggtaatgaccttaatcattcatatatccatggcgccaccaatatgtacaaccctgtctaccagagacacccaccaaacatgtgctacatgcttacgggactgtacatgttgtcaggcttacaggaattgtatgccatggctgaagacaacttgacaacatgccaaatcaaccttctcagatgtctgtttgatttgaatcaagatgaatttgatgtagattacacatttgtaatatacactcctagcaaatctcaggagtgtgccttcaaatatcttcaggagatagtccaccactgtgaactcaccatttttagacatacaacaacaagtgtcttcagttgcaacaaatgcaaccatgtggaaactgtcatttctagttgttctttgaatcttgtatatataactgattctattgaaaaagcatttcaacccactgtagaagctaataccgactacatgtgtgaaaactgcggtctacgcgaccataaacttaaaacaacagtaacaaatccagacctccgtttggcacaactaaactatccaacggattcaaaatacactatctttcttgatgaacaagctccttttgtcttccattccattgcaaaacacgttggaactgctaattccggacattggagtgcattgaatgtaaattcagatatgttgtcagactctaacgagcgacaacattattatacaacacctagtattgttttgcttgcatttcttcctgaggaagagctacagaacattagaaattcatcacctcttcaggaccatcaaccagatgatgttgaagacgttgaatctcccctaccaggttctatattttatactacagatgacattttttctactaagagtctatccatagctcactgtgtagcccgcgactttcacatgtccggcggcatagcaaaaattttctcagataaattcggctctaaaactttcttgaaatcacaaaaccccgttataggcggtttttccattttacttagagagtgtcgtgacatgtactacctcgtcacaaaagagaaaacttcagataagccgacataccaagaccttaaaaactccttgggttctatgacagagaatttggttcgcaagaatcataatactctttcaattccatatataggctgtgggattgatggtttacaatgggcaaccgttgaaaaacaagtcaaagaaattgtctgcgctcgaggtattgatgtaacagtccaccacctcgaaaatgaagttaaacacactccagaacaacaaacagcttctgacaattcagtcaaattagttcaaaaactctttacagaaacacctcaagctatacctatagttgttccttcagatgactcagacagcgacatagatgagtcagccgatgtctttttaccaggacctgaatctgaatctgattcaaaatcagaatctgggtcagattacgactttaaatcggcttctgaaccagaagatgaattggagcccactccaatctctgaacttgaactaacaccagcgtcaagtttaactgtagagtccgatgacaatccagacacaagccaggaaacattaccagaatcaaactctgaggaaaccaaacctgagcaaacacctgacacaacatcaaaggtgtcctccgattcgaagcttgatccacaatcagaattagaagaagaactggccaacaaaccagaatcggcttctgaaccacaatctgagactgaatcgagttctgaatctgaagaggagcttgagccacaatcagaatcagaggaagaaccggccaacaaaccggaatctccttctgaatcacaatctgaaaatggatcgagttctgaacccgaagaggagtctgaaaaaccatcggagtctgctgaaacagcaacagaggatagcccggaaacaacaccggaaacaaccttagagttaaccacacaactcaaacctgcttcagaatctgacgacaaaccggacacaccagcaccatcaccttcaccaattcaaccagagaaaaacttggacaccacccctgaacaaacttcacaaccaaccacacaactggaattgacgttagaaacacaggaacaaccagacactacaccagaagtgccatctgtctcagaagataaaccagacacacttgaagaatcttctgaatcaacaccagaactctcagaattggannnnnnnnnnnnnnccaccagtaccaagacctanngaaccagacctccaacacctagaccaagatctgcacgtggagctaggactagatcctgtgctggcacacctataccagttatttttgatattatagataatacaagccagcctcaagttccccttgacttcccagaagcactgcaagaactgaacaaacctagtgaagtaatcccagcggctagtgaaaaacctgtggaaaaacaaataatccatagttttgtaagtgtcgaaacaccttgcaaacccaaagccactaaagttacgaattatgtagctgcacaatctaatgcaattctaaattgtattaaggctttcattcctagcaacccgctttcactattcaatagaaaaccagcttttagaaaaataatattcactgaagacacttcagaaccagatagcgatgatgatgattgtgaatacactccaccaacatcaccattccctgaacttctggcattggtagatgaagacattgaagtagaacaaactcaatctgtaattccaaaaacagactctgcttcaattgtggaggatcttaaaaaacaagaatcctctactttgtcattggacaccaacacatcgaaacctacaagctctccgcgaagacagcctagggaagtagaaagtgttgatgaatccagtgatgactcatctaaaccaaaaacaatttcaacattagacaaacctgctatgaatagtgacacgaaacctacagactcttcgcgaaaagagcctctggaagtaccagttactacatctttaagcacccctgctaaaaaccaagataaaaaatcttcaaaatctgcaaaagtaataaaagactattctttgacccctaacacagtcaaacagcaagtctattcactctacggtgaatcagtagatgcagttaaataccttgttcaaacatacccagacagggctaaacaaacagctggtattgcttatttccttataactacttatttaatatggaccatcggtctcataggagtaccaatggcctttaaaataccaatgtttttatgtcttttataccaagttaatggattaaatatagcaccatttgttactaaccaaaagttacaatatgttgcatttccactttggtataagctctatgaagtaatatcagtccgttttgtggcgaatatagcacaatttattgttaaaacaccacctatagatgttttaaacaagctaattcgttctaataaagacaagccagtcaaattaacaccaaataaacatactttgatgttaattcatgacttagctttagagtctgtcgacggaaaagaaaaccgctattataatactgatgttacaactttcacaaaaaggcatagcacttctaatatttcatatgttctaaagtctactttaatcaaatatgtcatggaccattgttatgtaaatattgcagtttttactttagttagatacttaactttattagtgtttattcaacatttctctaatccttatgttcttgaagcaaatagccaatcacataccgttttacagtatctgttttcacacttgagaccatttggaaggcctttgtgcccaaccctcaatgactacatgacgacagcaacaccacgcgatgcacatgtacaagcaggttctcacttcagtgaattttgtgttcctattcattatacaacaccaatcattaaaagcacaatggcagaaccatcactttttctactttttaacccagtcttatggcctttggttatggttgtatatttttatcctccaatgatgtttatagcaaatgcagtcgcttattcatgccttcctttagtggtcttgttacaatggctttatgccatgtggttttcttgtacatgctatggcaccaaaagatgtgccaagcatttgcataaaaatgaagtggttaaaccaatggaatccacttcaactaagaaccgcatgacatttactccatcaacgaccttttgtagtaaacataacttcttctgtccagatgcaccacatataatgactcttgcaatggctaggcaacttacaaactactacaatttgacagatacagtaatacctgacatccaggaatactcccacgagaaccctactgtacaatttattcactttgatccacttaaacacggtgccgacacaattttggaaccaattacaagcgctagcgccagttcaattgttgcatggtactctctcctctttaatcaaaagtttgtcctttcacattatagctacagaaccccagtagccgtagttgacaaaccagaggaaacagatggtgatgatacaaaatcattagcatctgacacttctgataactttgagtctattagaaagaccaaccataagaatcagagcaaacaacagtttaggccaaacggtcaccaaagaccaagtaagactttcaaacgccattcaagaataatgacatctgaacagaagaacagcttaattgaaacttttaagggtttaacaaatggcacagcagccatcccacagcctttaatcatttttatttgggttatccttatggtaataccaacactctttttagtcgccagttccagcagaacagctgcaacaatgcctttaaaccgctactcaggcgtcaaccccactggaattatgtttcaccaagcacctccttacatccattcggaaccaccaaaggaaacttactacaaactcagttatccttatccgtcagcaacagttgtgagaaccttgaaaggccatctctattaccatagcgatgataccgttcaacaaaattgtaccatgcaatattcacttatagctgcttctacaaagcacgtgtgtggcaaggtagtttacactataccagcccatgtctcaattggctcacttaaactgttgcttgtccacccggatcaaacaaatttaccatttgaactaccagtttcagatgaagtccgtctttgctacctcacaaccttgaacgcaccaagatgcatgccctctcaactagccatgtcaaataaccaatttgccgctgtaagccttgttttgttaataacattagtttctttaattaaagtttatataatgttttttactgtttttaaacactacacaacaactgtttttatacttgtagctgtgactactatcacaatgttggtgtccttcttagctcctccacttctcatagtcgttcttctttcactagcatggctatggtacggcaatacaattgtattgtgccatatcatgcttttgatagtcttagtcgtctcatggaaagtggctgctgtctgtttcatcttcgccttattgtactttggaaaatgtgctatgcttagcaagaacattaaatacgtacagggtggagttaaattttcaggaacctttgaagaaatagctcagtcaaccttcttcattaactacggagtagcttgtcagcttctggaacatactggacagacaattgaggatataatgcaacttagaaccgcgggtggagccccagcaaggcttgcgcgctcaatatacgattgcttttccacaaatgcctctgtcttgtacagtcccaggtcattttcaccacagtcacttataacaaaatatttatacccaggttcgatccctgtcggcagagcccctgtcttattaggcaaaatctccggcatgacttgcttagggcgtgaacagtccacctgtttccaatcatcagctacaaccattactacgtgtacccatgctgtaaatactgctggaacattcatgtctcaaattaaatgtgttatagataataaaatatatacagttcaacccgagaatataaccataaccggaatgaaagctacatttgaagttgaaggactacctccattcaccaacgatgtaacagtggccccaaagccgctgaagcattacatggatggaaagagacaccttgttctctacactaaaagtgagagcatagtctactcttcaataatgtggccgactgaaaacggtttattctcgtcatcagtttctgacccaggagattcaggtgcaccctacttttcagacaatgtcatagtaggaatacaccaaggtcgcaacgaagcaaccaacaatcctgccattttagcaagtggtatggatggtgagtctccctgtgtaggttacgatgaccaatcatatggccttccacttcaagaatatttcactcacattgtcttatcaaataagccaagtgactttggtgctccatctaacgtggcgccaaataaatactacaacaaaaaatcatttgaacaattagctgacgaagataagacttatttaaatagtttatcatatcccctgtcctcatctaattattgttactttaatagcttcaaaacccaatcaagcacaacaatgctcgacaacgctgaagttattaaatatgtagttttgcttctcatgatcttggattatttcttttcaatcatttgcgaagatgctttaaacccagcatcttacgctatgttagttatcgttttggttcaggcttttattacaaaaattacagttttcagaacaggtatctatatccaggcagccgtttttcaagcatttattgtacctatagtcagtcaaattacattgatactggctgcagatactgcaagaagttttttaacgttccacttttttgtacttgctgttttgacatatttcgttctttgccgtattgctgtagatttttggcgttccatgtttttgctatttttgacaagcgtctttgcaaccatcatatggactacaaagaatgacttcaatattttacatgaaactggcgtggttctaacacccacggcagaattagctcttatagtagcttttacttatataatttatgcttcatgtatgttaacacctgtaccactgtatactatttgtgttttcttttcatttttatcaaatgctccactctaccttgccgtcctctcattcggcattctagtttctttcaaaacaaaccaagattttggacgtctagtggataaagtgttttctttaaatatgctctatgaataccatgcttaccaaaactatgttattcaaaactcaggtcaacacccaggattttacaggtcactctttgctttttttatcaatttgaccacccaaccaaaaacaacatacaaatgtttcaaaccccagacagcaagtggttacagagtaatatatcaaactcccactacagagttcaataaatctctgcaacatgccagtatcacaaaagatgacaactccaaccatataattatgtttgctgacggctcatctgataatctcaattgggcaaaagaaatggtcgcaaccattcatctaaccaacccaaatttgcagccactcatcattggatactaccacaactccatggacgtcataaccaagggaacttacatgcaacatgaattcataaaaatgccagctgttatcttaactcaagatcctctaactgaaccaatcagtcatttagcagcagcagcatttacttcaatttctggaaaacctcaggcacagaaaaacaacgttgtttcaaactccaaagcgcgcataaacacagccgttcacgacgctgtcgaaagcgtttattcaggagaaacatacgttgcccccaaacctatagtctcaggaaaaactgttgtagagaaaccattctctacaaccgaaaccaccatgtacataatgcgtggtttacccggttctggaaaatctttcaaagttagtcaattagttgctaaagatccaaatttagtcgtagcttccgcagaccactttagatattcaaatgacaaaactggaaaagccgtatacacctacattccagaagcaactagttctgtacatttacaatgtcagaatagagcccgcaaagctctagaaaacggccaatctgtgtgcattgataatacaaatctaacactcttagaaatgagaccttacgtcttattagcccgttcttttaactataacattgaattcatacactcagactctccctgggccttaaaccttgacctgttacatgctaaaggtgtacataatgttcctagagcaaagctcgtaatcatgtatgatagattctttgaccgtgataatcaaatcgatgcagacagtcttatacagtatgttattgaagcaattgatccaaaacttgttgctccaatcatgaaccgtttccctgccgactgtgatcttatccttcaatctgccctaacaccagaccttgaagtattaaagcaaaactacgacagagcaaacgcaacataccaagatgtttctttagatgatcctccggctttaaaggcagcacgtcgtgctatgaatatagctaaatctgaatatgaggcaggcgaagcaggccagcgtcgcattgagaaatttttagaaagacaggatgtagcagcactcaaccaaacgctcacaactgtcaatcaatctaaattcatagcagcgatccgttccatctacctaagcaccattagcaatttgagactaaaaacccgtcatatgggtgaaggatcatatgcagttacatcaggtactaatactaccgataaagttttagttaacacgccacaacgtatgactagaattgaagatggcatttataagcttgttgcaaacggttttgaaatcacaatgtgcgacggcagcaacttagccggtgttacttttgaacaggatataaatcctagcatgtacccttttgtttttacattaatgtcaaatatagctgtacctgttttaacccgccaagcaaatgttggctatcttgatatgtcaaataaattcatctgtaaagatggcactgttcaatttcaaggtgtcatctatgcctaccacactccatcaaatgagagtgctgacttcaaagtaggcaataccagttggaccctccagaaaaacatcaatttgactgcttttattcctgcaattcataaaactgcaaccttcgcagcacaatcagtgttcttaggaggactacccatggaagagcaccaagccttttccgacacacccacagcctcaaacaaatttaaagtttttgtttcatccacagtctgcgcctcaaccgtgtgcaaagtaaatcataaaacttatgtacagataccagatgacattcaagatccttttacatatatgcatcacagcgtttgttcacacaacaaatttttatcaaaccatgaaaccagatgtcaaatctgtcctttaaactgttanagcgcaaatccgtgtgtgtctacggcttgcgctctatttgataatggcacattacctcggtcaacacattatattaatgttagcaccacttcaaatgttggcttgtttaaggcagtaaagaagtctactcgtcaactaaacattgacggttttccttacatgctaaagcaggttaaagacgactcagaacttgtaagttctcttaaaataggtctacctaatatcctcccacatcacatggtggaaactaagtcaaaaacataccttcttaggggccccacaacggcttactcacttggcgatttatgctacgcactctttaatggcgactttgattatattcgcgaaaatataaactctgatttcgttttggaccgtgaagccggaatgcctgatacagaaacacgtacgtggctgttcagcattttaaactttgcagtacctagagtgtgtgctataattgaccagatgatttctgagaacgtcttctataaactgactttggataacttagatctatacggatcactctatgattttgacgactatcctactgaaggctttaacaggcctgatgatgtgatacgtatgttaaaggagatatggtccttctgtagacgtccactacctgccgaccttcttaaataccatgaagacatcggtgcagcagccactcaagaaatattgctgcatgcacccttcattgataaagtttgtgctctaaatgacagattagctgttgttgataatagagcaagtcaatactttttctgtgaagaagaaggtgtctttacccatatttacaatccagtctacggaactttagcattcgataacaagttgatccaatcaaaggatccttcatgtacattacagcgcctcattactatacaaggccctttgtctacgaatgctagtcccgtgatctctatttctgattccactcatattgccaacaatattaatccatctaaccaaaagacaacaccgttgtactacgatttggaacttgcgcaagaattcattgacgcaggtttaaatattgatggcgtttccaactacttcttctatggaccgtctagagcgggtgtagtgtctgatttcttactatatgaattccaaggaactcaatggtttgacaataacatgctgcgctctctttattctttcatattgaagaattcagagtgttacagaacaacagatcaactggactttagaggtggaaaaccccgtaaatcctcaatgggacatggtgttactggctttaagcaagacgtcgtgtacgctgctttaggccctgatatgattgaaaccttgtatgaaacggcaaaacaaacaccattgccgttttgtacaaaaataactgccaagtatgcattaacagcaaagcctagagctcgtacagttgcagcatgctcctttgtagcctcaactatttttaggtacgctcacaagcctctaactaataatatggtctcaaaagcacagcagggtttgggttattgtttaattggaatttctaaattccacggtcgatttaataaatttgttaagtctagggtaggcactgtcgaagactttaatgttttcggtagtgactacactaaatgtgaccgtacatttcccttagctttgcgtgctctttcagctgcccttattttcgatcttggcggccatgacccagacaactgtctttttattaacgagcttaatgcatacatgctagacattgtttcagtcgaagactcctttgcaaataaaccaggaggtacttcatcaggagatgccactacagcatactccaacactctgtataactttgcagtccactatattatcatgtggaaaacattcttgacagtcaatgacccttctaccaaggtcatacgcagtgcagctcatcacgccctaacaagtggtgacttctctatgtacaatgacatgatacaagacatgttggatgtagactatacactcaacttcctctctgacgattcatacatctgttcaaaaccaagcgcttttccgatctttacgctcgagaactatccttctaaactgcagtctatactccacacagcagtagatagcaaaaaatcctgggaagcaaagggtgagattaaagaattctgttcctctcacatagtcaacgttgacggcgactaccactttaaaccagaaaaggatagaatattggcttcattgctgatattatcgaaaatcgctgacatggacatcttctttatgaggttcgttgcgttattggctgaatccgccgtatatatacgcatcgatcctacattttggctggccctgtttggtgttttcgaaaaccgcgtaacagcgtttaaatctgaaacattgctctcacctgttcctgaacaactcatgaaggtggctttttatgaatcgcttgtctttgccgacgtggatgctacagccttatatggtttccttgatggttttaaaatgcaaagtcaaactctccacccagacggtgttgagggttttgacaagcaaagtgaccgagtaaaacactgttttgcttgtgacaatatatcagttggacactgttcgatttgtcccgttccccttcctttgtgctctttttgcttctatgagcatgctctgctcaatgaacattatgaagcttctggaattgcgtgtgaatgcggagacgctgacattagacaacttcacttaaaaataaccaatcaaccatcctcgcacaattttatctgtgctgaatgtcccactgtagctatgaagctgccaatcttcaactctttccaaggaaaagtactgcttccaatgttccgtatgaatacgccattgccttcctcagtctctgtaattgttgatgtacgttccaacccaaaagcacctaagatgctgtgggacgacgtccagaatttcagagaaaattgtactaggatagcatacgaatccgtttcgtgtgctgaactagctagggaggtggtttactatccatatgaagtgattgaatccaaagcaggtcaagcacgacttagaatacagaactttaaatgttcaccaactacttatgttcagttctacaaagtccgtcaaaatggaaagtattgtctagtagccaaagcaactctaacgccggcttttgaaaaccaaacagacattttctccgtttttcaaccaaacaacttttcaccttggaatacatcatcagtgtttgcagtagaacaatacgctgcaatataccctcccataccaaaggaaccagtcaatgctacgttcgtcttaggacctccaggctgtggtaaaacatactacatagccaaaacgtacttttcacaggcttctgagacatgtccggtcgtatactgcgcacctactcacagattagttttagatatggacgcagaatatagtggtgtagtttcaaaatctctctacaataatagagtgtacaaaaatccagcctacaaaacaggcgaaccattcaaattatgtttcaccacacacaacacgatgccagttcaaaagaaagcgatcctcattatagatgaagtgtctttaattacaccccactctctattttcgatcattggtaaagggttctatgagatagtactcgtaggagacccttttcagctctcggctgtttttccaggttttgttgtcaatcacacatatgacgggttttacatccgccggctagtaaataaggtcaaacacctaacagtttgttaccgttgtccacaagaaatcttggacatattttctaagccctatcatgatgttgggattgacctcacaaccggaaataccaatccaggaaaggcatccatttatacactaaattggcttcaagcagatgtaggtactaaaaatccggacaaactcagacaactctttgcgcaatatccaggctttaaaattatcaccaactacagatgtgttgttgatgcagctaaaagttacggtattaacgtcgaaaccatcgactcatcccaaggaaccaccggagataggcatctggtggtaatttgcggcagtaccaacttttctaaacttttaaacaggtttatagtagcagcctctcgttcaacaactgaactagttatagtcatgttgccagagctttacaactatttaacagagacgtttaacttcaaaccgttacaattgcaaaatgtgcatgtaccgatcgcagtatcttctacagcattctgcgatatagaattttatcactttcaaaagaagttctatgttggtgaaataagcgtaagcacaagtaccactatgacatgtcagttgggttgttatattaatggctcctacatgctcccacctgtgcttgaaaactctgaagaccgtctctacgttccttctagatggagacgtatgataagaaaataccctactgaatctatgcacatttccttactggacagacttctgaggcacattttattaacaactactggagaaattcatttcgtaatgttctctgcagacaatgatctcattgcactggatccgtactttataccacccactctatgtgagtgtggtagtgcgggtctagtggaagtagacatcactgttttctgccgcaattgtttgcctaaagatggtaaagccactcgtttggtaaaaccgtctacactagatgtccagactgaaaaactcagacttgcaaaagttcatgctaaggtttgtaaaatcaagcatggcagtgctcacaacgctgatgttgatgctattatgactcaatgtatatatgctaatagcttaacattcacaccaacaacccaactagttgttaacactgatgagttcaccttttacatgctacctaggccgtcaaaccgtcatttgagaatcattcataagaacgacaaacgtttctatgctatcactcatgaagaagaagatctcttctttactaacatctcagcagtggtagacccaattcctgcaaaattcaacattgcacactctacaagcttcctcaccatcaaaagtggttgcgcaggtaataagacttgtaccagatgctattatttacacttagcatacacggaatttgtttctcaacacaagtatgaaccattcacttgtgtgtcttttaagatacggtttgacttttcacaattcactgactcagtagatactttcctccgacaaggcttaataacctttcatccggagatgaattcactgcaaaaatcacttttattagcagtggataaggtctattgtgataacttcacctcaaacggtagaaggtttagactttacgacaacaatttggttaaatccataatcaaaggttcagtggctcaaaactccatcatcatgccactcgactcagttttacacgggttgaacattgatttcacagtcggatgtgccgtagataacttttcctgcaaagaagcagcgagtgttaggtactcagaagtagtactttccatcaccaagttgcccccaggcacttgccagttatactacgtcatatcttacggcctgaactctcccaagaccacttatgctggtcaccaattgttcgacggctttgagactgttattgttgttaatcgtaaagataaacccccttacgtcctcacacagtatattaatgatgttgtagttgcaatgccagagtccctcttttcaacaggtcgattctacagagaaaaaccatatcccgtccttatgaacgaggatttaagtggcttaaaccatcacattttctctggtgactatacagacgaatctcttacattaggaggtgtccatcatatagtaactttaaacacctatgaccacaagctcaactatatccaaacgaaagctacatgtgccgcctcagtttcaactggcggacgtggtcataaaattactacactgtttgacgttcatgcaaatcaacttgctgatgaaattaccagagttacatctgttgttacaacacagtctaaagttattaatttgacaatagattatcagcaagttccttgtatgtactggtcttcaccgaccggcataagaaccttctaccctcaggctgttagactggacgcaaagtttataccatactacgtagaatatcccaatattctaccggcagttgttgaagaccaggtgtacgatttgtctaattacaatcaaccacctttaggccaaaactgccctgtaaactttcacaagtacgtccagctaactcactttattttagatcatgtgaaaatccccgaaaagggtttgatatatcatatcggtgcagcaggtactaagcaatgttcacctggagacttaatattggaacaatttttcaataaatccatcatatactcaagtgaccttcttccttaccaatcacctgctgtgcaggttgcattggatgtaaggttttcggcttcactcatcatttcagactgctattcgaaagaaccgcagcctgatttgttgagtaagttgattaacaaactagtgtatggtggaactctcatttttaagaccaccgagactttcacatgtgacccagccttttatgttgctcattttaactgtataaagttttttactgccgctgttaatcactcatcatcagaagtttatattgcgttcatcggaaaactccctaaaccaaacaacaactttttagcctcagactatttccagagattaactcaacatagaaataaagtagttaaacagccttacgctcacacatgggacacatcttttacgtacccatacccctcaaatgttcttcaagttagtcgtaaaaaccttttatatctatttgaaaccagaggagctgcagtaggtactttgatttttgaagaaccatcaaaacctgctgtaaagatacctacaaagtgtcaaaccacacaaccctcgtgtgtcattgaggttggtaaccaatacgattgttgcattcaagacatcattaccctcctcaatggaaaatccttcacagtgaaggtgcccaactcagaatccttactgcgcgatatctgcacacttgcgcttagccagagttattccatcaatattcgcggaaaaacactctacacccttagttccctacttagaattaggcaacaatccttactgttttacggagagaaggtcaaaaaccctcgaccccgtaatgtcttgaacaaatataccaactacctcaaggcaaaagtgattaggcattacaccaagcctcaatcaacagttttggacattggtacaggaaaaggacaagatttgagaaaatactcgttagcaggggttaaatccctcacttgtgtcgagcctagtcccgagtctgtgactgaactttcaataatagctagtccccttgatatggagacacacacagttatgagttctgcccagaaattcgagacctcgctgacgtttgacttggctttctctttctttgccttgcactatgcattggatgacgtttgtatgtctgaaacactcaacaatgttttttgcaaacttaacagtaattcacagttgatcttagtagttccaaatgctggcaggatgcaatccataccttcccttggtttaacagtcactcatctagatgatgataaagtttggtttaaatactcagactatatagactgcgaagaaccgttagtagacaaagaaaaactacttacgtgtttagctacatatggaacaattgttactgactcaccattctatgacggtgcaaacaaaatcctagaccaaaaatgctcatccatgtatagagcatcgacagcccatctaaatcccgatgaaattcaatatattaatatgtatgatttaattgttgtcattaagaattagcacaacaaaatgttcgcgctcgttctaaccctcacaatagcttcggctattgcccaagatttccccgcatatgacccgtgtcctacttgctcaacccccggtaataaaataccggctccgagcacagttgcccagtattcaacaaactacggtgcgaacttctttaccgtagtctttgatggtattatcttcaaccaatttagggagagttattaccaccaatgtagaccaacacctgaatactgcccagatgcaatcaattgcgccttaaacagaacaggcgcatcctgcaaacctttcgcaactggcccgaattcacaatgtcagaacagtttcgagggcaacatcgacatatgtgcaacatgtagccctctaaaacaagaaactccattcatctgctacaatagatacgggataattatatacccgacagcagatatcgttctctccgctaggtttaagataggctctttttcacccaaggcttgtgataactacctaaacgacttaaattgtgattcaaaaacggcaaggtcatatgtcatttcccgaccgcagtctttttcactgcaatatcctaactcattaggcccctatcagctgaaacgattttctcttgcaaaggagatcgttgacttacgtgctggcgttttaacctcactcccaaaccgaggttataagggtagaacaacatactcttatcccgtcactgcactctcactcttggctcgttccaaagtggctgaagccgacaaattcttttatatcgaggctaaaattctactgtacgcttggtcacagaaacctcaaatccgctttctaggtgcatactgtcccacagacgtgtcatgccctgattcaactgccctcggctgttgtttttccggaagtggatctgagttttactacgcctttcgccagtggtactacgcaagcctgggtatggaagacctagtcgactttgataattcaacagtcttaagtctctcgcctgatactcctcaaattacacccgttgtgtcttattttctagaaaaagttttacctttgtttaaatcacatgtacccggacgtgttttttactgccattcacttatgtctaacggtgtatgtacttttgaccatgttgttgtaaatattaatgccgaggccgtcttttttgacctcgaagtagacataggcagcataattgctgacgcatatcgcgttgaaaggcctaatactttatgttatgatacaaactgtactcttgccacaagcaggaccactgagtataattacgctgcttatgttgtttatatactcttcaatttgtattctagtaatcgcattgcgatagatttcaacacacactcaatcttgcaaggattactacaacacaatagcaactaccagactgctaatttagactatctgtttgttggagcactttttacaggtacttttaaacatattacaagcaatcaagcttacccagtacctttaacttatccaattgttaagacatatgtagggccgtcaaaccaatactcaatgtcaaataaactgttttcatatactcacaatttgacggctcaagcccattcaggcatatgtaactctttttactgttataaaccacgttttgtaccaattgatgtttttattcatagtgctttaacccctgacagcttgatggaaacagaatcttttgtttgtgtctctttgcgttcaccatctgcaggatcaacatccgcaggtagtttttatttgcaatgtctcaattcttccatcgatttgcatccaggttcatttgtacccgtttcctcaagtccagagtcttccagccgcgtaacagctgagctggcttttaatactagaaatggtatattttctccttgtcttaacggtacatgtgtactcgcacctactgacccaattgtttttatgcgtcagggtgcctggtttacaaaatctttacactttgatgtttcaccatgcaaacctatgcattttccagacatagatatacagcccccaacatacaatgtctcctctatcaagatggacgacaatgctgtattggttcaagaccttacttcgggtttagtaattgaccacaatttaggctccatactcagaccgaaaggtagagctttggaagtttcgtattatgctcactccattttacgttaccttgaaccggattcttgtctacctgacaactttcttaactttgtcacttgtttagactatatctgttcagactcgtcaccttgccgtgctgccgcaagccagtactgtcaggcaggcatttattttgagtctgcatttaataagtctaggtattctttgcttaacgcttacacgctttttaacacaagtcttcaaaccttattgcctgagacttttcttgagatagaagatgatgaaccccatagcagatcaaagagatcaattgatactacaagcaatattcgccctagtcaattgcttgttaatggacgtattccgtctacaagttcagcttttgctgttaacgtcgctcgtggtcgaggaacgattatgcctcgtcctggaactggtggcatgggttcgtccttttctgctgtttctaggtcgggtagtatttcttccttatcctcggttggctcctcaacacctttgatctctaattggagaacatcttcatctcaactcaaaactctcaacctcaacattaacactaaaattcctaagatttcaacaaagtcaggttttgccagtattacatctttgtttgcttcaggtttaggagtcgtcgatctaggtctatctattttcaacatgatagaacagcgtagagttgctgagatcactcagatgcaaattagccaactggctgactctatagtgtatcttgctgatgtgacatttgaagctatcaagaatttggaactctcggttaactccttgggtacgttcttatcggaattttccactcagatgtcgatcaccataagccaaatacaatcatcatttgaagagcagcaagatgctacaaatgatgcgttgtactacactaacgctgctgcgtcataccaagcctccatggcgtatgtcatttcagagttaaacgcaatatctctgtctgtcactagatcctacgactcttacaccagttgcatcacttctggcattaatgggctcattacaccatcatgcttgccagcccaccagttgttacagttactcgacaccgttatcaattccacagcaggaacaggatgccgtcccatctacggcagagaagaagtggtgaaatactacactttacctctaatcaatcaaggttattcctttaacgggtcgattttcttcgtctttaacattcccatcacttgccagggaattgccggagatgtatatgaagtagaaccacctatacttgtagatgtaccatcaaagactgctttacgcatgattacaccatcaaacgtagtcgcaacacaagcaggattagctgaattagatttgcgtcattgcgaaaggtaccataacgagttcctatgcgattcttcagcattcctttctacaccttcaaaatacatagactgtttaacaaacgcaactgactgttctttgcaattcatcacacaacacgttccagatccttgcgtttacacatcgccagcttctttatattgttattattcacccatatgtgatcaatgtcacatagtagccggttgtaatgaatctcagcagtacaacttcacttctgctgatggcggcgtagtcttttattccatacaagacagagactgtggccacttcccccacatcactgttactacgcctgcagccatacaagaagacttcactgtcggaccgtatttaccatcgctgccaattcacaccgcctacgtcaatgttacctggaatgtaacactaccaggaaattggacctgggaaaatatcaccctaacagccaattggacccaacacttcattgagatgaaaaaaaacatcacaatgatggctgaagaaatagataaccttaccaacttcggtaaggttttagttggccagctaaatagctttttatcatctttgtttaacataccattaggtttgatgacgttttgcttttctgtagccgctttaggcctgtccattattgctttacttgtgttatgttttccacagaagccacataaattataatcgtggtttcgcttgtaaatattgatcaattgaggttttttacactttagtgtttttcctcaaccaattagaccagaggtttttttacaccaaagtgtttttcctctacaaagaattgaggttttttacactctagtgtttttcctcaaacttatatatataaaatttcattagtttgacatttcattataaatagcacaacaaatacattcaggcgacttgcatgatgtttaccctagtagtgctttttaccctcctcggcctttccatggcctccacagagctgaatttcgatcctactctacccctcccctctcctataaatgccctcgtcgacattttcggaaacaacagcttgtttctcaaagagtccctgctcggcaaatccaccggagccgtctacgcatacttgtacagcagtgccatctctctcctgctgctactttgggtaactgtatggagtattgctacttcacactttaacgtaactcgcattccaaccatcgcggttctcactaatgcgagtatgtttttgctgttggcatcggctactgttacaacctggtttctcccaactgtgacgaacgtcttcttttatacactcactgcgctgttcaccttcttttcctttgtgttcttactgtggttggtttactatatgtttactaccattagggcatatcgaagggtcggttcatggcgcgttgtgtttaacggaaaatattctctacttgctggaactcaggctgtttgcctttgcagacccgccatacatctggttctaaccaaaacgaacacagatacatactggtgtctagatggaacccccatctacaatgttgacttactacaattagttggccccaaaggattatatccttacaaaagaatgactacaatcactgcaccaaaaggcacaaaaacatctgctgccgtttacacccttcaaaaagaagaagtttgtgctctctcagaaatcacagtacataatgatactgatttttaggtcatataaaaaagctaacacatctaaaaaatgtcttacccggtttactacgaacagcgtcgttattccccccgccaattcaacaatggcggagggtataatcctacacctcaacctagagtagttcgtactaatcctggtaaccaagcttacaacccccggcgtaaccgaaacgccactccgaaccaacaacaaatggttccttaccagcctcagtatcaagcacctcctcagccaagggtggtctatgtagatcgccctcaagaacctgtagtaatttacagagctcctccacaaggaaaaaaacaatcaggcaaacgccacacagcagaagaacgctggtatcaaggcgaaaaacctgtgcagaagaaacaggcacccaaaggaaaatcaaagaaagcagcaacacctgctaatcctaaaaagcagcctacacaatctgacaaagttcccatcgcctacccagacaatcatcccttccatgacctcgcaccagctgacatccgcgctttcaaaaagcagctgatccaaaatctggaccttggacatggtgaaatgaatcaactgcggctttcaatcgatctgttgcccatcaagaaaccagcaccaacaccagcggtgccagctcctctgtaatttatggaaaaggtgcaagacctcccataaataagtagatatgttcattaccctcattatgatcttcgccatcttggctttcccttcaacatctgaaggagcagcccaagaactactcaaagctgtaaaatctgctgctatcatggaaaaggtgcaagacctcccatgaaaatagtagccggtttcaaaagttgataattattgcattatgtttacccttgtgcttattatcctgcttagtttttctatggcttttaatgcttttacatttctgctgttattattttttacttttaagtgcattataacccgcactttagtcgtagttcccattgactacccagaaaatcatcctttcaatggcctctcaccagaggaaatcatcagctacaaatcacagctgatccaaaatctcgatcttggacatggtgaagtaattaaacatcgattctcaattgatttacttcccctcaaaacaacaagcactcctaccaccagtgctattttatggaaaaggttcaaaacctcccataaagaaaacaaccactaaacaaccatggaaaaggtgcaagacctcccatgaaattagtggttgctttcaaaaattaataaatattgtgataaatgtctatttctaaccacccttaaaaataggtacccccactatatctagccgacgttaactcctggatatgttatagtgttctctccccaatcgttcatctgctcgcttttagaactgctaggctgtatctgttaaatgtttaatctttagactcaatttacgttcttttttacataaaatcctcctattttgctatcccttattttaattaaacccctttagtatcaccagtatccctaatcactcccctagcctcccttgttctgacctgtatgaaatgtcaaaaaactaaatgaaaa

SEQ ID NO: 7 - protein, 5867 aa, unknown
  Fish coronavirus ORF-1 amino acid sequence
  MISC_FEATURE (830)..(830): Xaa is any amino acid
  MISC_FEATURE (831)..(831): Xaa is any amino acid
  MISC_FEATURE (832)..(832): Xaa is any amino acid
  MISC_FEATURE (833)..(833): Xaa is any amino acid
  MISC_FEATURE (834)..(834): Xaa is any amino acid
  MISC_FEATURE (835)..(835): Xaa is any amino acid
  MISC_FEATURE (841)..(841): Xaa is any amino acid
  MISC_FEATURE (842)..(842): Xaa is any amino acid
  MISC_FEATURE (3257)..(3257): Xaa is any amino acid
MKKIEDALGTLKPAFKAHIDSLPPFMGKLATMLAETRRGKTPPLLIYVISTILETNITVHYVSHTITSYHSSNAHTHTHEFDSEDYTPDNQILTKVNRHGNDLNHSYIHGATNMYNPVYQRHPPNMCYMLTGLYMLSGLQELYAMAEDNLTTCQINLLRCLFDLNQDEFDVDYTFVIYTPSKSQECAFKYLQEIVHHCELTIFRHTTTSVFSCNKCNHVETVISSCSLNLVYITDSIEKAFQPTVEANTDYMCENCGLRDHKLKTTVTNPDLRLAQLNYPTDSKYTIFLDEQAPFVFHSIAKHVGTANSGHWSALNVNSDMLSDSNERQHYYTTPSIVLLAFLPEEELQNIRNSSPLQDHQPDDVEDVESPLPGSIFYTTDDIFSTKSLSIAHCVARDFHMSGGIAKIFSDKFGSKTFLKSQNPVIGGFSILLRECRDMYYLVTKEKTSDKPTYQDLKNSLGSMTENLVRKNHNTLSIPYIGCGIDGLQWATVEKQVKEIVCARGIDVTVHHLENEVKHTPEQQTASDNSVKLVQKLFTETPQAIPIVVPSDDSDSDIDESADVFLPGPESESDSKSESGSDYDFKSASEPEDELEPTPISELELTPASSLTVESDDNPDTSQETLPESNSEETKPEQTPDTTSKVSSDSKLDPQSELEEELANKPESASEPQSETESSSESEEELEPQSESEEEPANKPESPSESQSENGSSSEPEEESEKPSESAETATEDSPETTPETTLELTTQLKPASESDDKPDTPAPSPSPIQPEKNLDTTPEQTSQPTTQLELTLETQEQPDTTPEVPSVSEDKPDTLEESSESTPELSELXXXXXXTSTKTXXTRPPTPRPRSARGARTRSCAGTPIPVIFDIIDNTSQPQVPLDFPEALQELNKPSEVIPAASEKPVEKQIIHSFVSVETPCKPKATKVTNYVAAQSNAILNCIKAFIPSNPLSLFNRKPAFRKIIFTEDTSEPDSDDDDCEYTPPTSPFPELLALVDEDIEVEQTQSVIPKTDSASIVEDLKKQESSTLSLDTNTSKPTSSPRRQPREVESVDESSDDSSKPKTISTLDKPAMNSDTKPTDSSRKEPLEVPVTTSLSTPAKNQDKKSSKSAKVIKDYSLTPNTVKQQVYSLYGESVDAVKYLVQTYPDRAKQTAGIAYFLITTYLIWTIGLIGVPMAFKIPMFLCLLYQVNGLNIAPFVTNQKLQYVAFPLWYKLYEVISVRFVANIAQFIVKTPPIDVLNKLIRSNKDKPVKLTPNKHTLMLIHDLALESVDGKENRYYNTDVTTFTKRHSTSNISYVLKSTLIKYVMDHCYVNIAVFTLVRYLTLLVFIQHFSNPYVLEANSQSHTVLQYLFSHLRPFGRPLCPTLNDYMTTATPRDAHVQAGSHFSEFCVPIHYTTPIIKSTMAEPSLFLLFNPVLWPLVMVVYFYPPMMFIANAVAYSCLPLVVLLQWLYAMWFSCTCYGTKRCAKHLHKNEVVKPMESTSTKNRMTFTPSTTFCSKHNFFCPDAPHIMTLAMARQLTNYYNLTDTVIPDIQEYSHENPTVQFIHFDPLKHGADTILEPITSASASSIVAWYSLLFNQKFVLSHYSYRTPVAVVDKPEETDGDDTKSLASDTSDNFESIRKTNHKNQSKQQFRPNGHQRPSKTFKRHSRIMTSEQKNSLIETFKGLTNGTAAIPQPLIIFIWVILMVIPTLFLVASSSRTAATMPLNRYSGVNPTGIMFHQAPPYIHSEPPKETYYKLSYPYPSATVVRTLKGHLYYHSDDTVQQNCTMQYSLIAASTKHVCGKVVYTIPAHVSIGSLKLLLVHPDQTNLPFELPVSDEVRLCYLTTLNAPRCMPSQLAMSNNQFAAVSLVLLITLVSLIKVYIMFFTVFKHYTTTVFILVAVTTITMLVSFLAPPLLIVVLLSLAWLWYGNTIVLCHIMLLIVLVVSWKVAAVCFIFALLYFGKCAMLSKNIKYVQGGVKFSGTFEEIAQSTFFINYGVACQLLEHTGQTIEDIMQLRTAGGAPARLARSIYDCFSTNASVLYSPRSFSPQSLITKYLYPGSIPVGRAPVLLGKISGMTCLGREQSTCFQSSATTITTCTHAVNTAGTFMSQIKCVIDNKIYTVQPENITITGMKATFEVEGLPPFTNDVTVAPKPLKHYMDGKRHLVLYTKSESIVYSSIMWPTENGLFSSSVSDPGDSGAPYFSDNVIVGIHQGRNEATNNPAILASGMDGESPCVGYDDQSYGLPLQEYFTHIVLSNKPSDFGAPSNVAPNKYYNKKSFEQLADEDKTYLNSLSYPLSSSNYCYFNSFKTQSSTTMLDNAEVIKYVVLLLMILDYFFSIICEDALNPASYAMLVIVLVQAFITKITVFRTGIYIQAAVFQAFIVPIVSQITLILAADTARSFLTFHFFVLAVLTYFVLCRIAVDFWRSMFLLFLTSVFATIIWTTKNDFNILHETGVVLTPTAELALIVAFTYIIYASCMLTPVPLYTICVFFSFLSNAPLYLAVLSFGILVSFKTNQDFGRLVDKVFSLNMLYEYHAYQNYVIQNSGQHPGFYRSLFAFFINLTTQPKTTYKCFKPQTASGYRVIYQTPTTEFNKSLQHASITKDDNSNHIIMFADGSSDNLNWAKEMVATIHLTNPNLQPLIIGYYHNSMDVITKGTYMQHEFIKMPAVILTQDPLTEPISHLAAAAFTSISGKPQAQKNNVVSNSKARINTAVHDAVESVYSGETYVAPKPIVSGKTVVEKPFSTTETTMYIMRGLPGSGKSFKVSQLVAKDPNLVVASADHFRYSNDKTGKAVYTYIPEATSSVHLQCQNRARKALENGQSVCIDNTNLTLLEMRPYVLLARSFNYNIEFIHSDSPWALNLDLLHAKGVHNVPRAKLVIMYDRFFDRDNQIDADSLIQYVIEAIDPKLVAPIMNRFPADCDLILQSALTPDLEVLKQNYDRANATYQDVSLDDPPALKAARRAMNIAKSEYEAGEAGQRRIEKFLERQDVAALNQTLTTVNQSKFIAAIRSIYLSTISNLRLKTRHMGEGSYAVTSGTNTTDKVLVNTPQRMTRIEDGIYKLVANGFEITMCDGSNLAGVTFEQDINPSMYPFVFTLMSNIAVPVLTRQANVGYLDMSNKFICKDGTVQFQGVIYAYHTPSNESADFKVGNTSWTLQKNINLTAFIPAIHKTATFAAQSVFLGGLPMEEHQAFSDTPTASNKFKVFVSSTVCASTVCKVNHKTYVQIPDDIQDPFTYMHHSVCSHNKFLSNHETRCQICPLNCXSANPCVSTACALFDNGTLPRSTHYINVSTTSNVGLFKAVKKSTRQLNIDGFPYMLKQVKDDSELVSSLKIGLPNILPHHMVETKSKTYLLRGPTTAYSLGDLCYALFNGDFDYIRENINSDFVLDREAGMPDTETRTWLFSILNFAVPRVCAIIDQMISENVFYKLTLDNLDLYGSLYDFDDYPTEGFNRPDDVIRMLKEIWSFCRRPLPADLLKYHEDIGAAATQEILLHAPFIDKVCALNDRLAVVDNRASQYFFCEEEGVFTHIYNPVYGTLAFDNKLIQSKDPSCTLQRLITIQGPLSTNASPVISISDSTHIANNINPSNQKTTPLYYDLELAQEFIDAGLNIDGVSNYFFYGPSRAGVVSDFLLYEFQGTQWFDNNMLRSLYSFILKNSECYRTTDQLDFRGGKPRKSSMGHGVTGFKQDVVYAALGPDMIETLYETAKQTPLPFCTKITAKYALTAKPRARTVAACSFVASTIFRYAHKPLTNNMVSKAQQGLGYCLIGISKFHGRFNKFVKSRVGTVEDFNVFGSDYTKCDRTFPLALRALSAALIFDLGGHDPDNCLFINELNAYMLDIVSVEDSFANKPGGTSSGDATTAYSNTLYNFAVHYIIMWKTFLTVNDPSTKVIRSAAHHALTSGDFSMYNDMIQDMLDVDYTLNFLSDDSYICSKPSAFPIFTLENYPSKLQSILHTAVDSKKSWEAKGEIKEFCSSHIVNVDGDYHFKPEKDRILASLLILSKIADMDIFFMRFVALLAESAVYIRIDPTFWLALFGVFENRVTAFKSETLLSPVPEQLMKVAFYESLVFADVDATALYGFLDGFKMQSQTLHPDGVEGFDKQSDRVKHCFACDNISVGHCSICPVPLPLCSFCFYEHALLNEHYEASGIACECGDADIRQLHLKITNQPSSHNFICAECPTVAMKLPIFNSFQGKVLLPMFRMNTPLPSSVSVIVDVRSNPKAPKMLWDDVQNFRENCTRIAYESVSCAELAREVVYYPYEVIESKAGQARLRIQNFKCSPTTYVQFYKVRQNGKYCLVAKATLTPAFENQTDIFSVFQPNNFSPWNTSSVFAVEQYAAIYPPIPKEPVNATFVLGPPGCGKTYYIAKTYFSQASETCPVVYCAPTHRLVLDMDAEYSGVVSKSLYNNRVYKNPAYKTGEPFKLCFTTHNTMPVQKKAILIIDEVSLITPHSLFSIIGKGFYEIVLVGDPFQLSAVFPGFVVNHTYDGFYIRRLVNKVKHLTVCYRCPQEILDIFSKPYHDVGIDLTTGNTNPGKASIYTLNWLQADVGTKNPDKLRQLFAQYPGFKIITNYRCVVDAAKSYGINVETIDSSQGTTGDRHLVVICGSTNFSKLLNRFIVAASRSTTELVIVMLPELYNYLTETFNFKPLQLQNVHVPIAVSSTAFCDIEFYHFQKKFYVGEISVSTSTTMTCQLGCYINGSYMLPPVLENSEDRLYVPSRWRRMIRKYPTESMHISLLDRLLRHILLTTTGEIHFVMFSADNDLIALDPYFIPPTLCECGSAGLVEVDITVFCRNCLPKDGKATRLVKPSTLDVQTEKLRLAKVHAKVCKIKHGSAHNADVDAIMTQCIYANSLTFTPTTQLVVNTDEFTFYMLPRPSNRHLRIIHKNDKRFYAITHEEEDLFFTNISAVVDPIPAKFNIAHSTSFLTIKSGCAGNKTCTRCYYLHLAYTEFVSQHKYEPFTCVSFKIRFDFSQFTDSVDTFLRQGLITFHPEMNSLQKSLLLAVDKVYCDNFTSNGRRFRLYDNNLVKSIIKGSVAQNSIIMPLDSVLHGLNIDFTVGCAVDNFSCKEAASVRYSEVVLSITKLPPGTCQLYYVISYGLNSPKTTYAGHQLFDGFETVIVVNRKDKPPYVLTQYINDVVVAMPESLFSTGRFYREKPYPVLMNEDLSGLNHHIFSGDYTDESLTLGGVHHIVTLNTYDHKLNYIQTKATCAASVSTGGRGHKITTLFDVHANQLADEITRVTSVVTTQSKVINLTIDYQQVPCMYWSSPTGIRTFYPQAVRLDAKFIPYYVEYPNILPAVVEDQVYDLSNYNQPPLGQNCPVNFHKYVQLTHFILDHVKIPEKGLIYHIGAAGTKQCSPGDLILEQFFNKSIIYSSDLLPYQSPAVQVALDVRFSASLIISDCYSKEPQPDLLSKLINKLVYGGTLIFKTTETFTCDPAFYVAHFNCIKFFTAAVNHSSSEVYIAFIGKLPKPNNNFLASDYFQRLTQHRNKVVKQPYAHTWDTSFTYPYPSNVLQVSRKNLLYLFETRGAAVGTLIFEEPSKPAVKIPTKCQTTQPSCVIEVGNQYDCCIQDIITLLNGKSFTVKVPNSESLLRDICTLALSQSYSINIRGKTLYTLSSLLRIRQQSLLFYGEKVKNPRPRNVLNKYTNYLKAKVIRHYTKPQSTVLDIGTGKGQDLRKYSLAGVKSLTCVEPSPESVTELSIIASPLDMETHTVMSSAQKFETSLTFDLAFSFFALHYALDDVCMSETLNNVFCKLNSNSQLILVVPNAGRMQSIPSLGLTVTHLDDDKVWFKYSDYIDCEEPLVDKEKLLTCLATYGTIVTDSPFYDGANKILDQKCSSMYRASTAHLNPDEIQYINMYDLIVVIKN

SEQ ID NO: 8 - protein, 1391 aa, unknown
  Fish coronavirus ORF-2 amino acid sequence
MFALVLTLTIASAIAQDFPAYDPCPTCSTPGNKIPAPSTVAQYSTNYGANFFTVVFDGIIFNQFRESYYHQCRPTPEYCPDAINCALNRTGASCKPFATGPNSQCQNSFEGNIDICATCSPLKQETPFICYNRYGIIIYPTADIVLSARFKIGSFSPKACDNYLNDLNCDSKTARSYVISRPQSFSLQYPNSLGPYQLKRFSLAKEIVDLRAGVLTSLPNRGYKGRTTYSYPVTALSLLARSKVAEADKFFYIEAKILLYAWSQKPQIRFLGAYCPTDVSCPDSTALGCCFSGSGSEFYYAFRQWYYASLGMEDLVDFDNSTVLSLSPDTPQITPVVSYFLEKVLPLFKSHVPGRVFYCHSLMSNGVCTFDHVVVNINAEAVFFDLEVDIGSIIADAYRVERPNTLCYDTNCTLATSRTTEYNYAAYVVYILFNLYSSNRIAIDFNTHSILQGLLQHNSNYQTANLDYLFVGALFTGTFKHITSNQAYPVPLTYPIVKTYVGPSNQYSMSNKLFSYTHNLTAQAHSGICNSFYCYKPRFVPIDVFIHSALTPDSLMETESFVCVSLRSPSAGSTSAGSFYLQCLNSSIDLHPGSFVPVSSSPESSSRVTAELAFNTRNGIFSPCLNGTCVLAPTDPIVFMRQGAWFTKSLHFDVSPCKPMHFPDIDIQPPTYNVSSIKMDDNAVLVQDLTSGLVIDHNLGSILRPKGRALEVSYYAHSILRYLEPDSCLPDNFLNFVTCLDYICSDSSPCRAAASQYCQAGIYFESAFNKSRYSLLNAYTLFNTSLQTLLPETFLEIEDDEPHSRSKRSIDTTSNIRPSQLLVNGRIPSTSSAFAVNVARGRGTIMPRPGTGGMGSSFSAVSRSGSISSLSSVGSSTPLISNWRTSSSQLKTLNLNINTKIPKISTKSGFASITSLFASGLGVVDLGLSIFNMIEQRRVAEITQMQISQLADSIVYLADVTFEAIKNLELSVNSLGTFLSEFSTQMSITISQIQSSFEEQQDATNDALYYTNAAASYQASMAYVISELNAISLSVTRSYDSYTSCITSGINGLITPSCLPAHQLLQLLDTVINSTAGTGCRPIYGREEVVKYYTLPLINQGYSFNGSIFFVFNIPITCQGIAGDVYEVEPPILVDVPSKTALRMITPSNVVATQAGLAELDLRHCERYHNEFLCDSSAFLSTPSKYIDCLTNATDCSLQFITQHVPDPCVYTSPASLYCYYSPICDQCHIVAGCNESQQYNFTSADGGVVFYSIQDRDCGHFPHITVTTPAAIQEDFTVGPYLPSLPIHTAYVNVTWNVTLPGNWTWENITLTANWTQHFIEMKKNITMMAEEIDNLTNFGKVLVGQLNSFLSSLFNIPLGLMTFCFSVAALGLSIIALLVLCFPQKPHKL

SEQ ID NO: 9 - protein, 260 aa, unknown
  Fish coronavirus ORF-3 amino acid sequence
MMFTLVVLFTLLGLSMASTELNFDPTLPLPSPINALVDIFGNNSLFLKESLLGKSTGAVYAYLYSSAISLLLLLWVTVWSIATSHFNVTRIPTIAVLTNASMFLLLASATVTTWFLPTVTNVFFYTLTALFTFFSFVFLLWLVYYMFTTIRAYRRVGSWRVVFNGKYSLLAGTQAVCLCRPAIHLVLTKTNTDTYWCLDGTPIYNVDLLQLVGPKGLYPYKRMTTITAPKGTKTSAAVYTLQKEEVCALSEITVHNDTDF

SEQ ID NO: 10 - protein, 201 aa, unknown
  Fish coronavirus ORF-4 amino acid sequence
MSYPVYYEQRRYSPRQFNNGGGYNPTPQPRVVRTNPGNQAYNPRRNRNATPNQQQMVPYQPQYQAPPQPRVVYVDRPQEPVVIYRAPPQGKKQSGKRHTAEERWYQGEKPVQKKQAPKGKSKKAATPANPKKQPTQSDKVPIAYPDNHPFHDLAPADIRAFKKQLIQNLDLGHGEMNQLRLSIDLLPIKKPAPTPAVPAPL

SEQ ID NO: 11 - protein, 113 aa, unknown
  Fish coronavirus ORF-5 amino acid sequence
MFTLVLIILLSFSMAFNAFTFLLLLFFTFKCIITRTLVVVPIDYPENHPFNGLSPEEIISYKSQLIQNLDLGHGEVIKHRFSIDLLPLKTTSTPTTSAILWKRFKTSHKENNH

SEQ ID NO: 12 - DNA, 18 bp, Artificial Sequence
  TCL-ORF-1 Forward primer
acactataccagcccatg

SEQ ID NO: 13 - DNA, 22 bp, Artificial Sequence
  TCL-ORF-1 Taqman Probe
caagcaacagtttaagtgagcc

SEQ ID NO: 14 - DNA, 18 bp, Artificial Sequence
  TCL-ORF-1 Reverse primer
tgtgaggtagcaaagacg

SEQ ID NO: 15 - DNA, 19 bp, Artificial Sequence
  TCL-M Forward primer
gtgttcttactgtggttgg

SEQ ID NO: 16 - DNA, 22 bp, Artificial Sequence
  TCL-M Taqman Probe
cattagggcatatcgaagggtc

SEQ ID NO: 17 - DNA, 19 bp, Artificial Sequence
  TCL-M Reverse primer
tgagttccagcaagtagag

SEQ ID NO: 18 - DNA, 20 bp, Artificial Sequence
  TCL-S Forward primer
tcaaaactctcaacctcaac

SEQ ID NO: 19 - DNA, 24 bp, Artificial Sequence
  TCL-S Taqman Probe
aagatgtaatactggcaaaacctg

SEQ ID NO: 20 - DNA, 19 bp, Artificial Sequence
  TCL-S Reverse primer
acgactcctaaacctgaag

SEQ ID NO: 21 - DNA, 18 bp, Artificial Sequence
  Primer
taatttgactgactatag

SEQ ID NO: 22 - DNA, 25 bp, Artificial Sequence
  Primer
taagaaactataccagtccatgtcg

SEQ ID NO: 23 - DNA, 12 bp, Artificial Sequence
  Primer
agtttaagtgag

SEQ ID NO: 24 - DNA, 20 bp, Artificial Sequence
  TCL_F1
cagtccaccaacacaacgtg

SEQ ID NO: 25 - DNA, 20 bp, Artificial Sequence
  TCL_R1
cccaagtgtcgctttgcatc

SEQ ID NO: 26 - DNA, 20 bp, Artificial Sequence
  TCL_F2
ccaacaaaggagccgcaatc

SEQ ID NO: 27 - DNA, 20 bp, Artificial Sequence
  TCL_R2
gcacatgtttggtgggtgtc

SEQ ID NO: 28 - DNA, 20 bp, Artificial Sequence
  TCL_F3
gacacccaccaaacatgtgc

SEQ ID NO: 29 - DNA, 20 bp, Artificial Sequence
  TCL_R3
gccaaacggaggtctggatt

SEQ ID NO: 30 - DNA, 20 bp, Artificial Sequence
  TCL_F4
aatccagacctccgtttggc

SEQ ID NO: 31 - DNA, 20 bp, Artificial Sequence
  TCL_R4
tcaatacctcgagcgcagac

SEQ ID NO: 32 - DNA, 20 bp, Artificial Sequence
  TCL_F5
tgtctgcgctcgaggtattg

SEQ ID NO: 33 - DNA, 20 bp, Artificial Sequence
  TCL_R5
tttgttcaggggtggtgtcc

SEQ ID NO: 34 - DNA, 20 bp, Artificial Sequence
  TCL_F6
tctgacgacaaaccggacac

SEQ ID NO: 35 - DNA, 20 bp, Artificial Sequence
  TCL_R6
agccgctgggattacttcac

SEQ ID NO: 36 - DNA, 20 bp, Artificial Sequence
  TCL_F7
ttcctagcaacccgctttca

SEQ ID NO: 37 - DNA, 20 bp, Artificial Sequence
  TCL_R7
agcggttttcttttccgtcg

SEQ ID NO: 38 - DNA, 20 bp, Artificial Sequence
  TCL_F8
cgacggaaaagaaaaccgct

SEQ ID NO: 39 - DNA, 19 bp, Artificial Sequence
  TCL_R8
actacggctactggggttc

SEQ ID NO: 40 - DNA, 20 bp, Artificial Sequence
  TCL_F9
ccccagtagccgtagttgac

SEQ ID NO: 41 - DNA, 20 bp, Artificial Sequence
  TCL_R9
tcatgccggagattttgcct

SEQ ID NO: 42 - DNA, 20 bp, Artificial Sequence
  TCL_F10
aggcaaaatctccggcatga

SEQ ID NO: 43 - DNA, 20 bp, Artificial Sequence
  TCL_R10
agatgagccgtcagcaaaca

SEQ ID NO: 44 - DNA, 20 bp, Artificial Sequence
  TCL_F11
tgtttgctgacggctcatct

SEQ ID NO: 45 - DNA, 20 bp, Artificial Sequence
  TCL_R11
tgcgtttgctctgtcgtagt

SEQ ID NO: 46 - DNA, 20 bp, Artificial Sequence
  TCL_F12
actacgacagagcaaacgca

SEQ ID NO: 47 - DNA, 20 bp, Artificial Sequence
  TCL_R12
atagagcgcaagccgtagac

SEQ ID NO: 48 - DNA, 20 bp, Artificial Sequence
  TCL_F13
gtctacggcttgcgctctat

SEQ ID NO: 49 - DNA, 20 bp, Artificial Sequence
  TCL_R13
gccagtaacaccatgtccca

SEQ ID NO: 50 - DNA, 20 bp, Artificial Sequence
  TCL_F14
tgggacatggtgttactggc

SEQ ID NO: 51 - DNA, 20 bp, Artificial Sequence
  TCL_R14
aacgctgttacgcggttttc

SEQ ID NO: 52 - DNA, 20 bp, Artificial Sequence
  TCL_F15
gaaaaccgcgtaacagcgtt

SEQ ID NO: 53 - DNA, 20 bp, Artificial Sequence
  TCL_R15
ctggcatcgtgttgtgtgtg

SEQ ID NO: 54 - DNA, 20 bp, Artificial Sequence
  TCL_F16
cacacacaacacgatgccag

SEQ ID NO: 55 - DNA, 20 bp, Artificial Sequence
  TCL_R16
tagacccgcactaccacact

SEQ ID NO: 56 - DNA, 20 bp, Artificial Sequence
  TCL_F17
agtgtggtagtgcgggtcta

SEQ ID NO: 57 - DNA, 20 bp, Artificial Sequence
  TCL_R17
ttttatgaccacgtccgcca

SEQ ID NO: 58 - DNA, 20 bp, Artificial Sequence
  TCL_F18
tggcggacgtggtcataaaa

SEQ ID NO: 59 - DNA, 20 bp, Artificial Sequence
  TCL_R18
ctgcagctcctctggtttca

SEQ ID NO: 60 - DNA, 20 bp, Artificial Sequence
  TCL_F19
tgaaaccagaggagctgcag

SEQ ID NO: 61 - DNA, 20 bp, Artificial Sequence
  TCL_R19
gccgaagctattgtgagggt

SEQ ID NO: 62 - DNA, 20 bp, Artificial Sequence
  TCL_F20
accctcacaatagcttcggc

SEQ ID NO: 63 - DNA, 20 bp, Artificial Sequence
  TCL_R20
cggaaaaacaacagccgagg

SEQ ID NO: 64 - DNA, 20 bp, Artificial Sequence
  TCL_F21
cctcggctgttgtttttccg

SEQ ID NO: 65 - DNA, 20 bp, Artificial Sequence
  TCL_R21
tgggtcagtaggtgcgagta

SEQ ID NO: 66 - DNA, 20 bp, Artificial Sequence
  TCL_F22
tactcgcacctactgaccca

SEQ ID NO: 67 - DNA, 20 bp, Artificial Sequence
  TCL_R22
ggagccaaccgaggataagg

SEQ ID NO: 68 - DNA, 20 bp, Artificial Sequence
  TCL_F23
tggcatgggttcgtcctttt

SEQ ID NO: 69 - DNA, 20 bp, Artificial Sequence
  TCL_R23
ggtacctttcgcaatgacgc

SEQ ID NO: 70 - DNA, 20 bp, Artificial Sequence
  TCL_F24
gcgtcattgcgaaaggtacc

SEQ ID NO: 71 - DNA, 20 bp, Artificial Sequence
  TCL_R24
accctccgccattgttgaat

SEQ ID NO: 72 - DNA, 20 bp, Artificial Sequence
  TCL_F25
attccaaccatcgcggttct

SEQ ID NO: 73 - DNA, 20 bp, Artificial Sequence
  TCL_R25
ttcgccttgataccagcgtt

SEQ ID NO: 74 - DNA, 20 bp, Artificial Sequence
  TCL_F26
acgctggtatcaaggcgaaa

SEQ ID NO: 75 - DNA, 20 bp, Artificial Sequence
  TCL_R26
atccaggagttaacgtcggc

SEQ ID NO: 76 - DNA, 20 bp, Artificial Sequence
  TCL_F27
gccgacgttaactcctggat

SEQ ID NO: 77 - DNA, 21 bp, Artificial Sequence
  TCL_R27
aggtcagaacaagggaggcta

SEQ ID NO: 78 - DNA, 20 bp, Artificial Sequence
  Assay 1 membrane primer 1
catctacctctcccatactc

SEQ ID NO: 79 - DNA, 23 bp, Artificial Sequence
  Assay 1 membrane primer 2
actgcttccaaaactgattacct

SEQ ID NO: 80 - DNA, 20 bp, Artificial Sequence
  Assay 1 membrane primer 3
gggcgtaaagagaatgtaag

(19) United States
(12) Patent Application Publication
(10) Pub. No.: US 2022/0347284 A1
(43) Pub. Date: Nov. 3, 2022

(54) NOVEL FISH CORONAVIRUS

(71) Applicant: Pharmaq AS, Overhalla (NO)

(72) Inventors: Stian NYLUND, Strusshamn (NO); Liv SANDLUND, Loddefjord (NO); Arnfinn L. OKLAND, Erdal (NO)

(21) Appl. No.: 17/433,407

(22) PCT Filed: Feb. 4, 2020
(86) PCT No.: PCT/EP2020/052688

§ 371 Date: Aug. 24, 2021

(30) Foreign Application Priority Data
Feb. 5, 2019 (EP) 19155627.3

(51) Int. Cl. A61K 39/12; C07K 14/005; C12N 7/00

(52) U.S. Cl. CPC A61K 39/12; C07K 14/005; C12N 7/00; C12N 2770/20021; C12N 2770/20022; C12N 2770/20034

(57) ABSTRACT

The invention relates to a novel fish virus, indicated to be a Coronavirus, which causes mortality in fish, and to methods of detecting said virus in fish, and related uses.

### FIELD OF THE INVENTION

The invention inter alia relates to a novel fish virus, indicated to be a Coronavirus, which causes mortality in fish, and to methods of detecting said virus in fish and protecting fish from infection by said virus, and related (re)agents and uses.

### BACKGROUND OF THE INVENTION

Fish are a major source of food and fish farming has become an important industry, particularly as rates of wild fish capture are flat or declining due to overfishing and loss of habitat. Examples of fish that are farmed include Atlantic salmon (Salmo salar) and lumpsucker (Cyclopterus lumpus).

However, infectious diseases in aquaculture threaten fish production and may also impact wild fish populations. For example, heart and skeletal muscle inflammation (HSMI) is known to be a frequently fatal disease of farmed Atlantic salmon. Affected fish often show reduced appetite prior to abnormal swimming behaviour and in some cases, sudden death. Usually, no external lesions are recognized. At autopsy, the heart often appears pale and somewhat loose. In some cases, the pericardial sac is filled with blood. Histological examinations indicate that most fish in affected net cages show severe lesions, although they seemingly appear to be healthy. First recognized in Norway in 1999 (Kongtorp et al., J Fish Dis 27, 2004), HSMI was subsequently implicated in several outbreaks in other farms in Norway and the United Kingdom. It is believed that piscine reovirus (PRV), a fish reovirus belongs to the family Reoviridae, subfamily Spinareovirinae, is the likely causative agent of HSMI (Kibenge et al., Virol J. 10, 2013). Since 1999 there has been an increasing number of outbreaks and the disease is considered to have a detrimental economic impact on the salmon farming industry.

Cardiomyopathy syndrome (CMS) is a severe cardiac disease affecting primarily large Atlantic salmon in the second year in seawater close to harvest. Affected fish may suddenly die without showing outward signs of disease, or may show symptoms such as abnormal swimming behaviour and anorexia. The disease was first recognized in farmed Atlantic salmon in Norway in 1985 and subsequently in farmed salmon in the Faroe Islands, the United Kingdom and Ireland. CMS has also been described in wild Atlantic salmon in Norway. In 2010, a double-stranded RNA virus of the Totiviridae family, named piscine myocarditis virus (PMCV), was described as the causative agent of CMS (Haugland et al, J. Virol, 85, 2011). PMCV is considered one of the largest problems in Atlantic salmon production, leading to major financial losses for salmon producing companies.

Disease challenges in the production of lumpfish (Cyclopterus lumpus) have to a certain degree been dominated by bacterial infections. Among these, Aeromonas salmonicida subsp. (atypical furunculosis), Pasteurella sp., Vibrio anguillarum and Tenacibaculum sp. have been the most significant species. The application of targeted vaccination programs, systematic monitoring of disease and improvements in production has led to a gradual decrease of the number of cases of atypical furuncolosis, vibriosis and pasteurellosis. However, several species of viruses have been detected in wild lumpfish, including viral hemorrhagic septicemia (VHSV) (Guõmundsd\acute{t}tir et al, J. Fish Dis, 42, 2019), viral nervous necrosis (VNN) and a novel ranavirus. Recently, a virus affecting farmed lumpfish has been identified: lumpfish flavivirus (LFV/CLuV) (Skoge et al, Arch Virol, 163, 2018). LFV/CLuV shows low but distinct similarity to the unassigned Tamana bat virus (TABV). LFV/CLuV was found to be present in all kinds of lumpfish tissues of affected fish, but pathology was primarily observed in the liver and kidneys. The virus is associated with serious disease in lumpfish. After LFV/CLuV was characterized, mapping the distribution of the virus and its association with disease has shown that it is widespread, with a relatively high associated prevalence.

Certain fish farms currently experience high mortality—for example up to 80% in some lumpsucker populations—despite real-time RT-PCR and histology failing to find any known pathogens in the fish.

Thus, there is an ongoing need to identify further pathogens which infect and which kill fish, particularly farmed lumpsucker fish. Further, there is an ongoing need for methods of monitoring the production of farmed fish for the presence of infection by pathogens, to avoid outbreaks of infection and potentially to treat infected fish.

### SUMMARY OF THE INVENTION

The present invention has surprisingly found a novel virus in lumpsucker fish, herein termed Cyclopterus lumpus Coronavirus (CLuCV). The length and organization of the genome, together with sequence analyses, indicate that CLuCV is a Torovirus, in the Coronaviridae family, with the Berne virus as its closest relative. Viruses in the Torovirus genus typically affect the intestine, and can lead to severe diarrhea in affected animals. The virus disclosed herein is the first Torovirus identified in fish.

Accordingly, one aspect of the invention provides a nucleic acid comprising at least one open reading frame (ORF) sequence selected from the group consisting of ORF-1, ORF-2, ORF-3, ORF-4 and ORF-5; wherein
ORF-1 is at least 80% identical to the nucleic acid sequence of SEQ ID NO:1,
ORF-2 is at least 80% identical to the nucleic acid sequence of SEQ ID NO:2,
ORF-3 is at least 80% identical to the nucleic acid sequence of SEQ ID NO:3,
ORF-4 is at least 80% identical to the nucleic acid sequence of SEQ ID NO:4, and
ORF-5 is at least 80% identical to the nucleic acid sequence of SEQ ID NO:5.

Another aspect of the invention provides a nucleic acid, wherein (a) the sequence of said nucleic acid is complementary to any one of SEQ ID NO:1 to SEQ ID NO:5; and/or (b) the sequence of said nucleic acid is complementary to SEQ ID NO:6.

Another aspect of the invention provides a viral polypeptide comprising an amino acid sequence that is at least 80%, at least 90%, or at least 95% identical to any one of SEQ ID NOs 7-11, or that is any one of SEQ ID NOs 7-11 or a conservatively substituted variant thereof.

Another aspect of the invention provides a virus that infects and is capable of killing lumpsucker fish (Cyclopterus lumpus), wherein the virus genome comprises a nucleic acid sequence disclosed herein, wherein said nucleic acid sequence contains the base uracil (U) instead of the base thymine (T), and/or wherein the virus comprises a viral polypeptide comprising an amino acid sequence that is at least 80%, at least 90%, or at least 95% identical to any one of SEQ ID NOs 7-11, or that is any one of SEQ ID NOs 7-11 or a conservatively substituted variant thereof.

Another aspect of the invention provides an oligonucleotide primer which comprises a sequence of at least 9 nucleotides, wherein said sequence is complementary to a nucleic acid sequence which is comprised within the genome of the virus disclosed herein.

Another aspect of the invention provides an oligonucleotide primer which comprises (a) a sequence of at least 9 consecutive nucleotides, wherein said sequence is complementary to a nucleic acid sequence which is comprised within the genome of the virus disclosed herein, (b) comprises at least 9 consecutive nucleotides of a sequence which is, or which is complementary to, a portion of a reference nucleic acid sequence selected from the group consisting of SEQ ID NO:1, SEQ ID NO:2, SEQ ID NO:3, SEQ ID NO:4, SEQ ID NO:5 or SEQ ID NO:6, or (c) comprises at least 9 consecutive nucleotides of a sequence which is at least 80% identical to a sequence which is, or which is complementary to, a sequence selected from the group consisting of SEQ ID NO:12 to SEQ ID NO:80; preferably with the proviso that said oligonucleotide primer does not comprise a sequence selected from the group consisting of SEQ ID NO:21 to SEQ ID NO:23.

Another aspect of the invention provides a method for detecting a virus that infects and is capable of killing fish, comprising the steps of:
(a) contacting a nucleic acid extracted from a biological sample of a fish with at least one oligonucleotide primer to form a mixture, wherein the at least one oligonucleotide primer is complementary to a nucleic acid sequence which is comprised within the genome of the virus disclosed herein, and
(b) determining whether upon subjecting the mixture of a) to amplification an amplification product is present, wherein the presence of amplification product indicates the presence of RNA associated with the virus, and hence the presence of the virus in the biological sample.

Another aspect of the invention provides a method for detecting a virus that infects and is capable of killing fish, comprising the steps of:
(a) sequencing a nucleic acid extracted from a biological sample of a fish, and
(b) comparing the resulting nucleic acid sequence with a nucleic acid sequence which is, or which is complementary to, a reference sequence selected from the group consisting of SEQ ID NO:1, SEQ ID NO:2, SEQ ID NO:3, SEQ ID NO:4, SEQ ID NO:5 and SEQ ID NO:6, wherein an at least 80% sequence identity between the two sequences indicates the presence of the virus in the biological sample.

Another aspect of the invention provides a method for detecting a virus that infects and is capable of killing fish, comprising the steps of:
(a) sequencing a nucleic acid extracted from a biological sample of a fish, and
(b) translating the resulting nucleic acid sequence into an amino acid sequence or translating a nucleic acid sequence complementary to said resulting nucleic acid sequence into an amino acid sequence, and
(c) comparing the resulting amino acid sequence with a reference sequence selected from the group consisting of SEQ ID NOs 7-11, wherein an at least 80% sequence identity between the two sequences indicates the presence of the virus in the biological sample.

Another aspect of the invention provides an antibody that binds a polypeptide, wherein the polypeptide is encoded by a nucleic acid sequence which is comprised within the genome of the virus disclosed herein, and/or wherein the polypeptide comprises an amino acid sequence that is at least 80%, at least 90%, or at least 95% identical to any one of SEQ ID NOs 7-11, or that is any one of SEQ ID NOs 7-11 or a conservatively substituted variant thereof.

Another aspect of the invention provides a kit for detecting a virus in a biological sample from fish, wherein the kit comprises an oligonucleotide primer disclosed herein and/or an antibody disclosed herein.

Another aspect of the invention provides an antibody for use in treating fish infected with a virus disclosed herein.

Another aspect of the invention provides a use of the virus disclosed herein for producing a vaccine.

Another aspect of the invention provides a vaccine comprising:
(i) a nucleic acid sequence which is comprised within the genome of the virus disclosed herein;
(ii) a nucleic acid sequence disclosed herein;
(iii) a viral polypeptide encoded by a nucleic acid sequence which is comprised within the genome of the virus disclosed herein;
(iv) a viral polypeptide comprising an amino acid sequence that is at least 80%, at least 90%, or at least 95% identical to any one of SEQ ID NOs 7-11, or that is any one of SEQ ID NOs 7-11 or a conservatively substituted variant thereof; or
(v) a virus disclosed herein.

Yet another aspect of the invention provides an interfering RNA (iRNA) molecule for use in treating fish infected with a virus, wherein the iRNA molecule comprises at least 12 consecutive nucleotides of, or complimentary to, a nucleic acid sequence comprised within the genome of the virus disclosed herein.

### BRIEF DESCRIPTION OF THE FIGURES

FIG. 1: A schematic of the sequence of CLuCV identified herein, which is 24,613 nucleotides long and contains five possible open reading frames (ORFs).

FIG. 2: CLuCV genome nucleotide sequence.

FIG. 3: CLuCV ORF-1 nucleotide sequence.

FIG. 4: CLuCV ORF-2 nucleotide sequence.

FIG. 5: CLuCV ORF-3, ORF-4 and ORF-5 nucleotide sequences.

FIG. 6: CLuCV ORF-1 amino acid sequence.

FIG. 7: CLuCV ORF-2 amino acid sequence.

FIG. 8: CLuCV ORF-3, ORF-4 and ORF-5 amino acid sequences.

FIG. 9: Haematoxylin and Eosin stain of a whole section of affected lumpsucker, showing accumulation of fluid in the stomach (arrow).

FIG. 10: Haematoxylin and Eosin stain of a section of lumpsucker intestine showing accumulation of mucus, and cellular discharge (arrows).

FIG. 11: Haematoxylin and Eosin stain of a section of lumpsucker intestine showing accumulation of mucus (arrow), and cellular discharge.

FIG. 12: Haematoxylin and Eosin stain of a section of lumpsucker intestine showing accumulation of mucus (arrow).

### DETAILED DESCRIPTION OF THE INVENTION

### Definitions

In order for the present invention to be readily understood, several definitions of terms used in the course of the invention are set forth below.

As used herein, the term “lumpsucker” or “lumpfish” is intended to mean any species selected from the whole family of Cyclopteridae. The most preferred species according to the invention is Cyclopterus lumpus.

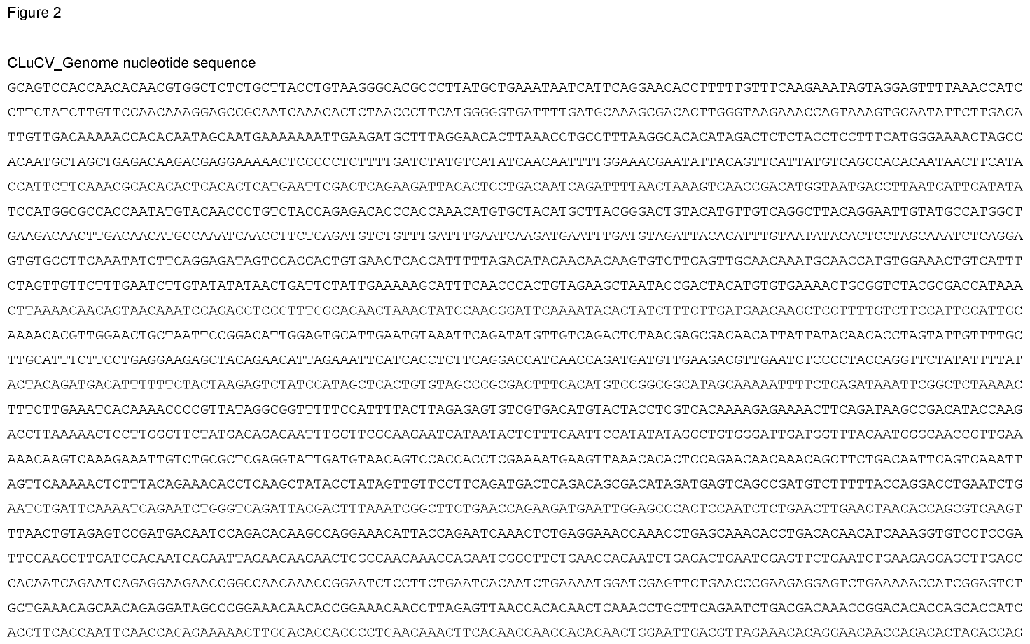

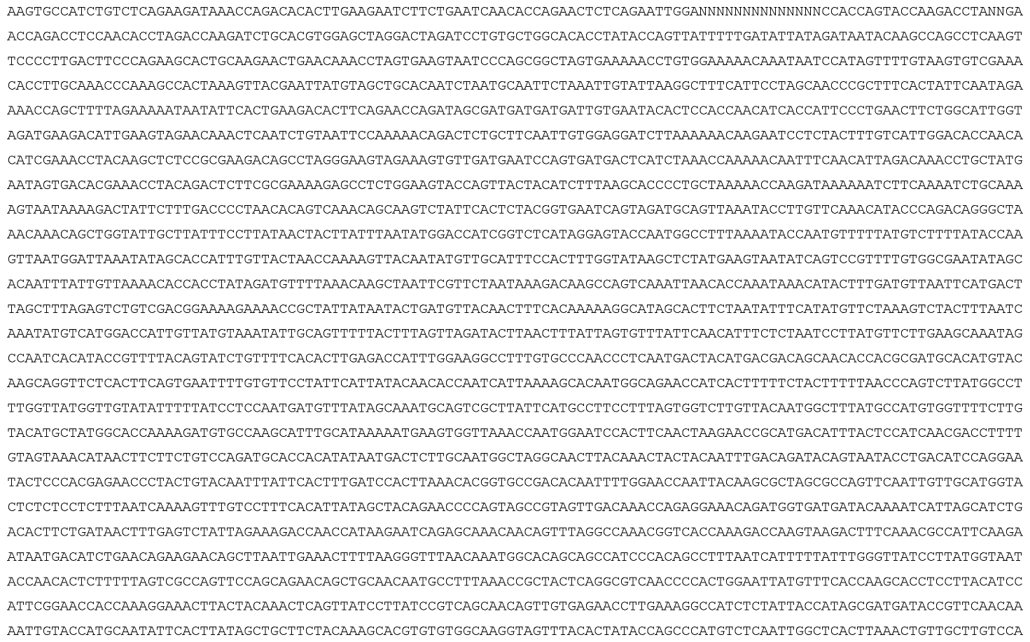

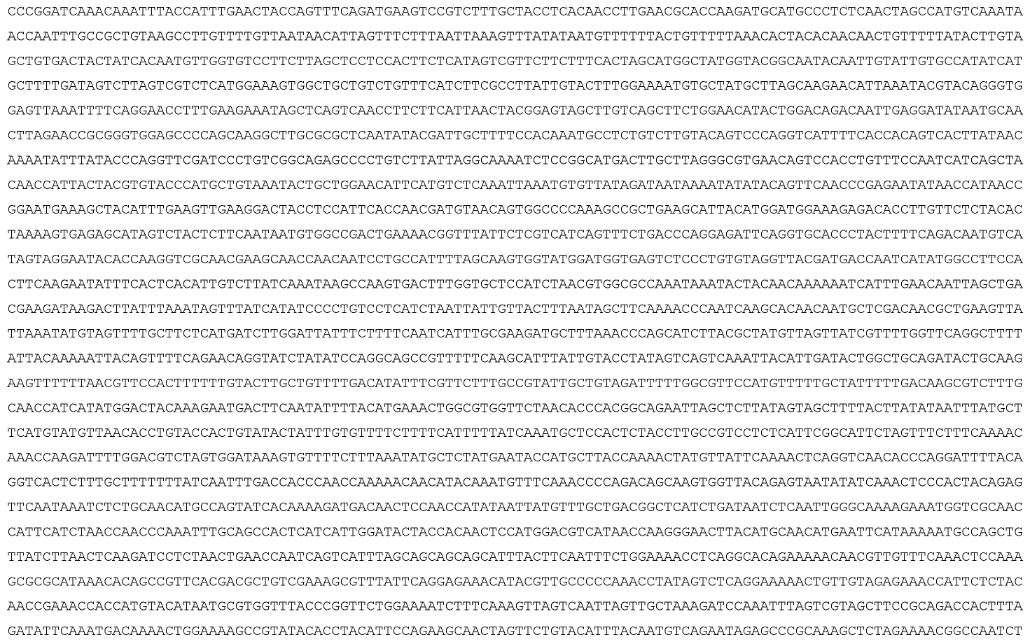

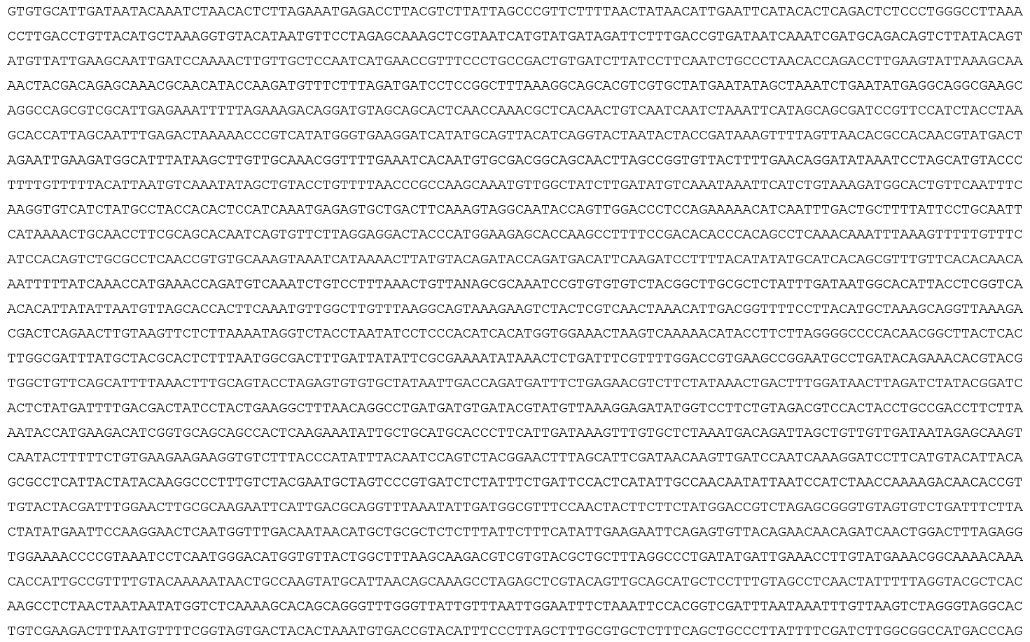

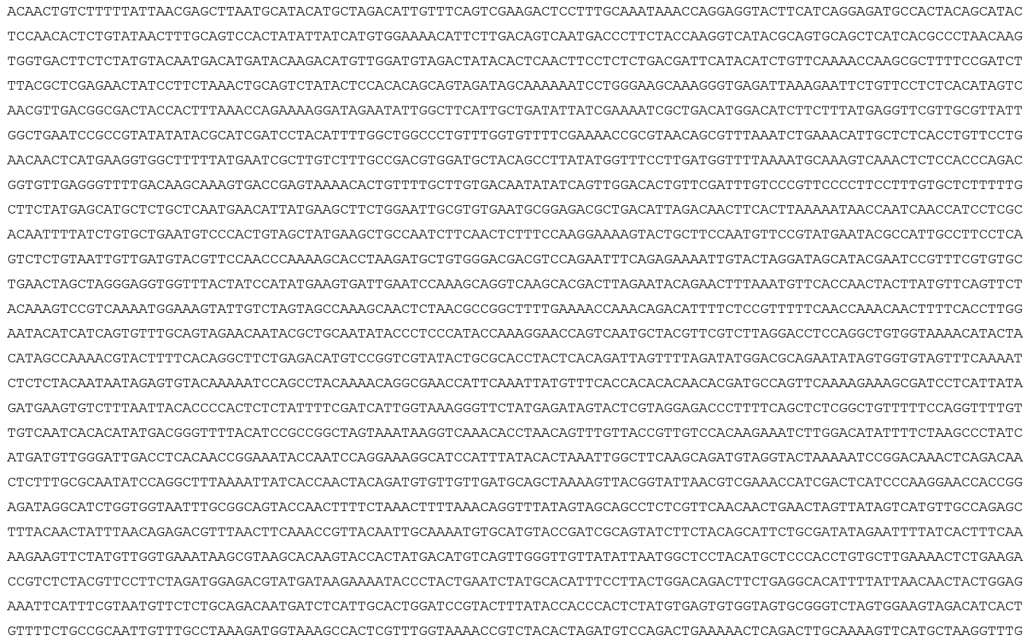

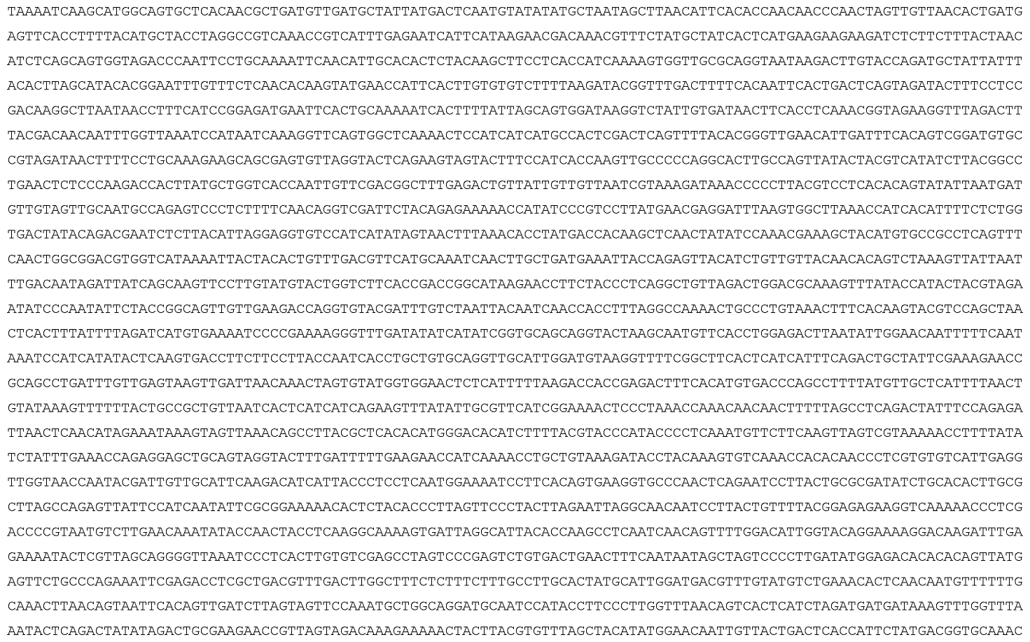

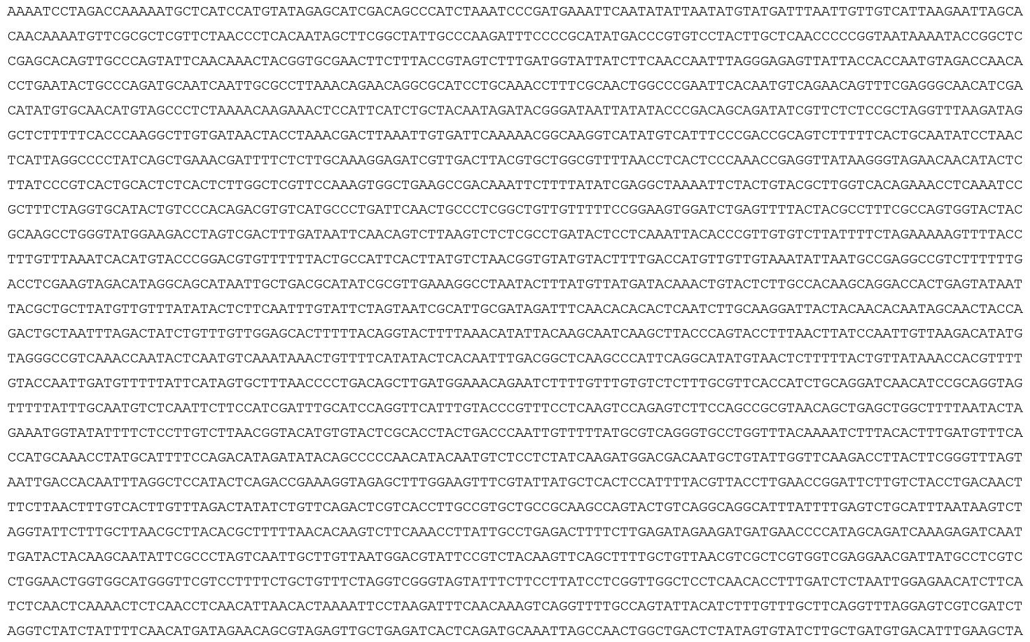

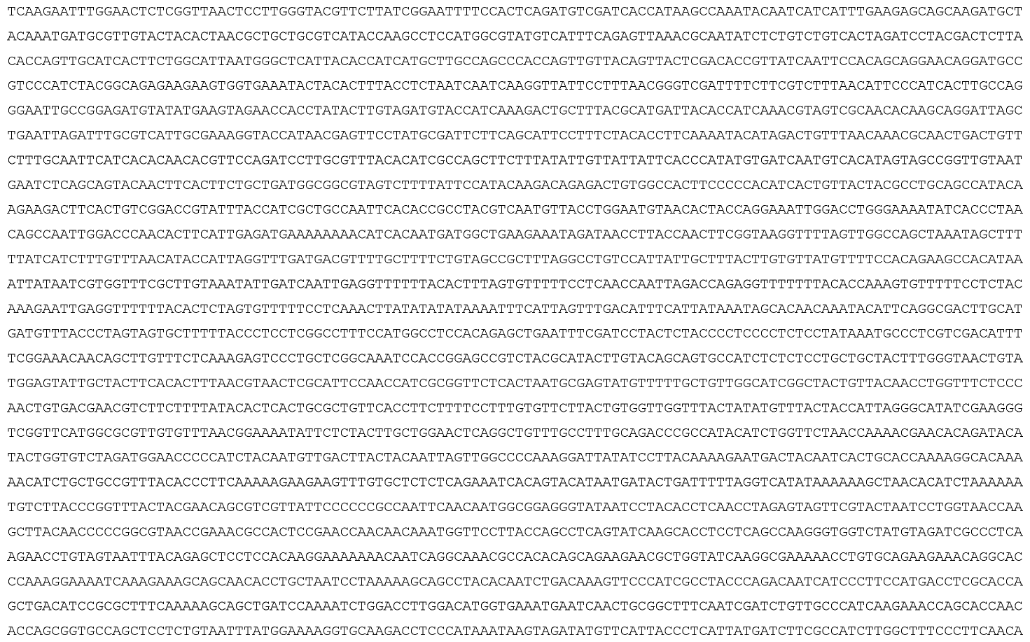

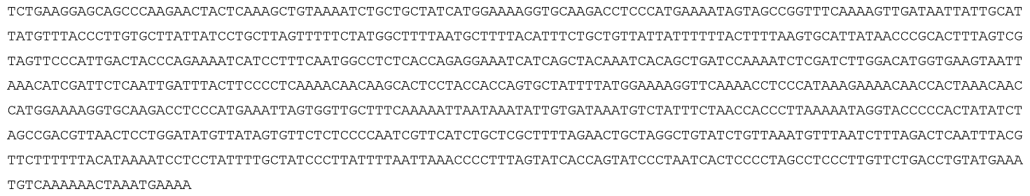

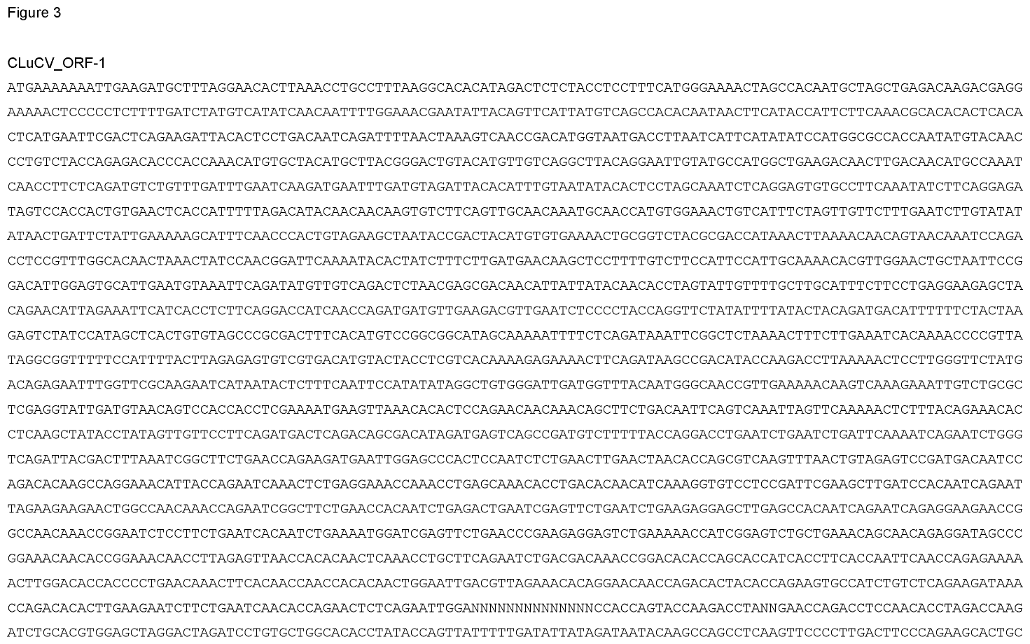

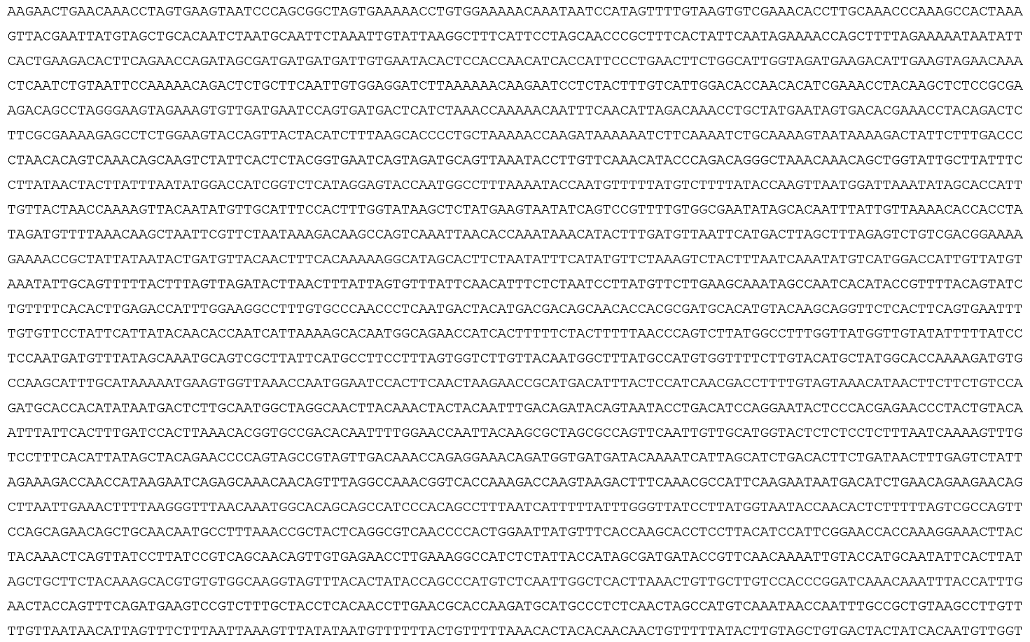

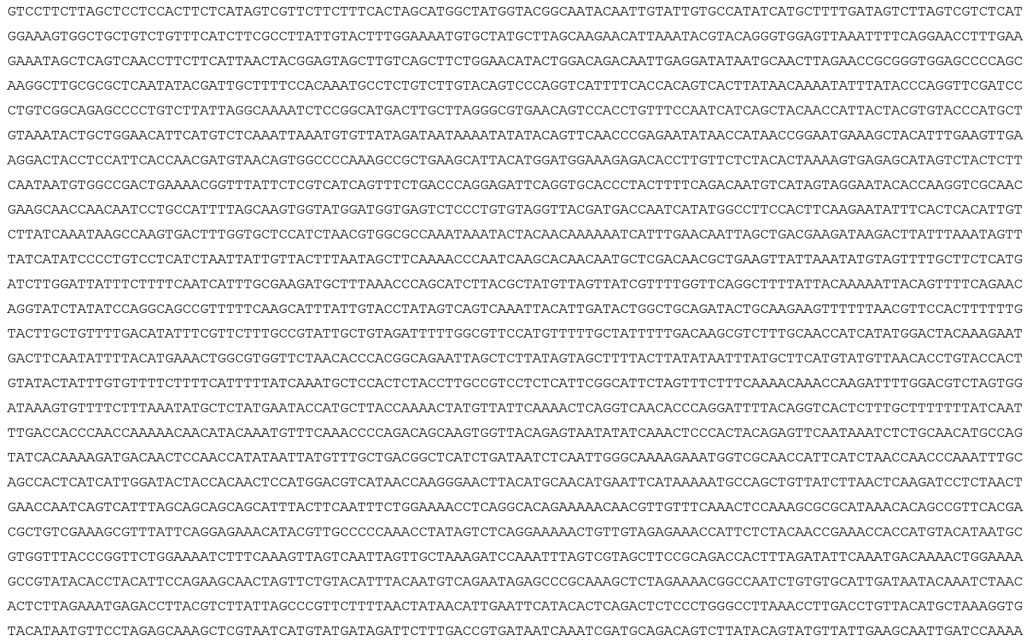

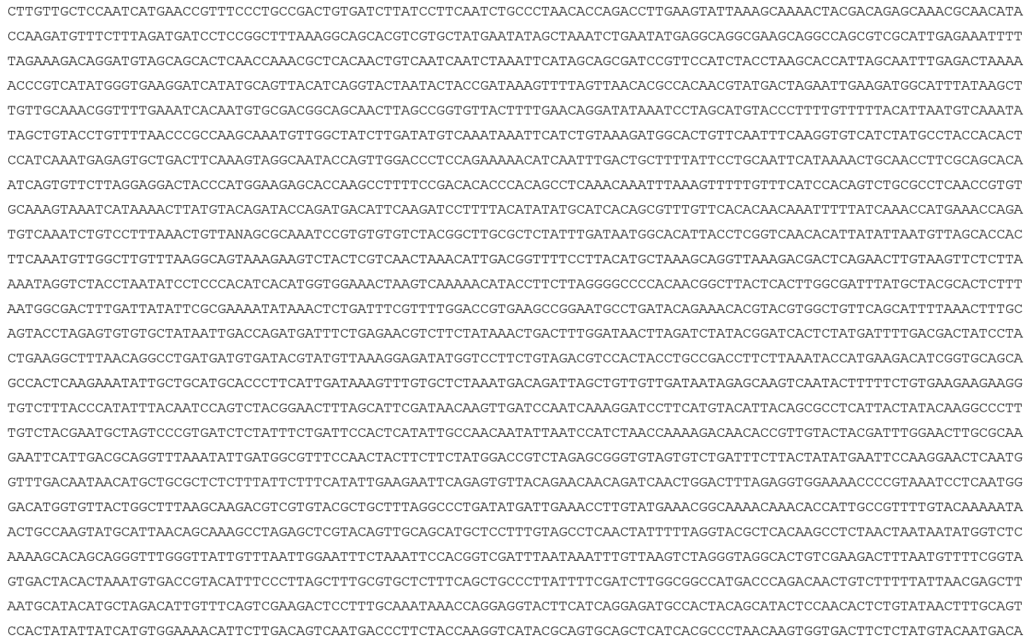

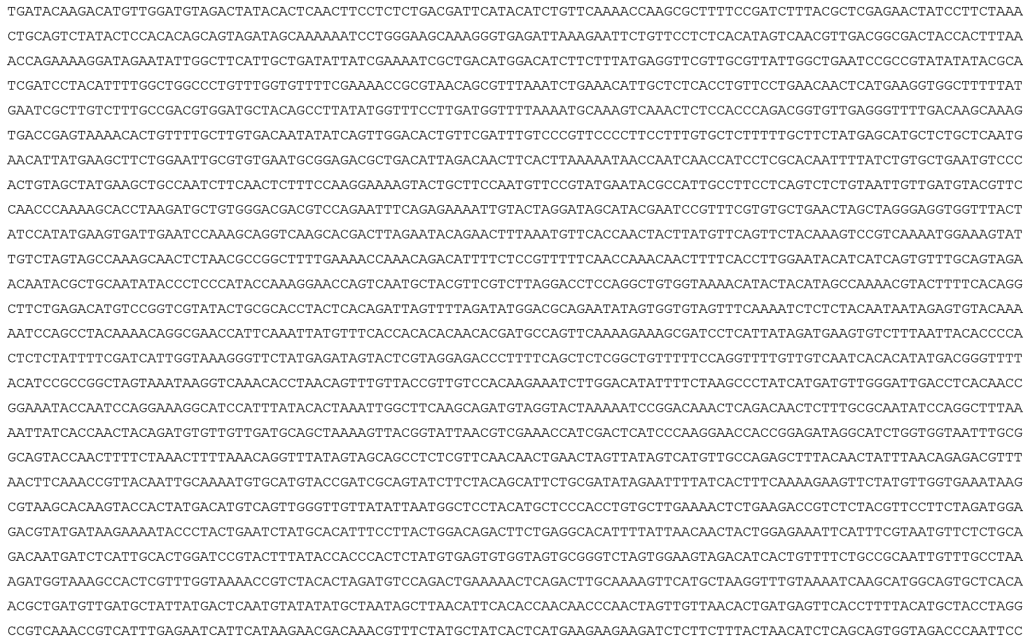

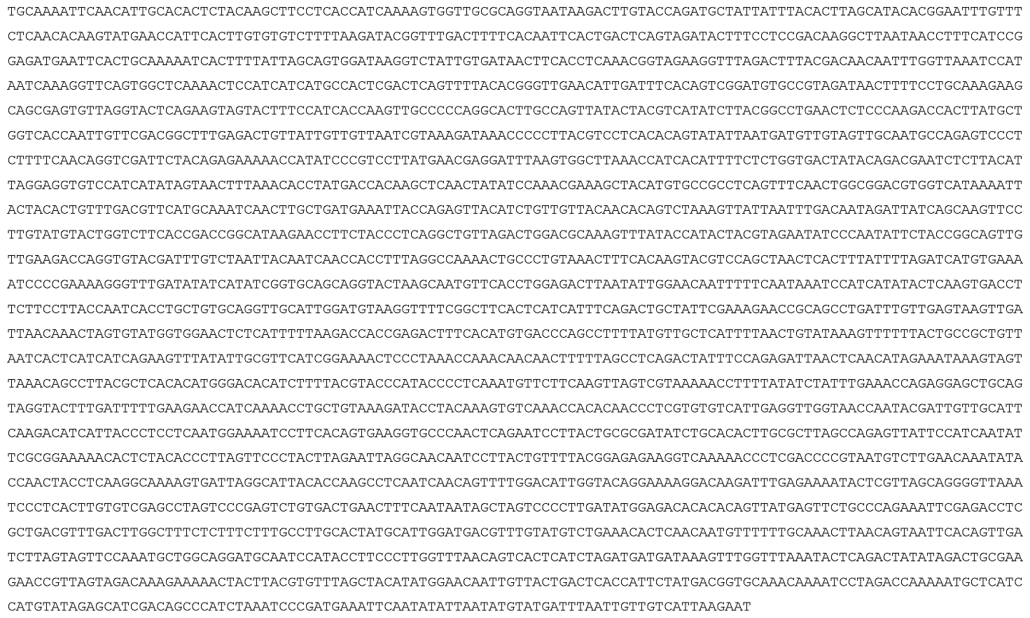

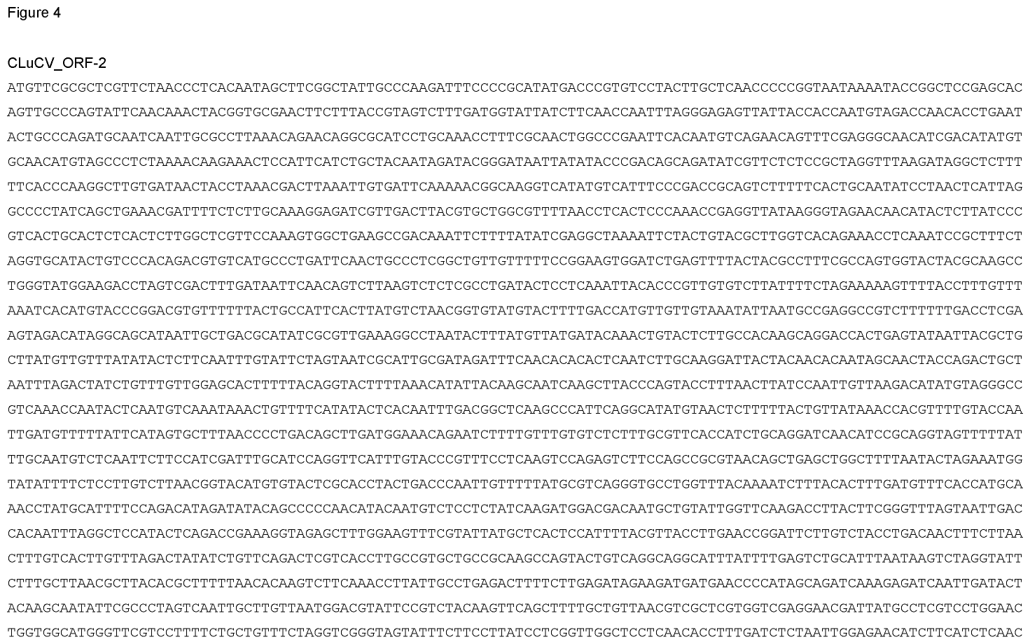

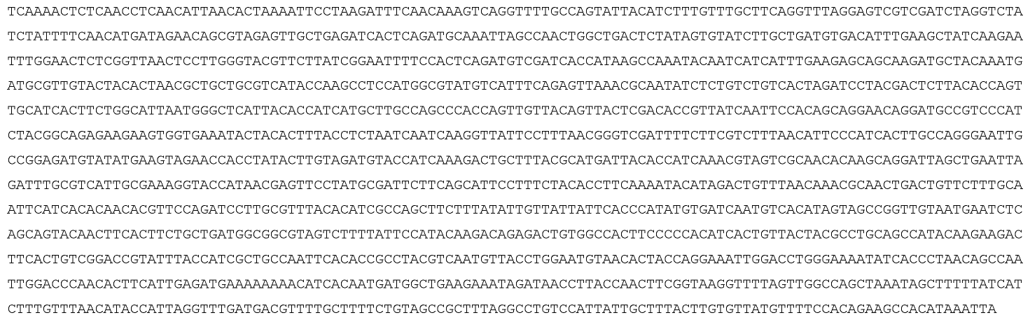

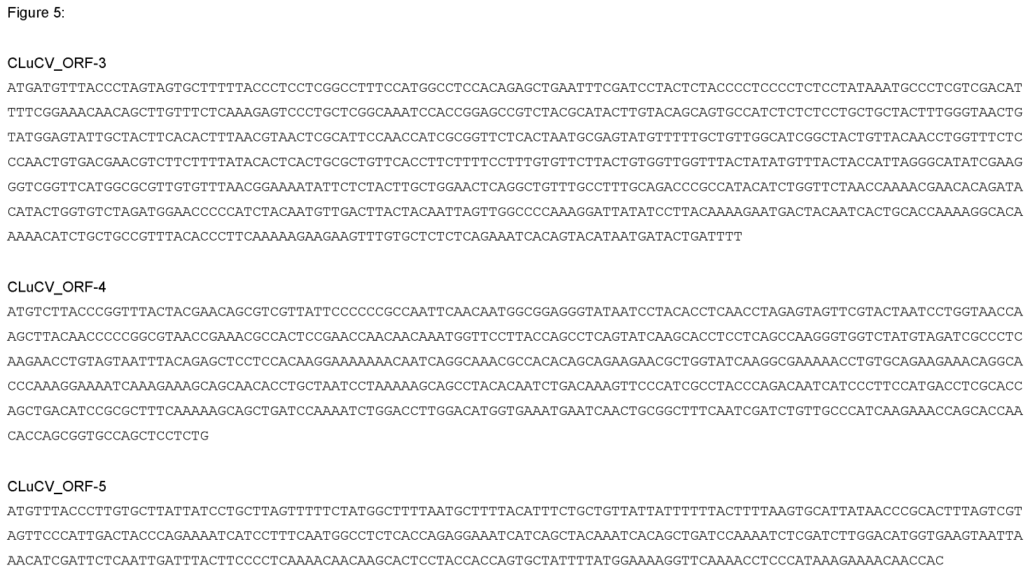

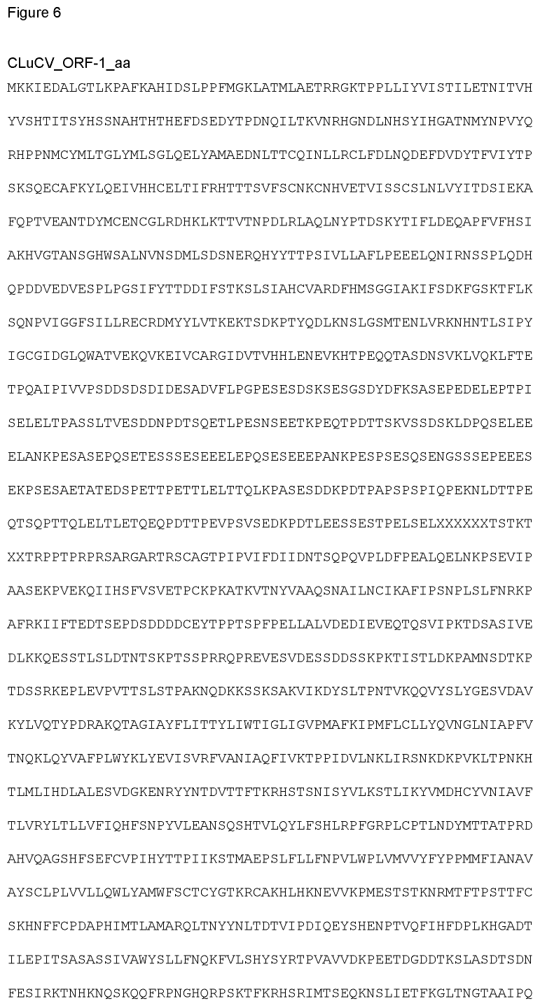

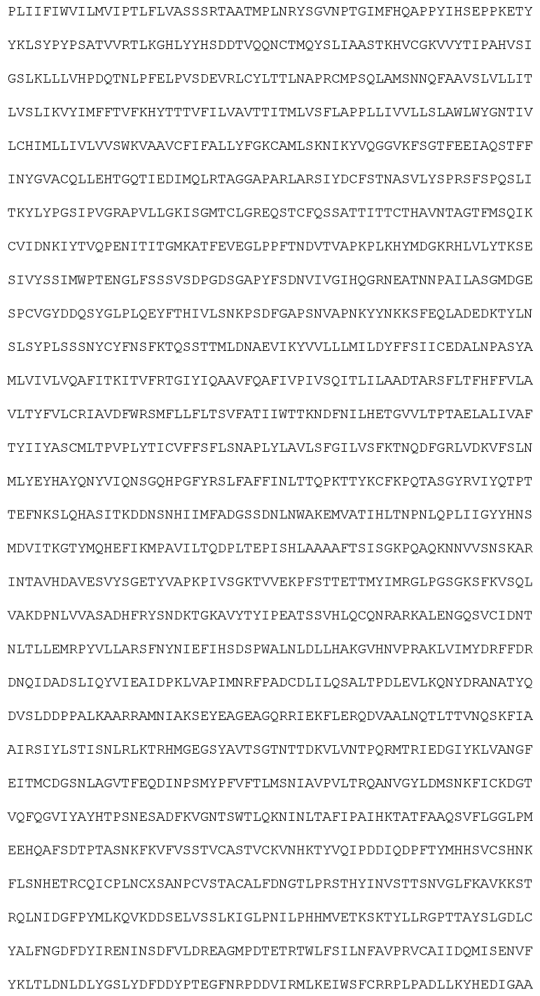

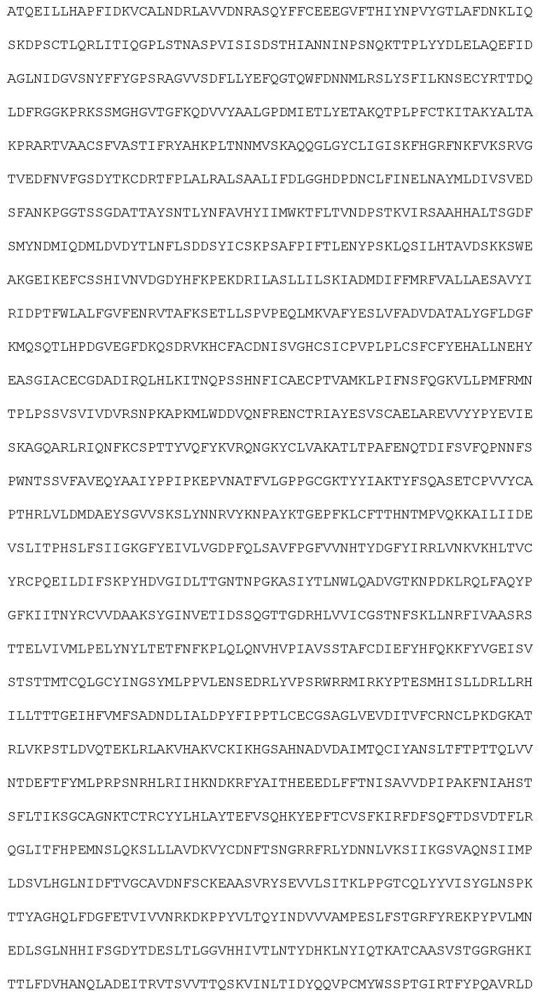

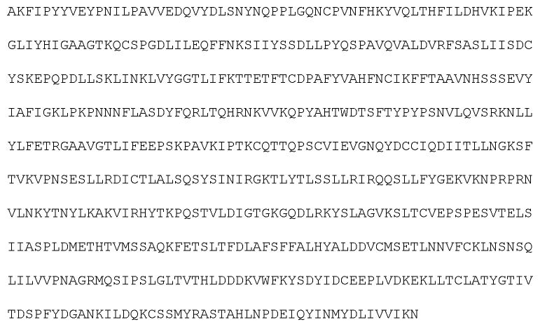

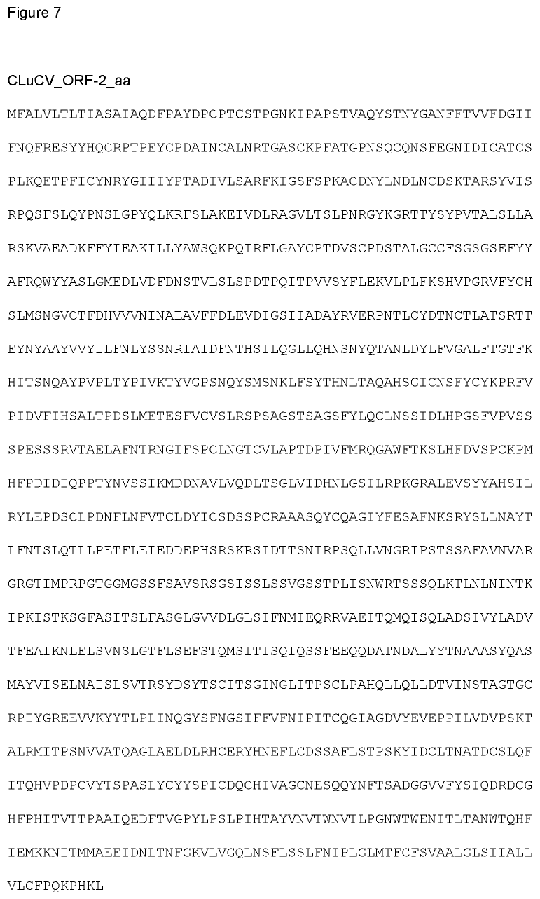

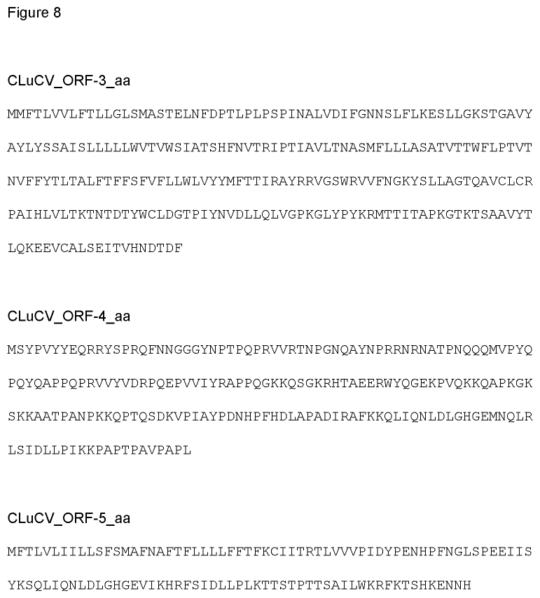

The term “nucleic acid” includes DNA molecules (e.g., cDNA or genomic DNA), RNA molecules (e.g., mRNA), analogues of DNA or RNA generated using nucleotide analogues (e.g., peptide nucleic acids and non-naturally occurring nucleotide analogues), and hybrids thereof. Thus, whereas the nucleic acid sequences provided in FIGS. 2 to 5 and SEQ ID NOs: 1 to 6 use the bases guanine, cytosine, adenine and thymine, embodiments of the invention relate to their corresponding RNA sequences which use the bases guanine, cytosine, adenine and uracil (i.e., with uracil instead of thymine), which RNA sequences are therefore also provided herein. The nucleic acid molecule can be single-stranded or double-stranded. Unless specified otherwise, the left-hand end of any single-stranded nucleic acid sequence discussed herein is the 5′ end. The direction of 5′ to 3′ addition of nascent RNA transcripts is the transcription direction.

The term “oligonucleotide” means a nucleic acid comprising 200 or fewer nucleotides. Oligonucleotides can be single stranded, e.g., for use as primers, cloning primers or hybridization probes, or they can be double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays.

As used herein, the term “oligonucleotide primer” or “primer” is to be understood to refer to a nucleic acid (e.g., of at least 9 nucleotides in length, and less than 60 nucleotides in length) suitable for directing an activity to a region of a nucleic acid, e.g. for amplification of a target nucleic acid sequence by the polymerase chain reaction (PCR), or for in situ hybridization.

As used herein, the term “complementary” in the context of nucleic acid sequences means nucleic acid sequences that form a double-stranded structure by matching base pairs (A to T (or U); and G to C). For example, the complementary nucleic acid sequence to G-T-A-C is C-A-T-G. Other examples of complementary nucleic acid sequences are the following:

Complementary nucleic acid sequence (e.g., in case the nucleic acid is DNA):

5′-ATTCGCTTAACGCAA-3′
3′-TAAGCGAATTGCGTT-5′

The corresponding complementary sequences wherein uracil is substituted for thymine (e.g., in case the nucleic acid is RNA):

5′-AUUCGCUUAACGCAA-3′
3′-UAAGCGAAUUGCGUU-5′

As used herein, the term “amino acid” refers to one of the 20 naturally occurring amino acids or any non-natural analogues. Preferably, the term “amino acid” refers to one of the 20 naturally occurring amino acids.

The terms “polypeptide” or “protein” mean a macromolecule composed of a sequence of amino acids. A protein can be a native protein, that is, a protein produced by a naturally-occurring and non-recombinant cell; or it can be produced by a genetically-engineered or recombinant cell, and comprise molecules having the amino acid sequence of the native protein, or molecules having deletions from, additions to, and/or substitutions of one or more amino acids of the native sequence. The terms also include amino acid polymers in which one or more amino acids are chemical analogues of a corresponding naturally-occurring amino acid polymer.

The term “sequence identity” indicates a quantitative measure of the degree of homology between two sequences, which can be nucleic acid (also termed nucleotide) sequences or amino acid sequences. If the two sequences to be compared are not of equal length, they must be aligned to give the best possible fit, allowing the insertion of gaps or alternatively, truncation at the ends of the nucleic acid sequences or amino acid sequences.

In the case of a nucleotide sequence, for example, the term “at least 80% identical” thus means that at least 80% of the nucleotides over the entire sequence can be aligned with identical nucleotides from another sequence. A specified percentage of nucleotides can be referred to as e.g. 80% identical, 85% identical, 90% identical, 95% identical, 99% identical or more over a specified region when compared and aligned for maximum correspondence. For example, a sequence which is 10 nucleotides in length, say GGGAAACCTT, can be 80% identical with a continuous sequence (e.g., GGGAAACCGG), or with a non-continuous sequence (e.g., GGGACCCCTT):

### 100% Identity Example

GGGAAACCTT
||||||||||
GGGAAACCTT

### 80% Identity Example

GGGAAACCTT
||||||||
GGGAAACCGG

### 80% Identity Example

GGGAAACCTT
|||| ||||
GGGACCCCTT

In case a base designated “N” is found on a particular position within the reference nucleotide sequence, sequence identity is given for any of the bases adenine (A), cytosine (C), guanine (G) and thymine (T) or uracil (U), at the corresponding position on the compared sequence. See the following identity example. The same applies to amino acid sequence comparisons, i.e., in case an amino acid designated “X” is found on a particular position within the reference amino acid sequence, sequence identity is given for any amino acid at the corresponding position on the compared sequence.

### 80% Identity Example

GGGAAANCCT
|||| ||||
GGGACCCCCT

The skilled person will acknowledge that various means for comparing sequences are available (see below).

As used herein, the term “conservatively substituted” in reference to an amino acid means that the amino acid can be substituted by another amino acid in its respective group, according to the following six groups: [1] Alanine (A), Serine (S), Threonine (T); [2] Aspartic acid (D), Glutamic acid (E); [3] Asparagine (N), Glutamine (Q); [4] Arginine (R), Lysine (K); [5] Isoleucine (I), Leucine (L), Methionine (M), Valine (V); and [6] Phenylalanine (F), Tyrosine (Y), Tryptophan (W). A “conservatively substituted variant” in reference to a polypeptide or protein means that any of the amino acids in said polypeptide or protein can be conservatively substituted as defined hereinabove.

As used herein, the term “antibody” refers to a glycoprotein comprising at least two heavy (H) chains and two light (L) chains inter-connected by disulfide bonds, or an antibody fragment (antigen binding portion) thereof. Each heavy chain is comprised of a heavy chain variable region (VH) and a heavy chain constant region (CH). The heavy chain constant region is comprised of three domains, CH1, CH2 and CH3. Each light chain is comprised of a light chain variable region (VL) and a light chain constant region (CL). The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host cells or factors, including various cells of the immune system (e.g., effector cells) and the first component (C1q) of the classical complement system. Antibodies of the invention include monoclonal antibodies (including full length monoclonal antibodies) and polyclonal antibodies, whole antibodies, chimeric antibodies, humanized antibodies, human antibodies or hybrid antibodies with dual or multiple antigen or epitope specificities, antibody fragments and antibody sub-fragments, e.g., Fab, Fab′, F(ab′)₂, fragments and the like, including hybrid fragments of any immunoglobulin or any natural, synthetic or genetically engineered protein that acts like an antibody by binding to a specific antigen to form a complex. Antibodies of the invention can also be Fc fusion proteins.

A “vector” is a nucleic acid that can be used to introduce another nucleic acid (or “construct”) linked to it into a cell. One type of vector is a “plasmid”, which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) integrate into the genome of a host cell upon introduction into the host cell and culturing under selective pressure, and thereby are replicated along with the host genome. A vector can be used to direct the expression of a chosen nucleic acid in a cell.

A “host cell” is a cell that can be used to express a nucleic acid, e.g., a nucleic acid disclosed herein. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Exemplary host cells include Chinese hamster ovary (CHO) cell lines or their derivatives. Typically, a host cell is a cultured cell that can be transformed or transfected with a polypeptide-encoding nucleic acid, which can then be expressed in the host cell. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The terms “treat” and “treatment” include therapeutic treatments, prophylactic treatments, and applications which reduce symptoms of a disorder or reduce the risk that a subject (e.g., a fish) will develop a disorder (e.g., symptoms of virus infection).

The term “vaccine” as used herein refers to a material that can produce an immune response that blocks the infectivity, either partially or fully, of an infectious agent, which in respect of the present invention is the virus affecting fish, e.g., lumpsuckers. Thus, when administering to a fish, the vaccine of the invention immunises the fish against the disease caused by the virus. The immunising component of the vaccine may be, e.g., DNA as in a DNA vaccine, RNA as in a RNA vaccine, a recombinant protein or fragment thereof according to the present invention, or a live or attenuated recombinant virus.

An “iRNA agent” (abbreviation for “interfering RNA agent”) as used herein, is an RNA agent which can down-regulate (reduce) the expression of a target gene, e.g., a protein encoded by ORF-1, ORF-2, ORF-3, ORF-4 or ORF-5. An iRNA agent may act by one or more mechanisms, including post-transcriptional cleavage of a target mRNA sometimes referred to in the art as “RNAi”, or pre-transcriptional or pre-translational mechanisms. An iRNA agent can be a double-stranded (ds) iRNA agent. An iRNA agent can also be a “small interfering RNA” (siRNA).

The terms “of the invention” or “according to the invention” as used herein are intended to refer to all aspects and embodiments of the invention disclosed and/or claimed herein. Conversely, any aspects, items or embodiments referred to herein as being “disclosed herein” or “described herein” are to be understood as being aspects, items or embodiments “of the invention” or “according to the invention”.

As used herein, the term “comprising” is to be construed as encompassing both “including” and “consisting of”, both meanings being specifically intended, and hence individually disclosed, embodiments according to the present invention.

As used herein, the articles “a” and “an” preceding an element or component are intended to be nonrestrictive regarding the number of instances (i.e. occurrences) of the element or component. Therefore, “a” or “an” is to be read to include one or at least one, and the singular word form of the element or component also includes the plural unless the number is obviously meant to be singular.

As used herein, the term “about” modifying the quantity of a substance, ingredient, component, or parameter employed refers to variation in the numerical quantity that can occur, for example, through typical measuring and handling procedures, e.g., liquid handling procedures used for making concentrates or solutions. Furthermore, variation can occur from inadvertent error in measuring procedures, differences in the manufacture, source, or purity of the ingredients employed to carry out the methods, and the like. In one embodiment, the term “about” means within 10% of the reported numerical value. In a more specific embodiment, the term “about” means within 5% of the reported numerical value.

### Virus Nucleic Acid Sequences and Viral Polypeptides

One aspect of the invention provides a nucleic acid comprising at least one open reading frame (ORF) sequence selected from the group consisting of ORF-1, ORF-2, ORF-3, ORF-4 and ORF-5; wherein
ORF-1 is at least 80% identical to the nucleic acid sequence of SEQ ID NO:1,
ORF-2 is at least 80% identical to the nucleic acid sequence of SEQ ID NO:2,
ORF-3 is at least 80% identical to the nucleic acid sequence of SEQ ID NO:3,
ORF-4 is at least 80% identical to the nucleic acid sequence of SEQ ID NO:4, and
ORF-5 is at least 80% identical to the nucleic acid sequence of SEQ ID NO:5.

In some embodiments,
ORF-1 is at least 85% identical to the nucleic acid sequence of SEQ ID NO:1,
ORF-2 is at least 85% identical to the nucleic acid sequence of SEQ ID NO:2,
ORF-3 is at least 85% identical to the nucleic acid sequence of SEQ ID NO:3,
ORF-4 is at least 85% identical to the nucleic acid sequence of SEQ ID NO:4, and
ORF-5 is at least 85% identical to the nucleic acid sequence of SEQ ID NO:5.

In preferred embodiments,
ORF-1 is at least 90% identical to the nucleic acid sequence of SEQ ID NO:1,
ORF-2 is at least 90% identical to the nucleic acid sequence of SEQ ID NO:2,
ORF-3 is at least 90% identical to the nucleic acid sequence of SEQ ID NO:3,
ORF-4 is at least 90% identical to the nucleic acid sequence of SEQ ID NO:4, and
ORF-5 is at least 90% identical to the nucleic acid sequence of SEQ ID NO:5.

In more preferred embodiments,
ORF-1 is at least 95% identical to the nucleic acid sequence of SEQ ID NO:1,
ORF-2 is at least 95% identical to the nucleic acid sequence of SEQ ID NO:2,
ORF-3 is at least 95% identical to the nucleic acid sequence of SEQ ID NO:3,
ORF-4 is at least 95% identical to the nucleic acid sequence of SEQ ID NO:4, and
ORF-5 is at least 95% identical to the nucleic acid sequence of SEQ ID NO:5.

In yet more preferred embodiments,
ORF-1 is at least 98% identical to the nucleic acid sequence of SEQ ID NO:1,
ORF-2 is at least 98% identical to the nucleic acid sequence of SEQ ID NO:2,
ORF-3 is at least 98% identical to the nucleic acid sequence of SEQ ID NO:3,
ORF-4 is at least 98% identical to the nucleic acid sequence of SEQ ID NO:4, and
ORF-5 is at least 98% identical to the nucleic acid sequence of SEQ ID NO:5.

In yet even more preferred embodiments,
ORF-1 is at least 99% identical to the nucleic acid sequence of SEQ ID NO:1,
ORF-2 is at least 99% identical to the nucleic acid sequence of SEQ ID NO:2,
ORF-3 is at least 99% identical to the nucleic acid sequence of SEQ ID NO:3,
ORF-4 is at least 99% identical to the nucleic acid sequence of SEQ ID NO:4, and
ORF-5 is at least 99% identical to the nucleic acid sequence of SEQ ID NO:5.

In particularly preferred embodiments,
ORF-1 is the nucleic acid sequence of SEQ ID NO:1,
ORF-2 is the nucleic acid sequence of SEQ ID NO:2,
ORF-3 is the nucleic acid sequence of SEQ ID NO:3,
ORF-4 is the nucleic acid sequence of SEQ ID NO:4, and
ORF-5 is the nucleic acid sequence of SEQ ID NO:5.

In particular embodiments, the nucleic acid disclosed herein comprises at least ORF-1, ORF-2, ORF-3 and/or ORF-4, according to any of their embodiments disclosed herein.

The sequence of the nucleic acid disclosed herein can be at least 80% identical to the virus genome according to SEQ ID NO:6. In some embodiments, the sequence of the nucleic acid disclosed herein is at least 85% identical to the virus genome according to SEQ ID NO:6. In preferred embodiments, the sequence of the nucleic acid disclosed herein is at least 90% identical to the virus genome according to SEQ ID NO:6. In more preferred embodiments, the sequence of the nucleic acid disclosed herein is at least 95% identical to the virus genome according to SEQ ID NO:6. In yet more preferred embodiments, the sequence of the nucleic acid disclosed herein is at least 98% identical to the virus genome according to SEQ ID NO:6. In yet even more preferred embodiments, the sequence of the nucleic acid disclosed herein is at least 99% identical to the virus genome according to SEQ ID NO:6. In particularly preferred embodiments, the sequence of the nucleic acid disclosed herein has 100% identity to the sequence of the virus genome according to SEQ ID NO:6 (CLuCV).

Also provided herein is a nucleic acid the sequence of which is complementary to the sequence of any of the nucleic acids disclosed herein.

The sequence of said nucleic acid can be complementary to ORF-1, ORF-2, ORF-3, ORF-4 or ORF-5, according to any of their embodiments disclosed herein. The sequence of the nucleic acid can also be complementary to a nucleic acid sequence which is at least 80% identical to, in some embodiments at least 85% identical to, in preferred embodiments at least 90% identical to, in more preferred embodiments at least 95% identical to, in yet more preferred embodiments at least 98% identical to, in yet even more preferred embodiments at least 99% identical to, and in particularly preferred embodiments 100% identical to the sequence of the virus genome according to SEQ ID NO:6 (CLuCV).

Accordingly, the invention also provides a nucleic acid, wherein (a) the sequence of said nucleic acid is complementary to any one of SEQ ID NO:1 to SEQ ID NO:5; and/or (b) the sequence of said nucleic acid is complementary to SEQ ID NO:6.

In preferred embodiments, the nucleic acid sequences disclosed herein are RNA nucleic acid sequences, i.e., they contain the base uracil (U) instead of the base thymine (T). Accordingly, the viruses disclosed herein contain their genetic information in the form of such RNA nucleic acid sequences.

The skilled person will further acknowledge that alterations of the nucleic acid sequence resulting in modifications of the amino acid sequence of the protein it codes may have little, if any, effect on the resulting three dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K); [5] Isoleucine (I), Leucine (L), Methionine (M), Valine (V); and [6] Phenylalanine (F), Tyrosine (Y), Tryptophan (W). (see, e.g., US Patent Publication 20100291549).

Preferably, ORFs 1-5 herein encode viral polypeptides comprising amino acid sequences of SEQ ID NOs: 7-11, respectively, or amino acid sequences that are at least 80% identical (e.g., at least 85% identical or at least 90% identical, or at least 91% identical, at least 92% identical, at least 93% identical, at least 94% identical, at least 95% identical, at least 96% identical, at least 97% identical, at least 98% identical, or at least 99% identical) to amino acid sequences SEQ ID NOs: 7-11, respectively. In certain embodiments, ORFs 1-5 encode viral polypeptides that are conservatively substituted variants of SEQ ID NOs 7-11, respectively, as described above.

Therefore, in another aspect, herein provided are viral polypeptides comprising amino acid sequences of SEQ ID NOs: 7-11, respectively, or amino acid sequences that are at least 80% identical (e.g., at least 85% identical or at least 90% identical, or at least 91% identical, at least 92% identical, at least 93% identical, at least 94% identical, at least 95% identical, at least 96% identical, at least 97% identical, at least 98% identical, or at least 99% identical) to amino acid sequences SEQ ID NOs: 7-11, respectively. In certain embodiments, the viral polypeptides are conservatively substituted variants of SEQ ID NOs 7-11, respectively, as described above. Vectors, e.g., plasmid vectors or viral vectors, comprising nucleic acid sequences encoding the viral polypeptides of the invention as described above, are also provided.

Protein and/or nucleic acid sequence identities (homologies) can be evaluated using any of the variety of sequence comparison algorithms and programs known in the art. For sequence comparison, typically one sequence acts as a reference sequence (e.g., a sequence disclosed herein), to which test sequences are compared. A sequence comparison algorithm then calculates the percent sequence identities for the test sequences relative to the reference sequence, based on the program parameters.

The percent identity of two amino acid or two nucleic acid sequences can be determined for example by comparing sequence information using the computer program GAP, i.e., Genetics Computer Group (GCG; Madison, Wis.) Wisconsin package version 10.0 program, GAP (Devereux et al. (1984), Nucleic Acids Res. 12: 387-95). In calculating percent identity, the sequences being compared are typically aligned in a way that gives the largest match between the sequences. The preferred default parameters for the GAP program include: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14: 6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps.

Sequence identity and/or similarity can also be determined by using the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (BESTFIT, FASTA, and TFASTA in the Wisconsin Genetics Software Package, Genetics Computer Group, 575 Science Drive, Madison, Wis.).

Another example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xᵤ set to 16, and X_{g} set to 40 for database search stage and to 67 for the output stage of the algorithms. Gapped alignments are triggered by a score corresponding to about 22 bits.

Methods for making the viral polypeptides described herein are well known to people of ordinary skill in the art. For example and without limitations, nucleic acid sequences encoding the viral polypeptides comprising SEQ ID NOs 7-11 or sequences at least 80% identical thereto, including conservatively substituted variants of SEQ ID NOs 7-11 may be cloned into a vector, such as, for example, a plasmid or a viral vector, and expressed in a suitable host such as fish cells, mammalian cells, bacterial cells, plant cells, and insect cells, and the expressed viral polypeptides separated therefrom.

### Viruses

Another aspect of the invention provides a virus that infects and is capable of killing lumpsucker fish (Cyclopterus lumpus), wherein the virus genome comprises a nucleic acid sequence disclosed herein, wherein said nucleic acid sequence contains the base uracil (U) instead of the base thymine (T).

Another aspect of the invention provides a virus that infects and is capable of killing lumpsucker fish (Cyclopterus lumpus), wherein the virus comprises a viral polypeptide comprising an amino acid sequence that is at least 80%, at least 90%, or at least 95% identical to any one of SEQ ID NOs 7-11, or that is any one of SEQ ID NOs 7-11 or a conservatively substituted variant thereof.

Another aspect of the invention provides a virus that infects and is capable of killing lumpsucker fish (Cyclopterus lumpus), wherein the virus genome comprises a nucleic acid sequence disclosed herein, wherein said nucleic acid sequence contains the base uracil (U) instead of the base thymine (T), and wherein the virus comprises a viral polypeptide comprising an amino acid sequence that is at least 80%, at least 90%, or at least 95% identical to any one of SEQ ID NOs 7-11, or that is any one of SEQ ID NOs 7-11 or a conservatively substituted variant thereof.

In preferred embodiments, the nucleic acid comprised within the virus is in the form of single-stranded RNA (ssRNA).

In some embodiments, the virus genome comprises a nucleic acid sequence comprising at least one of ORF-1, ORF-2, ORF-3, ORF-4 or ORF-5, according to any of their embodiments disclosed herein. In preferred embodiments, the sequence of the virus genome comprises at least ORF-1, ORF-2, ORF-3 and ORF-4, according to any of their embodiments disclosed herein. In some embodiments, the virus genome comprises a nucleic acid sequence which is, or which is complementary to, a nucleic acid sequence which is at least 80%, preferably at least 85%, more preferably at least 90%, even more preferably at least 95%, yet even more preferably 98%, particularly preferably 99%, or even 100% identical to the sequence of the virus genome according to SEQ ID NO:6 (CLuCV).

In some embodiments, the virus comprises ORF-1, ORF-2, ORF-3, ORF-4 and ORF-5; wherein
ORF-1 is at least 80% identical to the nucleic acid sequence of SEQ ID NO:1,
ORF-2 is at least 80% identical to the nucleic acid sequence of SEQ ID NO:2,
ORF-3 is at least 80% identical to the nucleic acid sequence of SEQ ID NO:3,
ORF-4 is at least 80% identical to the nucleic acid sequence of SEQ ID NO:4, and
ORF-5 is at least 80% identical to the nucleic acid sequence of SEQ ID NO:5;
   and wherein ORF-1, ORF-2, ORF-3, ORF-4 and ORF-5 encode viral polypeptides comprising SEQ ID NOs 7-11, respectively, or sequences that are at least 80% identical to SEQ ID NOs 7-11, respectively.

In some embodiments, ORF-1, ORF-2, ORF-3, ORF-4 or ORF-5 encode viral polypeptides comprising respective amino acid sequences comprising SEQ ID NOs 7-11 or sequences at least 80% identical thereto (for example, at least 85%, at least 90%, at least 91%, at least 92%, at least 93%, at least 94%, at least 95%, at least 96%, at least 97%, at least 98%, or at least 99%, identical to SEQ ID NOs 7-11). Preferably, the virus comprises ORF-1, ORF-2, ORF-3, ORF-4 and ORF-5 as defined herein, wherein said ORF-1, ORF-2, ORF-3, ORF-4 and ORF-5 encode viral polypeptides that are at least 95% identical to SEQ ID NOs 7-11, respectively. More preferably, the virus comprises ORF-1, ORF-2, ORF-3, ORF-4 and ORF-5 as defined herein, wherein said ORF-1, ORF-2, ORF-3, ORF-4 and ORF-5 encode viral polypeptides that are conservatively substituted variants of SEQ ID NOs 7-11, respectively, or viral polypeptides comprising the amino acid sequences of SEQ ID NOs 7-11, respectively.

In some embodiments, the infection of lumpsucker fish by the virus disclosed herein causes the following symptoms in the fish:
(i) tissue damage in the intestine, and/or
(ii) diarrhoea.

In further embodiments, the infection of lumpsucker fish by the virus disclosed herein additionally causes anorexia.

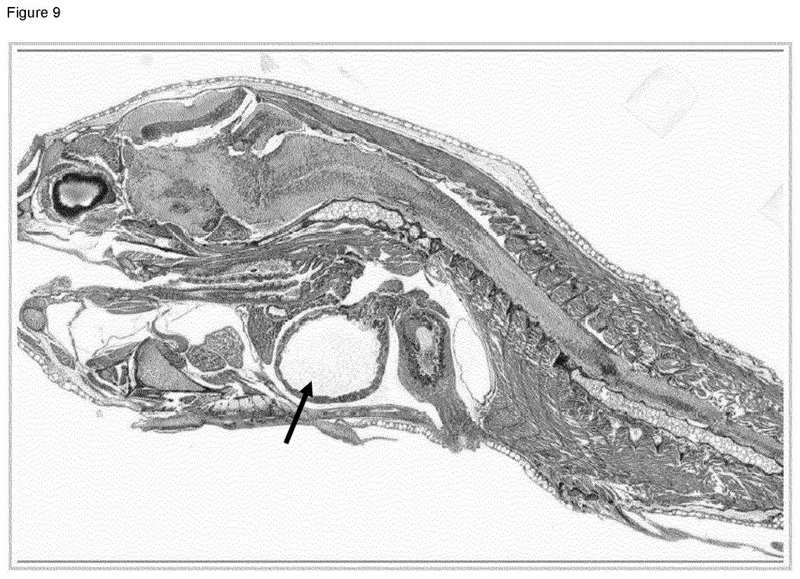

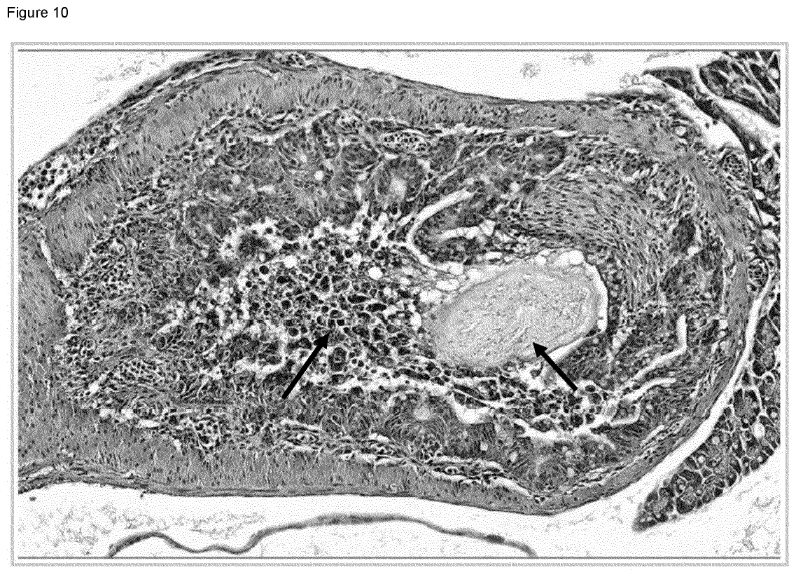

Tissue damage in the intestine can be diagnosed by the use of histology or electron microscopy to observe destruction of intestinal tissue, for example, destruction of the structure of villi, increased thickness of other layers. Preferably, tissue sections are stained using a Haematoxylin and Eosin (H&E) histological stain. In employing such a stain, infected individuals can be observed with accumulation of fluids and undigested feed particles in both the stomach and the intestine (see, e.g., FIGS. 9 to 12), leading to a diarrhoea-like condition in these fish. In addition, damage to the intestinal wall, with cellular discharge and increased mucus production can be observed (see, e.g., FIGS. 10 to 12). Fish diarrhoea can be observed in the water tanks of the farmed fish.

Changes in bio-macromolecule components (proteins in general, siderophile proteins, neutral mucopolysaccharides, glycogen and acid mucopolysaccharides) can also be observed in intestinal tissue samples by conventional methods, e.g., Western blotting, ELISA, staining of tissue sections, etc.

Anorexia in fish can be observed by the fish not feeding.

In some embodiments, the virus is an enveloped virus. Enveloped viruses have a protective layer (envelope) covering their protein capsids. The envelopes are typically derived from portions of the host cell membranes (phospholipids and proteins), but include some viral glycoproteins. They may help viruses avoid the host immune system.

In some embodiments, the virus is a Coronavirus. In preferred embodiments, the virus is a Torovirus. Such viruses are enveloped, round-shaped but pleomorphic, and around 100 to 150 nm in diameter. The virus particle typically has surface spikes proteins that are club-shaped and are evenly dispersed over the surface.

In some embodiments, the virus disclosed herein comprises a 5′ untranslated region (5′ UTR) which functions as an internal ribosome entry site (IRES).

In preferred embodiments, ORF-1 codes for a polyprotein, ORF-2 codes for a spike glycoprotein, ORF-3 codes for a membrane protein and ORF-4 codes for a nucleocapsid protein.

Also provided herein is a vector comprising a nucleic acid that encodes at least one ORF, as disclosed herein with all their embodiments. In some embodiments, the vector comprises a nucleic acid that encodes the complete virus disclosed herein. The vector can be used to introduce said nucleic acid(s) into a cell, such as a host cell.

Also provided herein is a host cell comprising the virus disclosed herein. The host cell may be a bacterial cell, a fish cell or a mammalian cell.

### Oligonucleotide Primers

Another aspect of the invention provides an oligonucleotide primer which comprises a sequence of at least 9 nucleotides, wherein said sequence is complementary to a nucleic acid sequence which is comprised within the genome of the virus disclosed herein.

Another aspect of the invention provides an oligonucleotide primer which comprises (a) a sequence of at least 9 consecutive nucleotides, wherein said sequence is complementary to a nucleic acid sequence which is comprised within the genome of the virus disclosed herein, (b) comprises at least 9 consecutive nucleotides of a sequence which is, or which is complementary to, a portion of a reference nucleic acid sequence selected from the group consisting of SEQ ID NO:1, SEQ ID NO:2, SEQ ID NO:3, SEQ ID NO:4, SEQ ID NO:5 or SEQ ID NO:6, or (c) comprises at least 9 consecutive nucleotides of a sequence which is at least 80% identical to a sequence which is, or which is complementary to, a sequence selected from the group consisting of SEQ ID NO:12 to SEQ ID NO:80; preferably with the proviso that said oligonucleotide primer does not comprise a sequence selected from the group consisting of SEQ ID NO:21 to SEQ ID NO:23.

In some embodiments, the oligonucleotide primer is 9 to 60 nucleotides in length. In preferred embodiments, the oligonucleotide primer is 12 to 40 nucleotides in length. In more preferred embodiments, the oligonucleotide primer is 15 to 30 nucleotides in length. In even more preferred embodiments, the oligonucleotide primer is 18 to 25 nucleotides in length.

As the skilled person will readily appreciate, an oligonucleotide primer which is complementary to a nucleic acid sequence will hybridize to that sequence under stringent conditions. The “stringent conditions” refer to conditions of temperature, ionic strength, and the presence of other compounds such as organic solvents, under which nucleic acid hybridizations are conducted. Under stringent conditions, nucleic acid base pairing will occur only between nucleic acid seqeunces having a high frequency of complementary bases. Stringent hybridization conditions are known to the skilled person (see e.g. Green M. R., Sambrook, J., Molecular Cloning: A Laboratory Manual, Cold Spring Harbor Laboratory Press; 4th edition, 2012). The precise conditions for stringent hybridization are typically sequence-dependent and will be different in different circumstances, as the skilled person will readily appreciate. Longer sequences hybridize at higher temperatures compared to shorter sequences. Generally, stringent conditions are selected to be about 5° C. lower than the thermal melting point (Tm) for the specific sequence. The Tm is defined as the temperature when 50% of the duplex molecules have dissociated into their constituent single strands. Since the target sequences are generally present at excess, at Tm, 50% of nucleic acid primers would generally be occupied at equilibrium. Typically, stringent conditions will be those in which the salt concentration is less than about 1.0 M sodium ions, typically about 0.01 to 1.0 M sodium ions (or other salts) at pH 6.8 to 8.3 and the temperature is at least about 30° C. for short primers (e.g., 10 nucleotides to 50 nucleotides) and at least about 60° C. for longer primers. Stringent conditions may also be achieved with the addition of destabilizing agents, such as formamide. As the skilled person will readily appreciate, due to sequence complementarity of the sequences, the oligonucleotide primer according to the invention therefore hybridizes to a nucleic acid sequence which is comprised within the genome of the virus disclosed herein.

An oligonucleotide primer according to the present invention may be labelled with a molecular marker in order to enable visualization of hybridization to target sequence or quantification of amplification of target sequence. Various molecular markers or labels are known to the skilled person.

In a particular embodiment, herein provided is an oligonucleotide primer which comprises at least 9 consecutive nucleotides of a sequence which is, or which is complementary to, a portion (e.g., 9 to 60 nucleotides in length, preferably 12 to 40 nucleotides in length, more preferably 15 to 30 nucleotides in length, even more preferably 18 to 25 nucleotides in length) of a reference nucleic acid sequence selected from the group consisting of SEQ ID NO:1, SEQ ID NO:2, SEQ ID NO:3, SEQ ID NO:4, SEQ ID NO:5 or SEQ ID NO:6; preferably with the proviso that said oligonucleotide primer does not comprise a sequence selected from the group consisting of SEQ ID NO:21 to SEQ ID NO:23. In a particularly preferred embodiment, the sequence of said oligonucleotide primer consists of said sequence of consecutive nucleotides.

In another particular embodiment, herein provided is an oligonucleotide primer which comprises at least 9 (preferably at least 12, more preferably at least 15, even more preferably at least 18) consecutive nucleotides of a sequence which is at least 80% identical to a sequence which is, or which is complementary to, a sequence selected from the group consisting of SEQ ID NO:12 to SEQ ID NO:80;

preferably with the proviso that said oligonucleotide primer does not comprise a sequence selected from the group consisting of SEQ ID NO:21 to SEQ ID NO:23.

Another aspect of the invention provides a use of at least one oligonucleotide primer in a method of detecting the virus disclosed herein, wherein the at least one primer comprises a sequence of at least 9 nucleotides, e.g., 9 consecutive nucleotides, (preferably at least 12, more preferably at least 15, even more preferably at least 18) and wherein said sequence is complementary to a nucleic acid sequence which is comprised within the genome of said virus.

In some embodiments, the at least one oligonucleotide primer is a primer pair, i.e., two primers, one forward primer and one reverse primer, which are complementary to two regions on a nucleic acid sequence, and which can be used to amplify a sequence between said two regions. This is well known to a skilled person, and it is within his/her skills to find oligonucleotide primers suitable to constitute a pair. According to the use of the invention, a “primer pair” can be used to amplify a nucleic acid sequence which is comprised within the genome of the virus disclosed herein.

In some embodiments, the use employs cDNA synthesis in a method of detecting the virus disclosed herein. For example, random oligonucleotide primers (e.g., hexanucleotides) are used to synthesize cDNAs from a nucleic acid sequence which is comprised within the genome of said virus.

In some embodiments, the use employs PCR in a method of detecting the virus disclosed herein. In some embodiments, the use employs RT-PCR in a method of detecting the virus disclosed herein. In some embodiments, the use employs RT-qPCR in a method of detecting the virus disclosed herein. In some embodiments, the use employs Random Multiplex RT-PCR in a method of detecting the virus disclosed herein; this method uses a mixture of primers designed to be resistant to primer-dimer amplification (see Clem et al, Virol J, 4, 2007). In some embodiments, the use employs transcription mediated amplification (TMA) in a method of detecting the virus disclosed herein. In some embodiments, the use employs strand displacement amplification (SDA) in a method of detecting the virus disclosed herein.

In some embodiments, the use employs in situ detection, also termed in situ hybridization (ISH) in a method of detecting the virus disclosed herein, for example fluorescence in situ hybridization (FISH). ISH uses a labelled complementary DNA, RNA or modified oligonucleotide primer sequence (probe) to enable visualization of specific nucleic acids in morphologically preserved cells and tissue sections. The probe can be labelled with radio-, fluorescent- or antigen-labels (e.g., digoxigenin), which can then be localized and quantified in the tissue using either autoradiography, fluorescence microscopy, or immunohistochemistry, respectively.

### Diagnostic Methods

Another aspect of the invention provides a method for detecting a virus that infects and is capable of killing fish, comprising the steps of:
(a) contacting a nucleic acid extracted from a biological sample of a fish with at least one oligonucleotide primer to form a mixture, wherein the at least one oligonucleotide primer is complementary to a nucleic acid sequence which is comprised within the genome of the virus disclosed herein, and
(b) determining whether upon subjecting the mixture of a) to amplification an amplification product is present, wherein the presence of amplification product indicates the presence of RNA associated with the virus, and hence the presence of the virus in the biological sample.

In some embodiments, nucleic acid in step (a) of the method, for example RNA, is extracted from biological samples by using solid-phase extraction, e.g., on-column purification using a solid phase of silica gel membrane. In some embodiments, nucleic acid in step (a) of the method, for example RNA, is extracted from biological samples by using phenol/chloroform extraction.

The method may use any suitable oligonucleotide primer disclosed herein. Generally, the at least one oligonucleotide primer of step (a) of the method is chosen to produce an amplification product according to step (b) which has a length of 45 nucleotides to 3000 nucleotides. However, amplification products even smaller or greater in length may suitably be produced by the methods and oligonucleotide primer disclosed herein.

In some embodiments, the at least one oligonucleotide primer of step (a) of the method is chosen to produce an amplification product according to step (b) for a PCR or RT-PCR assay which has a length of 100 nucleotides to 2500 nucleotides. In preferred embodiments, the at least one oligonucleotide primer of step (a) of the method is chosen to produce an amplification product according to step (b) for a PCR or RT-PCR assay which has a length of 200 nucleotides to 1500 nucleotides. In more preferred embodiments, the at least one oligonucleotide primer of step (a) of the method is chosen to produce an amplification product according to step (b) for a PCR or RT-PCR assay which has a length of 300 nucleotides to 1000 nucleotides.

In some embodiments, the at least one oligonucleotide primer of step (a) of the method is chosen to produce an amplification product according to step (b) for a real-time RT-PCR assay which has a length of 45 nucleotides to 500 nucleotides. In preferred embodiments, the at least one oligonucleotide primer of step (a) of the method is chosen to produce an amplification product according to step (b) for a real-time RT-PCR assay which has a length of 50 nucleotides to 350 nucleotides. In more preferred embodiments, the at least one oligonucleotide primer of step (a) of the method is chosen to produce an amplification product according to step (b) for a real-time RT-PCR assay which has a length of 55 nucleotides to 250 nucleotides.

In some embodiments, the amplification of step (b) of the method employs PCR. In some embodiments, the amplification of step (b) of the method employs RT-PCR. In some embodiments, the amplification of step (b) of the method employs RT-qPCR.

In some embodiments, the amplification product of step (b) of the method is determined by Southern blot. In some embodiments, the amplification product of step (b) of the method is determined by Northern blot. In some embodiments, the amplification product of step (b) of the method is determined by spectrophotometry. In some embodiments, the amplification product of step (b) of the method is determined by use of a DNA dye. In some embodiments, the amplification product of step (b) of the method is determined by quantifying the presence of a labelled oligonucleotide primer, for example, quantifying the presence of a fluorescently-labelled oligonucleotide primer.

Another aspect of the invention provides a method for detecting a virus that infects and is capable of killing fish, comprising the steps of:
(a) sequencing a nucleic acid extracted from a biological sample of a fish, and
(b) comparing the resulting nucleic acid sequence with a nucleic acid sequence which is, or which is complementary to, a reference sequence selected from the group consisting of SEQ ID NO:1, SEQ ID NO:2, SEQ ID NO:3, SEQ ID NO:4, SEQ ID NO:5 and SEQ ID NO:6, wherein an at least 80% sequence identity between the two sequences indicates the presence of the virus in the biological sample.

In some embodiments, nucleic acid in step (a) of the method, for example RNA, is extracted from biological samples by using solid-phase extraction, e.g., on-column purification using a solid phase of silica gel membrane. In some embodiments, nucleic acid in step (a) of the method, for example RNA, is extracted from biological samples by using phenol/chloroform extraction.

In preferred embodiments, the sequencing in step (a) of the method provides a DNA sequence, which can be directly compared to the reference DNA sequences selected from the group consisting of SEQ ID NO:1, SEQ ID NO:2, SEQ ID NO:3, SEQ ID NO:4, SEQ ID NO:5 and SEQ ID NO:6.

In some embodiments, the sequencing in step (a) of the method is performed by Sanger sequencing (chain termination method). In preferred embodiments, the sequencing in step (a) of the method is performed by Next Generation Sequencing (NGS), preferably Illumina (Solexa) sequencing, Roche 454 sequencing, Ion Torrent or SOLiD sequencing (Goodwin S, et al., (2016) Coming of age: Ten years of next-generation sequencing technologies. Nature reviews, Genetics, 17, 333-351).

In some embodiments, an at least 85% sequence identity between the two sequences in step (b) of the method indicates the presence of the virus in the biological sample.

In preferred embodiments, an at least 90% sequence identity between the two sequences in step (b) of the method indicates the presence of the virus in the biological sample.

In more preferred embodiments, an at least 95% sequence identity between the two sequences in step (b) of the method indicates the presence of the virus in the biological sample.

In yet more preferred embodiments, an at least 98% sequence identity between the two sequences in step (b) of the method indicates the presence of the virus in the biological sample.

In yet even more preferred embodiments, an at least 99% sequence identity between the two sequences in step (b) of the method indicates the presence of the virus in the biological sample.

In a particularly preferred embodiment, a 100% sequence identity between the two sequences in step (b) of the method indicates the presence of the virus in the biological sample.

Another aspect of the invention provides a method for detecting a virus that infects and is capable of killing fish, comprising the steps of:
(a) sequencing a nucleic acid extracted from a biological sample of a fish, and
(b) translating the resulting nucleic acid sequence into an amino acid sequence or translating a nucleic acid sequence complementary to said resulting nucleic acid sequence into an amino acid sequence, and
(c) comparing the resulting amino acid sequence with a reference sequence selected from the group consisting of SEQ ID NOs 7-11, wherein an at least 80% sequence identity between the two sequences indicates the presence of the virus in the biological sample.

### Antibodies

Another aspect of the invention provides an antibody that binds a polypeptide, wherein the polypeptide is encoded by a nucleic acid sequence which is comprised within the genome of the virus disclosed herein, and/or wherein the polypeptide comprises an amino acid sequence that is at least 80%, at least 90%, or at least 95% identical to any one of SEQ ID NOs 7-11, or that is any one of SEQ ID NOs 7-11 or a conservatively substituted variant thereof.

In some embodiments, the polypeptide is selected from the group consisting of:
(i) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:7;
(ii) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:8;
(iii) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:9;
(iv) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:10; and
(v) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:11.

In some embodiments, the antibody is a polyclonal antibody. In some embodiments, the antibody is a monoclonal antibody.

The antibodies disclosed herein may be prepared by genetic immunization methods in which native proteins are expressed in vivo with normal post-transcriptional modifications, avoiding antigen isolation or synthesis. For example, hydrodynamic tail or limb vein delivery of naked plasmid DNA expression vectors can be used to produce the antigen of interest in vivo in mice, rats, and rabbits and thereby induce antigen-specific antibodies (Tang et al, Nature 356(6365): 152-4 (1992); Tighe et al, Immunol. Today 19(2) 89-97 (1998); Bates et al, Biotechniques, 40 (2) 199-208 (2006)). This allows the efficient generation of high-titre, antigen-specific antibodies. Antibodies can also be derived by in vitro methods. Suitable examples include but are not limited to hybridoma technologies, phage display, yeast display and the like.

### Kits

Another aspect of the invention provides a kit for detecting a virus in a biological sample from fish, wherein the kit comprises an oligonucleotide primer disclosed herein and/or an antibody disclosed herein.

In some embodiments, the kit is a real-time RT-PCR assay, for example the kit is a real-time RT-qPCR assay.

In some embodiments, the kit is for detecting a Coronavirus in a biological sample from fish. In preferred embodiments, the kit is for detecting a Torovirus in a biological sample from fish. In more preferred embodiments, the kit is for detecting a Torovirus in a biological sample from lumpsucker fish.

### Medical Uses and Vaccines

Another aspect of the invention provides an antibody for use in treating fish, particularly lumpsucker fish, against disease caused by Coronavirus, specifically Torovirus, infection. In a preferred embodiment, the antibody is for use in treating fish, particularly lumpsucker fish, against disease caused by the virus disclosed herein (CLuCV). The antibody binds a polypeptide which is encoded by a nucleic acid sequence comprised within the genome of the virus disclosed herein.

In preferred embodiments, the fish is a lumpsucker fish.

In some embodiments, the fish show the following symptoms:
(i) tissue damage in the intestine, and/or
(ii) diarrhoea.

In further embodiments, the fish additionally show symptoms of anorexia.

The symptoms can be determined as described above.

Another aspect of the invention provides a use of virus disclosed herein for producing a vaccine against disease caused by said virus.

Another aspect of the invention provides a vaccine comprising:
(i) a nucleic acid sequence which is comprised within the genome of the virus disclosed herein;
(ii) a nucleic acid sequence disclosed herein;
(iii) a viral polypeptide encoded by a nucleic acid sequence which is comprised within the genome of the virus disclosed herein;
(iv) a viral polypeptide comprising an amino acid sequence that is at least 80%, at least 90%, or at least 95% identical to any one of SEQ ID NOs 7-11, or that is any one of SEQ ID NOs 7-11 or a conservatively substituted variant thereof; or
(v) a virus disclosed herein.

In certain embodiments, the vaccine contains several viral polypeptides, e.g., a first polypeptide that comprises an amino acid sequence that is at least 80% identical to SEQ ID NO: 7 and a second polypeptide that comprises an amino acid sequence that is at least 80% identical to SEQ ID NO: 8. The vaccine may contain one, two, three, four, or five viral polypeptides.

The vaccine is for protecting fish, particularly lumpsucker fish, against disease caused by Coronavirus, specifically Torovirus, infection. In a preferred embodiment, the vaccine is for protecting fish, particularly lumpsucker fish, against disease caused by infection with the virus disclosed herein (CLuCV).

In some embodiments where the vaccine comprises a nucleic acid sequence comprised within the genome of the virus disclosed herein, said nucleic acid sequence comprises at least one of ORF-1, ORF-2, ORF-3, ORF-4 or ORF-5, according to any of their embodiments disclosed herein. In preferred embodiments where the vaccine comprises a nucleic acid sequence comprised within the genome of the virus disclosed herein, said nucleic acid sequence comprises at least ORF-1, ORF-2, ORF-3 and ORF-4, according to any of their embodiments disclosed herein. In some embodiments where the vaccine comprises a nucleic acid sequence comprised within the genome of the virus disclosed herein, said nucleic acid sequence is, or is complementary to, a nucleic acid sequence which is at least 80%, preferably at least 85%, more preferably at least 90%, yet more preferably at least 95%, even more preferably at least 98%, particularly preferably at least 99%, or even 100% identical to, the sequence of the virus genome according to SEQ ID NO:6 (CLuCV).

The nucleic acid may be DNA or RNA.

In some embodiments where the vaccine comprises a viral polypeptide encoded by a nucleic acid sequence which is comprised within the genome of the virus disclosed herein, said polypeptide is selected from the group consisting of:
(i) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:7;
(ii) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:8;
(iii) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:9;
(iv) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:10; and
(v) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, or which is, SEQ ID NO:11.

In some embodiments where the vaccine comprises a virus disclosed herein, the genome of said virus comprises a nucleic acid sequence which is an RNA nucleic acid sequence comprising at least one of ORF-1, ORF-2, ORF-3, ORF-4 or ORF-5, according to any of their embodiments disclosed herein. In some embodiments where the vaccine comprises a virus disclosed herein, the genome of said virus comprises a nucleic acid sequence which is an RNA nucleic acid sequence which is, or which is complementary to, a nucleic acid sequence which is at least 80%, preferably at least 85%, more preferably at least 90%, even more preferably at least 95%, yet even more preferably 98%, particularly preferably 99%, or even 100% identical to the sequence of the virus genome according to SEQ ID NO:6 (CLuCV).

In some embodiments, the vaccine comprises an amount of antigen which is in the range of 0.05 to 1.0 mg/ml, such as from 0.1 to 0.5 mg/ml, from 0.15 to 0.4 mg/ml, or from 0.2 to 0.3 mg/ml. The vaccine may be for administration in dosages of 0.005 to 0.5 mg/individual, preferably from 0.01 to 0.05 mg/individual, more preferably from 0.01 to 0.02 mg/individual.

In some embodiments, the vaccine comprises an amount of antigen corresponding to a TCID50 of 10⁵ to 10¹⁰ per dosage, preferably a TCID₅₀ of 10⁶ to 10⁹ per dosage.

The vaccine may be in the form of a suspension of the virus or it may be lyophilized. In a lyophilized vaccine it may be useful to add one or more stabilizers. Suitable stabilizers are for example carbohydrates such as sorbitol, mannitol, starch, sucrose, dextran; protein containing agents such as bovine serum or skimmed milk; and buffers such as alkali metal phosphates.

The vaccine according to the invention may further be in a formulation comprising an adjuvant. Examples of adjuvants frequently used in fish and shellfish farming are muramyldipeptides, lipopolysaccharides, several glucans and glycans, mineral oil, Montanide™ and Carbopol®. An overview of adjuvants suitable for fish vaccines is given in the review paper of Sommerset (Expert Rev. Vaccines 4 (1), 89-101 (2005)).

The vaccine of the invention may further comprise a suitable pharmaceutical carrier. In some embodiments the vaccine is formulated as an emulsion of water in oil. The vaccine may also comprise a so-called “vehicle”. A vehicle is a device to which the antigen adheres, without being covalently bound to it. Such vehicles are inter alia biodegradable nano/micro-particles or -capsules of PLGA (poly-lactide-co-glycolic acid), alginate or chitosan, liposomes, niosomes, micelles, multiple emulsions and macrosols, all known in the art. A special form of such a vehicle, in which the antigen is partially embedded in the vehicle, is the so-called ISCOM.

In addition, the vaccine may comprise one or more suitable surface-active compounds or emulsifiers, e.g., Cremophore®, Tween® and Span®. Also, adjuvants such as interleukin, CpG and glycoproteins may be used.

In some embodiments, the vaccine is provided in fish feed, said feed may for example be pelleted or extruded feed.

Another aspect of the invention provides an interfering RNA (iRNA) molecule for use in treating fish, particularly lumpsucker fish, against disease caused by Coronavirus, specifically Torovirus, infection. In a preferred embodiment, the iRNA is for use in treating fish, particularly lumpsucker fish, against disease caused by the virus disclosed herein (CLuCV). In a particular embodiment, the iRNA molecule comprises at least 12 (preferably contiguous) nucleotides of, or complementary to, a nucleic acid sequence comprised within the genome of the virus disclosed herein.

In preferred embodiments, the fish is a lumpsucker fish.

In some embodiments, the fish show the following symptoms:
(i) tissue damage in the intestine, and/or
(ii) diarrhoea.

In further embodiments, the fish additionally show symptoms of anorexia.

The symptoms can be determined as described above.

In some embodiments, the iRNA molecule comprises at least 12 (preferably contiguous) nucleotides of, or complimentary to, a nucleic acid sequence comprising ORF-1, ORF-2, ORF-3, ORF-4 or ORF-5, according to any of their embodiments disclosed herein. In some embodiments, the iRNA molecule comprises at least 12 (preferably contiguous) nucleotides of, or complimentary to, a nucleic acid sequence which is at least 80%, preferably at least 85%, more preferably at least 90%, even more preferably at least 95%, yet even more preferably 98%, particularly preferably 99%, or even 100% identical to the sequence of the virus genome according to SEQ ID NO:6 (CLuCV).

### Sequences

The sequences referred to in the present disclosure are the following (see also the Figures and the sequence listing):

TABLE 1

| Sequence | Description |
|---|---|
| SEQ ID NO: 1 | CLuCV_ORF-1 nucleotide sequence (Polyprotein) |
| SEQ ID NO: 2 | CLuCV_ORF-2 nucleotide sequence (Spike |
| | Glycoprotein) |
| SEQ ID NO: 3 | CLuCV_ORF-3 nucleotide sequence (Membrane |
| | protein) |
| SEQ ID NO: 4 | CLuCV_ORF-4 nucleotide sequence (Nucleocapsid) |
| SEQ ID NO: 5 | CLuCV_ORF-5 nucleotide sequence (Unknown) |
| SEQ ID NO: 6 | CLuCV_Genome nucleotide sequence |
| SEQ ID NO: 7 | CLuCV_ORF-1 amino acid sequence (Polyprotein) |
| SEQ ID NO: 8 | CLuCV_ORF-2 amino acid sequence (Spike |
| | Glycoprotein) |
| SEQ ID NO: 9 | CLuCV_ORF-3 amino acid sequence (Membrane |
| | protein) |
| SEQ ID NO: 10 | CLuCV_ORF-4 amino acid sequence (Nucleocapsid) |
| SEQ ID NO: 11 | CLuCV_ORF-5 amino acid sequence (Unknown) |
| SEQ ID NO: 12 | TCL-ORF-1 Forward primer |
| SEQ ID NO: 13 | TCL-ORF-1 Taqman Probe |
| SEQ ID NO: 14 | TCL-ORF-1 Reverse primer |
| SEQ ID NO: 15 | TCL-M Forward primer |
| SEQ ID NO: 16 | TCL-M Taqman Probe |
| SEQ ID NO: 17 | TCL-M Reverse primer |
| SEQ ID NO: 18 | TCL-S Forward primer |
| SEQ ID NO: 19 | TCL-S Taqman Probe |
| SEQ ID NO: 20 | TCL-S Reverse primer |

Additional useful primers referred to herein are provided in Table 2 below:

| SEQ ID | Primer | |
|---|---|---|
| NO: | name | primer sequence |
| 24 | TCL_F1 | CAGTCCACCAACACAACGTG |
| 25 | TCL_R1 | CCCAAGTGTCGCTTTGCATC |
| 26 | TCL_F2 | CCAACAAAGGAGCCGCAATC |
| 27 | TCL_R2 | GCACATGTTTGGTGGGTGTC |
| 28 | TCL_F3 | GACACCCACCAAACATGTGC |
| 29 | TCL_R3 | GCCAAACGGAGGTCTGGATT |
| 30 | TCL_F4 | AATCCAGACCTCCGTTTGGC |
| 31 | TCL_R4 | TCAATACCTCGAGCGCAGAC |
| 32 | TCL_F5 | TGTCTGCGCTCGAGGTATTG |
| 33 | TCL_R5 | TTTGTTCAGGGGTGGTGTCC |
| 34 | TCL_F6 | TCTGACGACAAACCGGACAC |
| 35 | TCL_R6 | AGCCGCTGGGATTACTTCAC |
| 36 | TCL_F7 | TTCCTAGCAACCCGCTTTCA |
| 37 | TCL_R7 | AGCGGTTTTCTTTTCCGTCG |
| 38 | TCL_F8 | CGACGGAAAAGAAAACCGCT |
| 39 | TCL_R8 | ACTACGGCTACTGGGGTTC |
| 40 | TCL_F9 | CCCCAGTAGCCGTAGTTGAC |
| 41 | TCL_R9 | TCATGCCGGAGATTTTGCCT |
| 42 | TCL_F10 | AGGCAAAATCTCCGGCATGA |
| 43 | TCL_R10 | AGATGAGCCGTCAGCAAACA |
| 44 | TCL_F11 | TGTTTGCTGACGGCTCATCT |
| 45 | TCL_R11 | TGCGTTTGCTCTGTCGTAGT |
| 46 | TCL_F12 | ACTACGACAGAGCAAACGCA |
| 47 | TCL_R12 | ATAGAGCGCAAGCCGTAGAC |
| 48 | TCL_F13 | GTCTACGGCTTGCGCTCTAT |
| 49 | TCL_R13 | GCCAGTAACACCATGTCCCA |
| 50 | TCL_F14 | TGGGACATGGTGTTACTGGC |
| 51 | TCL_R14 | AACGCTGTTACGCGGTTTTC |
| 52 | TCL_F15 | GAAAACCGCGTAACAGCGTT |
| 53 | TCL_R15 | CTGGCATCGTGTTGTGTGTG |
| 54 | TCL_F16 | CACACACAACACGATGCCAG |
| 55 | TCL_R16 | TAGACCCGCACTACCACACT |
| 56 | TCL_F17 | AGTGTGGTAGTGCGGGTCTA |
| 57 | TCL_R17 | TTTTATGACCACGTCCGCCA |
| 58 | TCL_F18 | TGGCGGACGTGGTCATAAAA |
| 59 | TCL_R18 | CTGCAGCTCCTCTGGTTTCA |
| 60 | TCL_F19 | TGAAACCAGAGGAGCTGCAG |
| 61 | TCL_R19 | GCCGAAGCTATTGTGAGGGT |
| 62 | TCL_F20 | ACCCTCACAATAGCTTCGGC |
| 63 | TCL_R20 | CGGAAAAACAACAGCCGAGG |
| 64 | TCL_F21 | CCTCGGCTGTTGTTTTTCCG |
| 65 | TCL_R21 | TGGGTCAGTAGGTGCGAGTA |
| 66 | TCL_F22 | TACTCGCACCTACTGACCCA |
| 67 | TCL_R22 | GGAGCCAACCGAGGATAAGG |
| 68 | TCL_F23 | TGGCATGGGTTCGTCCTTTT |
| 69 | TCL_R23 | GGTACCTTTCGCAATGACGC |
| 70 | TCL_F24 | GCGTCATTGCGAAAGGTACC |
| 71 | TCL_R24 | ACCCTCCGCCATTGTTGAAT |
| 72 | TCL_F25 | ATTCCAACCATCGCGGTTCT |
| 73 | TCL_R25 | TTCGCCTTGATACCAGCGTT |
| 74 | TCL_F26 | ACGCTGGTATCAAGGCGAAA |
| 75 | TCL_R26 | ATCCAGGAGTTAACGTCGGC |
| 76 | TCL_F27 | GCCGACGTTAACTCCTGGAT |
| 77 | TCL_R27 | AGGTCAGAACAAGGGAGGCT |
| | | A |
| 78 | Assay 1 | CATCTACCTCTCCCATACTC |
| | membrane | |
| | primer 1 | |
| 79 | Assay 1 | ACTGCTTCCAAAACTGATTA |
| | membrane | CCT |
| | primer 2 | |
| 80 | Assay 1 | GGGCGTAAAGAGAATGTAAG |
| | membrane3 | |
| | primer | |

Further oligonucleotide primers referred to herein are provided in Table 3:

| SEQ ID | |
|---|---|
| NO | primer sequence |
| 21 | TAATTTGACTGACTATAG |
| 22 | TAAGAAACTATACCAGTCCATGTCG |
| 23 | AGTTTAAGTGAG |

### EXAMPLES

The following examples illustrate the present invention. They are intended to aid in the understanding of the invention, and they should not be construed to in any way limit the scope of the invention.

### Example 1

### Identification of CLuCV

Fish samples were obtained from a lumpsucker (Cyclopterus lumpus) farming site experiencing high mortality (60-80%). The samples were analyzed using standard real-time RT-PCR and histology. No known pathogens were identified using real-time RT-PCR. Histological analysis indicated signs of tissue damage in the intestine in the affected individuals, but no potentially pathogenic bacteria and no microparasites could be observed.

Total RNA was extracted from moribund fish in a phenol/chloroform extraction protocol (Qiagen RNeasy® 96 Universal Tissue Kit), and this RNA was used as a template in a Next Generation Sequencing (NGS) analysis. The NGS analysis was performed by the company BaseClear (https://www.baseclear.com/), and yielded approximately 98 million sequence reads, where the majority came from the host transcriptome. Approximately 28,600 of the assembled reads from the NGS analysis were found to have no significant nucleotide match in the National Center for Biotechnology Information (NCBI) GenBank. From these reads, a novel virus sequence—herein termed Cyclopterus lumpus Coronavirus or CLuCV—0 was identified and characterized by the inventors. The DNA sequences obtained from the NGS analysis are provided in FIGS. 2 to 5 (see also SEQ ID Nos: 1 to 6). However, it will be appreciated that the corresponding RNA sequences are present in the virus CLuCV.

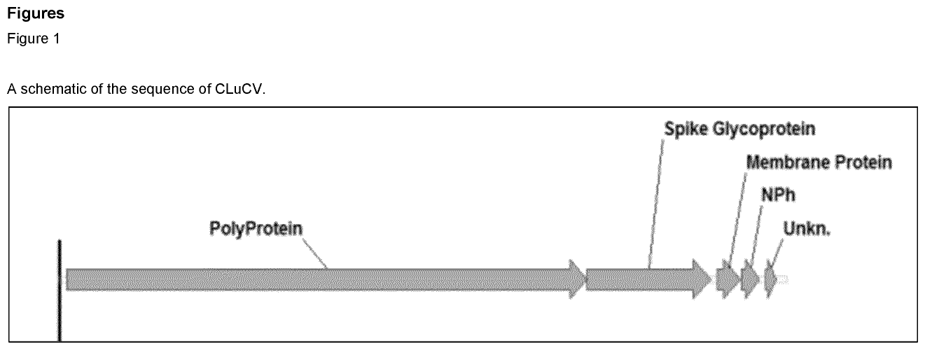

The sequence of CLuCV was identified to be 24,613 nucleotides long, and it contains five possible open reading frames (ORFs). A schematic is shown in FIG. 1. A relationship between CLuCV and other known viruses could only be determined by comparison of translated amino acid sequences. The length and organization of the genome, together with downstream sequence analyses, indicate that it is a novel Torovirus, in the Coronaviridae family, with the Berne virus (isolated from horses) as its closest relative.

Preliminary results indicate that, at least in Norway, CLuCV has a significant presence in farmed lumpsucker.

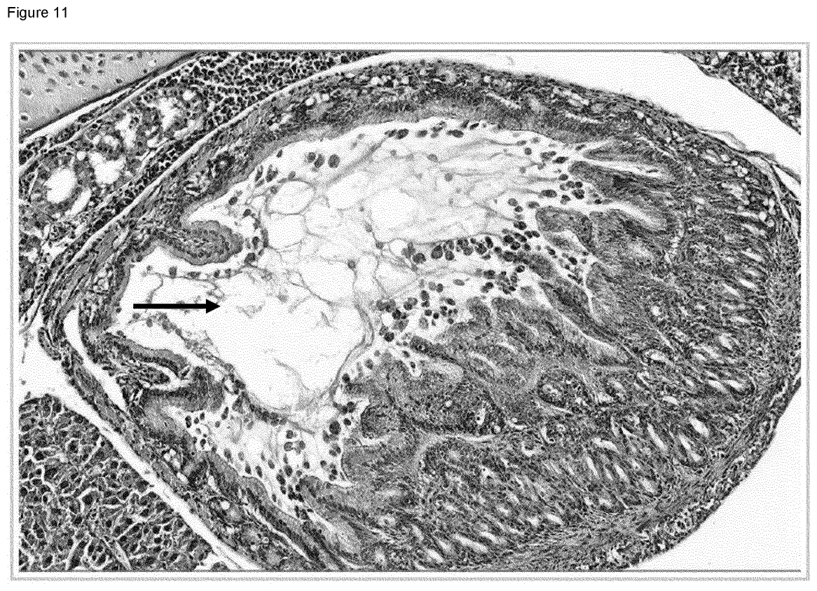

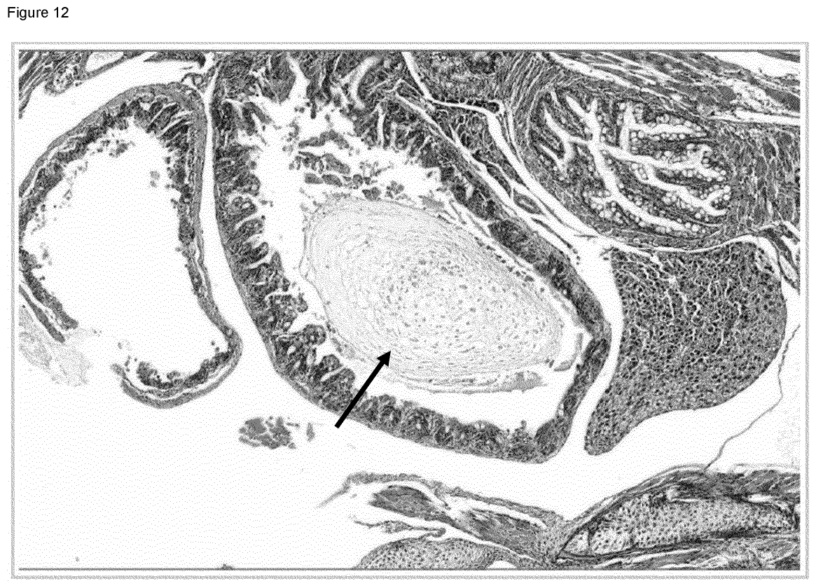

The pathology associated with CLuTV in lumpsucker is shown by FIGS. 9 to 12. Lumpsucker sections were stained using a Haematoxylin and Eosin (H&E) histological stain. FIG. 9 is a whole section of affected lumpsucker, showing accumulation of fluid in the stomach (arrow). FIG. 10 is a section of lumpsucker intestine showing accumulation of mucus, and cellular discharge (arrows). FIG. 11 is a section of lumpsucker intestine showing accumulation of mucus (arrow), and cellular discharge. FIG. 12 is a section of lumpsucker intestine showing accumulation of mucus (arrow).

### Example 2

### Nucleotide Sequence Analysis of CLuCV

When performing a standard nucleotide BLAST search using the CLuCV sequence, no match was found in the NCBI GeneBank. If parameters of the search were altered to include more dissimilar sequences in the search, some sequence regions from other known viruses were identified. The percentage of nucleotide sequence identity (“Seq. Id.”) between these CLuCV regions and regions from other known viruses is shown in Table 2.

TABLE 2
Best nucleotide match found between CLuCV and known viruses in the NCBI GeneBank

| | | Best sequence region match (NCBI GeneBank) | | | | | |
|---|---|---|---|---|---|---|---|
| CL_VirusB Region | | | | | Seq. | | |
| Start | Stop | Start | Stop | bp | Id. (%) | Acc. No. | Description |
| 11266 | 12315 | 15788 | 16837 | 1069 | 64% | MG996765 | Berne virus, isolate P138/72 |
| 11266 | 12315 | 2314 | 3363 | 1069 | 64% | X52374 | Berne virus mRNA, for RdRp |
| 11366 | 11893 | 15040 | 15931 | 535 | 67% | KM403390 | Porcine torovirus, strain PToV/NPL/2014 |
| 11366 | 11893 | 15400 | 15874 | 536 | 66% | JQ860350 | Porcine torovirus, strain SH1 |
| 11256 | 11849 | 15443 | 16036 | 601 | 65% | AY427798 | Breda virus strain Breda 1 |

### Example 3

### Amino Acid Sequence Analysis of CLuCV

Translation of the ORFs of CLuCV yields a total of five potential proteins. The amino acid identity between the CLuCV ORFs and other known proteins from other viruses is shown in Table 3. Only the best match for each ORF is provided.

TABLE 3
Best amino acid sequence match found between CLuCV and known viruses in the NCBI GeneBank

| CL_VirusB | | Best aa sequence match (NCBI GeneBank) | | | | | |
|---|---|---|---|---|---|---|---|
| | Amino | Total | Query | | Id. | | |
| ORF | acids | Score | cover. | E- value | (%) | Acc. No. | Description |
| ORF01 | 5867 | 2017 | 64% | 0.0 | 39% | AWV66923 | Polyprotein1ab, Berne virus |
| ORF02 | 1391 | 435 | 54% | 8.00E−125 | 34% | CAE01338 | Spike glycoprotein, Porcine Torovirus |
| ORF03 | 260 | 42.4 | 71% | 0.007 | 26% | YP337908 | Membrane glycoprotein, Breda virus |
| ORF04 | 201 | NA | NA | NA | NA | NA | No match |
| ORF05 | 113 | NA | NA | NA | NA | NA | No match |

ORF-1 corresponds to a polyprotein. ORF-2 corresponds to a spike glycoprotein. ORF-3 corresponds to a membrane glycoprotein. ORF-4 corresponds to a Nucleocapsid protein. ORF-5 corresponds to an unknown protein.

### Example 4

### Evaluating the Presence of CLuCV in Farmed Lumpfish Populations

Three separate real-time RT-PCR assays (TCL-ORF-1, TCL-M and TCL-S) were designed using the primers according to SEQ ID NOs: 12 to 14, 15 to 17 and 18 to 20, respectively. TCL-ORF-1 targeted ORF-1, TCL-M targeted ORF-3 (membrane protein), and TCL-S targeted ORF-2 (spike glycoprotein). The original fish sample material was confirmed to be positive for CLuCV using all three of these assays.

For more extensive investigation, assay TCL-ORF-1 was selected to evaluate the presence of CLuCV in existing farmed lumpfish populations. The results from screening different lumpfish populations in Norway are shown in Table 4.

TABLE 4
Results from testing of NGS material and field material
Screening of lumpfish populations (Norway)

| County | Samples (N) | Positive (N) | Prevalence (%) |
|---|---|---|---|
| Finnmark | 60 | 5 | 8.3 |
| Troms | 28 | 0 | 0.0 |
| Møre | 67 | 4 | 6.0 |
| Trøndelag | 64 | 19 | 29.7 |
| Rogaland | 20 | 11 | 55.0 |
| Vest-Agder | 60 | 3 | 5.0 |

As the table shows, certain lumpfish populations, in particular those from the Trøndelag and Rogaland counties, have a high CLuCV prevalence.

In view of the disclosure provided herein, it will be appreciated that the present invention also encompasses the following items:
1. A nucleic acid, wherein the sequence of said nucleic acid comprises
   (a) at least one open reading frame (ORF) sequence selected from the group consisting of ORF-1, ORF-2, ORF-3, ORF-4 and ORF-5, or
   (b) a sequence complementary thereto;
      wherein
   ORF-1 is at least 80% identical to the nucleic acid sequence of SEQ ID NO:1,
   ORF-2 is at least 80% identical to the nucleic acid sequence of SEQ ID NO:2,
   ORF-3 is at least 80% identical to the nucleic acid sequence of SEQ ID NO:3,
   ORF-4 is at least 80% identical to the nucleic acid sequence of SEQ ID NO:4, and
   ORF-5 is at least 80% identical to the nucleic acid sequence of SEQ ID NO:5.
2. The nucleic acid of item 1, wherein
   ORF-1 is at least 85% identical to the nucleic acid sequence of SEQ ID NO:1,
   ORF-2 is at least 85% identical to the nucleic acid sequence of SEQ ID NO:2,
   ORF-3 is at least 85% identical to the nucleic acid sequence of SEQ ID NO:3,
   ORF-4 is at least 85% identical to the nucleic acid sequence of SEQ ID NO:4, and
   ORF-5 is at least 85% identical to the nucleic acid sequence of SEQ ID NO:5.
3. The nucleic acid of item 1 or 2, wherein
   ORF-1 is at least 90% identical to the nucleic acid sequence of SEQ ID NO:1,
   ORF-2 is at least 90% identical to the nucleic acid sequence of SEQ ID NO:2,
   ORF-3 is at least 90% identical to the nucleic acid sequence of SEQ ID NO:3,
   ORF-4 is at least 90% identical to the nucleic acid sequence of SEQ ID NO:4, and
   ORF-5 is at least 90% identical to the nucleic acid sequence of SEQ ID NO:5.
4. The nucleic acid of any one of items 1-3, wherein
   ORF-1 is at least 95% identical to the nucleic acid sequence of SEQ ID NO:1,
   ORF-2 is at least 95% identical to the nucleic acid sequence of SEQ ID NO:2,
   ORF-3 is at least 95% identical to the nucleic acid sequence of SEQ ID NO:3,
   ORF-4 is at least 95% identical to the nucleic acid sequence of SEQ ID NO:4, and
   ORF-5 is at least 95% identical to the nucleic acid sequence of SEQ ID NO:5.
5. The nucleic acid of any one of items 1-4, wherein
   ORF-1 is at least 98% identical to the nucleic acid sequence of SEQ ID NO:1,
   ORF-2 is at least 98% identical to the nucleic acid sequence of SEQ ID NO:2,
   ORF-3 is at least 98% identical to the nucleic acid sequence of SEQ ID NO:3,
   ORF-4 is at least 98% identical to the nucleic acid sequence of SEQ ID NO:4, and
   ORF-5 is at least 98% identical to the nucleic acid sequence of SEQ ID NO:5.
6. The nucleic acid of any one of items 1-5, wherein
   ORF-1 is at least 99% identical to the nucleic acid sequence of SEQ ID NO:1,
   ORF-2 is at least 99% identical to the nucleic acid sequence of SEQ ID NO:2,
   ORF-3 is at least 99% identical to the nucleic acid sequence of SEQ ID NO:3,
   ORF-4 is at least 99% identical to the nucleic acid sequence of SEQ ID NO:4, and
   ORF-5 is at least 99% identical to the nucleic acid sequence of SEQ ID NO:5.
7. The nucleic acid of any one of items 1-6, wherein
   ORF-1 is 100% identical to the nucleic acid sequence of SEQ ID NO:1,
   ORF-2 is 100% identical to the nucleic acid sequence of SEQ ID NO:2,
   ORF-3 is 100% identical to the nucleic acid sequence of SEQ ID NO:3,
   ORF-4 is 100% identical to the nucleic acid sequence of SEQ ID NO:4, and
   ORF-5 is 100% identical to the nucleic acid sequence of SEQ ID NO:5.
8. A nucleic acid, wherein the sequence of said nucleic acid is at least 80% identical to a corresponding sequence present within SEQ ID NO:6, preferably at least 85% identical, more preferably at least 90% identical, yet more preferably at least 95% identical, yet more preferably at least 98% identical, yet even more preferably at least 99% identical, and particularly preferably 100% identical.
9. A nucleic acid, wherein the sequence of said nucleic acid is at least 80% identical to a corresponding sequence present within a sequence that is complementary to SEQ ID NO:6, preferably at least 85% identical, more preferably at least 90% identical, yet more preferably at least 95% identical, yet more preferably at least 98% identical, yet even more preferably at least 99% identical, and particularly preferably 100% identical.
10. The nucleic acid of items 8 or 9, wherein the sequence of said nucleic acid comprises 200 nucleotides or less.
11. The nucleic acid of any one of items 8 to 10, wherein the sequence of said nucleic acid comprises at least 60 nucleotides.
12. The nucleic acid of item 11, wherein the sequence of said nucleic acid comprises at least 100 nucleotides, preferably at least 150 nucleotides.
13. The nucleic acid of item 8 or 9, wherein the sequence of said nucleic acid comprises at least 200 nucleotides.
14. Use of the nucleic acid of any one of items 1-13
   (a) as a hybridization probe;
   (b) for detecting the virus disclosed herein in a biological sample of a fish, particularly lumpfish;
   (c) in a method for detecting a virus that infects and is capable of killing fish, particularly lumpfish;
   (d) in a method for detecting a virus that infects and is capable of killing fish, particularly lumpfish, according to any of the corresponding methods disclosed herein;
   (e) for preparing a vaccine for protecting fish, particularly lumpsucker fish, against disease caused by Coronavirus, specifically Torovirus, infection; or
   (f) for preparing a vaccine for protecting fish, particularly lumpsucker fish, against disease caused by a virus disclosed herein, such as the virus of items 18-25 below.
15. The nucleic acid of item 1, wherein the sequence of said nucleic acid comprises at least ORF-1, ORF-2, ORF-3 and ORF-4, as defined in any of items 1 to 7, or a sequence that is complementary thereto.
16. The nucleic acid of item 1, wherein the sequence of said nucleic acid is at least 80% identical to the virus genome according to SEQ ID NO:6, preferably at least 85% identical, more preferably at least 90% identical, yet more preferably at least 95% identical, yet more preferably at least 98% identical, yet even more preferably at least 99% identical, and particularly preferably 100% identical.
17. The nucleic acid of item 1, wherein the sequence of said nucleic acid is at least 80% identical a sequence that is complementary to SEQ ID NO:6, preferably at least 85% identical, more preferably at least 90% identical, yet more preferably at least 95% identical, yet more preferably at least 98% identical, yet even more preferably at least 99% identical, and particularly preferably 100% identical.
18. A virus, in particular a virus that infects and is capable of killing lumpsucker fish (such as Cyclopterus lumpus), wherein the virus genome comprises the nucleic acid sequence of the nucleic acid of any one of items 1 to 13 and 15 to 17, wherein said nucleic acid sequence comprised in the virus genome contains the base uracil (U) instead of the base thymine (T).
19. The virus of item 18, wherein the infection of lumpsucker fish by the virus causes the following symptoms in the fish:
   (i) tissue damage in the intestine, and/or
   (ii) diarrhoea.
20. A virus that comprises one or more, preferably two or more, more preferably three or more, more preferably four or more, yet more preferably five of ORFs 1-5, wherein said ORFs 1-5 encode viral polypeptides of SEQ ID NOs 7-11, respectively, or viral polypeptides that are at least 80% identical to SEQ ID NOs 7-11, respectively.
21. The virus of item 20, wherein said ORFs 1-5 encode viral polypeptides of SEQ ID NOs 7-11, respectively, or viral polypeptides that are at least 90% identical to SEQ ID NOs 7-11, respectively.
22. The virus of item 20, wherein said ORFs 1-5 encode viral polypeptides of SEQ ID NOs 7-11, respectively, or viral polypeptides that are at least 95% identical to SEQ ID NOs 7-11, respectively.
23. The virus of any one of items 20-22, wherein said ORFs 1-5 encode viral polypeptides that are conservatively substituted variants of SEQ ID NOs 7-11, respectively.
24. The virus of any one of items 18-23, wherein the virus is an enveloped virus.
25. The virus of any one of items 18 to 24, wherein the virus is a Coronavirus, preferably a Torovirus.
26. An oligonucleotide primer which comprises a sequence of at least 9 consecutive nucleotides, preferably of at least 12 consecutive nucleotides, more preferably of at least 15 consecutive nucleotides, and particularly preferably of at least 18 consecutive nucleotides, wherein said sequence is comprised within the genome of the virus of any one of items 18 to 25, preferably with the proviso that said primer does not comprise SEQ ID NOs 21-23.
27. An oligonucleotide primer which comprises a sequence of at least 9 consecutive nucleotides, preferably of at least 12 consecutive nucleotides, more preferably of at least 15 consecutive nucleotides, and particularly preferably of at least 18 consecutive nucleotides, wherein said sequence is complementary to a nucleic acid sequence comprised within the genome of the virus of any one of items 18 to 25, preferably with the proviso that said primer does not comprise SEQ ID NOs 21-23.
28. An oligonucleotide primer which comprises a sequence of at least 9 consecutive nucleotides, preferably of at least 12 consecutive nucleotides, more preferably of at least 15 consecutive nucleotides, and particularly preferably of at least 18 consecutive nucleotides, wherein said sequence is at least 80% identical to a sequence comprised within SEQ ID NO:6, preferably with the proviso that said primer does not comprise SEQ ID NOs 21-23.
29. An oligonucleotide primer which comprises a sequence of at least 9 consecutive nucleotides, preferably of at least 12 consecutive nucleotides, more preferably of at least 15 consecutive nucleotides, and particularly preferably of at least 18 consecutive nucleotides, wherein said sequence is at least 80% identical to a sequence comprised within a sequence complementary to SEQ ID NO:6, preferably with the proviso that said primer does not comprise SEQ ID NOs 21-23.
30. The oligonucleotide primer of item 28 or 29, wherein said percentage sequence identity is at least 85%, preferably at least 90%, more preferably at least 95%, yet more preferably at least 98%, yet even more preferably at least 99%, and particularly preferably 100%.
31. The oligonucleotide primer of any one of items 26 to 30, wherein said oligonucleotide primer is 9 to 60 nucleotides in length, preferably 12 to 40 nucleotides in length, more preferably 15 to 30 nucleotides in length, particularly preferably 18 to 25 nucleotides in length.
32. The oligonucleotide primer of any one of items 26 to 28, wherein said sequence comprised therein represents, or is complementary to, a portion of a reference nucleic acid sequence selected from the group consisting of SEQ ID NO:1, SEQ ID NO:2, SEQ ID NO:3, SEQ ID NO:4, SEQ ID NO:5 or SEQ ID NO:6.
33. An oligonucleotide primer which comprises at least 9 consecutive nucleotides, preferably at least 12 consecutive nucleotides, more preferably at least 15 consecutive nucleotides, and particularly preferably at least 18 consecutive nucleotides, of a sequence which is at least 80% identical to a sequence which is, or which is complementary to, a sequence selected from the group consisting of SEQ ID NO:12 to SEQ ID NO:80, preferably with the proviso that said primer does not comprise SEQ ID NOs 21-23.
34. Use of at least one oligonucleotide primer in a method of detecting the virus of any one of items 18 to 25, wherein the at least one primer comprises a sequence of at least 9 consecutive nucleotides, preferably of at least 12 consecutive nucleotides, more preferably of at least 15 consecutive nucleotides, and particularly preferably of at least 18 consecutive nucleotides, and wherein said sequence is complementary to a nucleic acid sequence which is comprised within the genome of said virus, preferably with the proviso that said primer does not comprise SEQ ID NOs 21-23.
35. Use of at least one nucleic acid of any one of items 1 to 13 or 15 to 17, or of at least one oligonucleotide primer of any one of items 26 to 34 in a method of detecting a fish virus, optionally wherein the virus infects and is capable of killing lumpsucker fish.
36. The use of item 35, wherein said nucleic acid, or said oligonucleotide primer is used in a method of detecting the virus of any one of items 18 to 24.
37. A method for detecting a virus that infects and is capable of killing fish, in particular lumpsucker fish, comprising the steps of:
   (a) contacting a nucleic acid extracted from a biological sample of a fish with at least one oligonucleotide primer to form a mixture, wherein the at least one oligonucleotide primer is complementary to a nucleic acid sequence which is comprised within the genome of the virus of any one of items 18 to 24,
   (b) determining whether upon subjecting the mixture of a) to amplification an amplification product is present, wherein the presence of amplification product indicates the presence of RNA associated with the virus, and hence the presence of the virus in the biological sample.
38. The method of item 37, wherein the oligonucleotide primer is an oligonucleotide primer of any one of items 26 to 34.
39. The method of items 37 or 38, wherein the nucleic acid in step (a) of the method, for example RNA, is extracted from biological samples by using solid-phase extraction, e.g., on-column purification using a solid phase of silica gel membrane.
40. The method of any one of items 37 to 38, wherein the nucleic acid in step (a) of the method, for example RNA, is extracted from biological samples by using phenol/chloroform extraction.
41. A method for detecting a virus that infects and is capable of killing fish, in particular lumpsucker fish, comprising the steps of:
   (a) sequencing a nucleic acid extracted from a biological sample of a fish, and
   (b) comparing the resulting nucleic acid sequence with a nucleic acid sequence which is, or which is complementary to, a reference sequence selected from the group consisting of SEQ ID NO:1, SEQ ID NO:2, SEQ ID NO:3, SEQ ID NO:4, SEQ ID NO:5 and SEQ ID NO:6, wherein an at least 80% sequence identity between the two sequences indicates the presence of the virus in the biological sample.
42. The method of item 41, wherein the percentage sequence identity between the two sequences that indicates the presence of the virus in the biological sample is at least 85%, preferably at least 90%, more preferably at least 95%, yet more preferably at least 98%, yet even more preferably at least 99%, and particularly preferably 100%.
43. A viral polypeptide comprising an amino acid sequence that is at least 80% identical to any one of SEQ ID NOs 7-11.
44. The viral polypeptide according to item 43, comprising an amino acid sequence that is at least 90% identical to any one of SEQ ID NOs 7-11.
45. The viral polypeptide according to item 43 or 44, comprising an amino acid sequence that is at least 95% identical to any one of SEQ ID NOs 7-11.
46. The viral polypeptide according to any one of items 43-45, comprising an amino acid sequence comprising any one of SEQ ID NO: 7-11 or a conservatively substituted variant thereof.
47. The viral polypeptide according to any one of items 43-45, comprising an amino acid sequence comprising SEQ ID NOs 7-11.
48. An antibody that binds a polypeptide, wherein the polypeptide is encoded by a nucleic acid sequence which is comprised within the genome of the virus of any one of items 18 to 25, or is a viral polypeptide encoded by a nucleic acid of any one of items 1-13 and 15-17.
49. An antibody that binds a polypeptide selected from the group consisting of:
   (i) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, SEQ ID NO:7;
   (ii) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, SEQ ID NO:8;
   (iii) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, SEQ ID NO:9;
   (iv) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, SEQ ID NO:10; and
   (v) a polypeptide comprising an amino acid sequence which is at least 80%, preferably at least 85%, more preferably at least 90%, yet more preferably at least 95%, yet even more preferably at least 98%, particularly preferably at least 99%, or even 100% identical to, SEQ ID NO:11.
50. A kit for detecting a virus in a biological sample from fish, wherein the kit comprises a nucleic acid of any one of items 1-13 or 15 to 17, an oligonucleotide primer of any one of items 26 to 34 and/or an antibody of item 48 or item 49.
51. The kit of item 50, wherein the kit is suitable to conduct, or is for use for conducting, a real-time RT-PCR assay.
52. The antibody of item 48 or item 49 for use in treating fish infected with a virus, in particular lumpsucker fish.
53. The antibody of item 452, wherein the virus is the virus of any one of items 18 to 25.
54. Use of the virus of any one of items 18 to 25 for producing a vaccine.
55. A vaccine comprising:
   (i) a nucleic acid sequence which is comprised within the genome of the virus of any one of items 18 to 25;
   (ii) a nucleic acid sequence of any one of items 1 to 13 or 15 to 17;
   (iii) a viral polypeptide encoded by a nucleic acid sequence comprised within the genome of the virus of any one of items 18 to 25;
   (iv) a viral polypeptide encoded by a nucleic acid sequence of any one of items 1 to 13 or 15 to 17;
   (v) a viral polypeptide according to any one of items 43-47, or
   (vi) a virus of any one of items 18 to 25.
56. The vaccine of item 55, wherein the sequence of the nucleic acid is the sequence of the nucleic acid referred to in any one of items 1 to 13 or 17, wherein said nucleic acid sequence contains the base uracil (U) instead of the base thymine (T).
57. An interfering RNA (iRNA) molecule for use in treating fish infected with a virus, particularly lumpsucker fish, wherein the iRNA molecule comprises at least 12 (preferably contiguous) nucleotides of, or complementary to, a nucleic acid sequence comprised within the genome of the virus of any one of items 18 to 25.
58. An interfering RNA (iRNA) molecule for use in treating fish infected with a virus, particularly lumpsucker fish, wherein the iRNA molecule comprises at least 12 (preferably contiguous) nucleotides of, or complimentary to, a nucleic acid sequence which is at least 80%, preferably at least 85%, more preferably at least 90%, even more preferably at least 95%, yet even more preferably 98%, particularly preferably 99%, or even 100% identical to the sequence of the virus genome according to SEQ ID NO:6 (CLuCV).
59. The interfering RNA of item 57 or item 58, wherein said iRNA molecule comprises at least 15 and more preferably at least 18 of said (preferably contiguous) nucleotides.

Those skilled in the art will recognize, or be able to ascertain using no more than routine experimentation and/or common general knowledge, numerous equivalents to the specific aspects, items and embodiments disclosed herein both in the Examples and in the body of the entire patent description. Such equivalents are considered to be within the scope of this invention and are intended to be encompassed by the following claims, or any claims that may be pursued based on the present disclosure.

1. A nucleic acid, wherein the sequence of said nucleic acid comprises at least one open reading frame (ORF) sequence selected from the group consisting of ORF-1, ORF-2, ORF-3, ORF-4 and ORF-5; wherein
ORF-1 is at least 80% identical to the nucleic acid sequence of SEQ ID NO:1,
ORF-2 is at least 80% identical to the nucleic acid sequence of SEQ ID NO:2,
ORF-3 is at least 80% identical to the nucleic acid sequence of SEQ ID NO:3,
ORF-4 is at least 80% identical to the nucleic acid sequence of SEQ ID NO:4, and
ORF-5 is at least 80% identical to the nucleic acid sequence of SEQ ID NO:5.

2. The nucleic acid of claim 1, wherein
(a) the sequence of said nucleic acid comprises at least ORF-1, ORF-2, ORF-3 and ORF-4, as defined in claim 1; and/or
(b) the sequence of said nucleic acid is at least 80% identical to the virus genome according to SEQ ID NO:6.

3. A nucleic acid, wherein
(a) the sequence of said nucleic acid is complementary to any one of SEQ ID NO:1 to SEQ ID NO:5, and/or
(b) the sequence of said nucleic acid is complementary to SEQ ID NO:6.

4. A virus that infects and is capable of killing lumpsucker fish (Cyclopterus lumpus), wherein the virus genome comprises the sequence of the nucleic acid of claim 1, wherein said nucleic acid sequence contains the base uracil (U) instead of the base thymine (T).

5. The virus of claim 4, wherein said virus comprises ORF-1, ORF-2, ORF-3, ORF-4 and ORF-5 as defined in claim 1, wherein said ORF-1, ORF-2, ORF-3, ORF-4 and ORF-5 encode viral polypeptides that are at least 80% identical to SEQ ID NOs 7-11.

6. The virus of claim 5, wherein said ORF-1, ORF-2, ORF-3, ORF-4 and ORF-5 encode viral polypeptides that are at least 95% identical to SEQ ID NOs 7-11, respectively.

7. The virus of claim 5, wherein said ORF-1, ORF-2, ORF-3, ORF-4 and ORF-5 encode viral polypeptides that are conservatively substituted variants of SEQ ID NOs 7-11, respectively, or viral polypeptides comprising the amino acid sequences of SEQ ID NOs 7-11, respectively.

8. The virus of claim 4, wherein the infection of lumpsucker fish by the virus causes the following symptoms in the fish:
(i) tissue damage in the intestine, and/or
(ii) diarrhoea.

9. The virus of claim 4, wherein
(a) the virus is an enveloped virus; and/or
(b) the virus is a Coronavirus, preferably a Torovirus.

10. An oligonucleotide primer which
(a) comprises a sequence of at least 9 consecutive nucleotides, wherein said sequence is complementary to a nucleic acid sequence which is comprised within the genome of the virus of claim 4,
(b) comprises at least 9 consecutive nucleotides of a sequence which is, or which is complementary to, a portion of a reference nucleic acid sequence selected from the group consisting of SEQ ID NO:1, SEQ ID NO:2, SEQ ID NO:3, SEQ ID NO:4, SEQ ID NO:5 or SEQ ID NO:6, or
(b) comprises at least 9 consecutive nucleotides of a sequence which is at least 80% identical to a sequence which is, or which is complementary to, a sequence selected from the group consisting of SEQ ID NO:12 to SEQ ID NO:80;
with the proviso that said oligonucleotide primer does not comprise a sequence selected from the group consisting of SEQ ID NO:21 to SEQ ID NO:23.

11. (canceled)

12. A method for detecting a virus that infects and is capable of killing fish, comprising the steps of:
(a) contacting a nucleic acid extracted from a biological sample of a fish with at least one oligonucleotide primer to form a mixture, wherein the at least one oligonucleotide primer is complementary to a nucleic acid sequence which is comprised within the genome of the virus of claim 4,
(b) determining whether upon subjecting the mixture of a) to amplification an amplification product is present, wherein the presence of amplification product indicates the presence of RNA associated with the virus, and hence the presence of the virus in the biological sample.

13. A method for detecting a virus that infects and is capable of killing fish, comprising the steps of:
(a) sequencing a nucleic acid extracted from a biological sample of a fish, and
(b) comparing the resulting nucleic acid sequence with a nucleic acid sequence which is, or which is complementary to, a reference sequence selected from the group consisting of SEQ ID NO:1, SEQ ID NO:2, SEQ ID NO:3, SEQ ID NO:4, SEQ ID NO:5 and SEQ ID NO:6, wherein an at least 80% sequence identity between the two sequences indicates the presence of the virus in the biological sample.

14. An antibody that binds a polypeptide, wherein the polypeptide is encoded by a nucleic acid sequence which is comprised within the genome of the virus of claim 1,
wherein the polypeptide is selected from the group consisting of:
(i) a polypeptide comprising an amino acid sequence which is at least 80% identical to SEQ ID NO:7;
(ii) a polypeptide comprising an amino acid sequence which is at least 80% identical to SEQ ID NO:8;
(iii) a polypeptide comprising an amino acid sequence which is at least 80% identical to SEQ ID NO:9;
(iv) a polypeptide comprising an amino acid sequence which is at least 80% identical to SEQ ID NO:10; and
(v) a polypeptide comprising an amino acid sequence which is at least 80% identical to SEQ ID NO:11.

15. A kit for detecting a virus in a biological sample from fish, wherein the kit comprises an oligonucleotide primer of claim 10,
optionally wherein the kit is a real-time RT-PCR assay.

16. (canceled)

17. (canceled)

18. A viral polypeptide comprising an amino acid sequence that is at least 80% identical to any one of SEQ ID NOs 7-11.

19. The viral polypeptide according to claim 18, comprising an amino acid sequence that is least 90% identical to any one of SEQ ID NOs 7-11.

20. The viral polypeptide according to claim 18, comprising an amino acid sequence that is least 95% identical to any one of SEQ ID NOs 7-11.

21. The viral polypeptide according to claim 18, comprising an amino acid sequence comprising any one of SEQ ID NO: 7-11 or a conservatively substituted variant thereof.

22. The viral polypeptide according to claim 18, comprising an amino acid sequence comprising any one of SEQ ID NOs 7-11.

23. A vaccine comprising:
(i) a nucleic acid as claimed in claim 1;
(ii) a viral polypeptide according to claim 18; or
(iii) a virus of claim 4.

24. The vaccine of claim 23, wherein the sequence of the nucleic acid is the sequence of the nucleic acid claimed in claim 1, wherein said nucleic acid sequence contains the base uracil (U) instead of the base thymine (T).

25. An interfering RNA (iRNA) molecule for use in treating fish infected with a virus, wherein the iRNA molecule comprises at least 12 consecutive nucleotides of, or complimentary to, a nucleic acid sequence which is at least 80% identical to the sequence of the virus genome according to SEQ ID NO:6 (CLuCV).
